(19) United States
(12) Patent Application Publication
(10) Pub. No.: US 2026/0327301 A1
(43) Pub. Date: Oct. 22, 2026

(54) ANTI-IL-31 ANTIBODY AND USE THEREOF

(71) Applicant: NINGBO BEIAN BIOTECHNOLOGY CO., LTD., Ningbo (CN)

(72) Inventors: Caiying MEI, Ningbo (CN); Haiyang LIANG, Ningbo (CN); Mingliang DENG, Ningbo (CN); Jieting LV, Ningbo (CN); Jiamin JIN, Ningbo (CN); Aike WANG, Ningbo (CN); Wei ZHANG, Ningbo (CN); Wei XU, Ningbo (CN); Lu CHEN, Ningbo (CN); Zhihong DAI, Ningbo (CN); Fang BAO, Ningbo (CN)

(73) Assignee: NINGBO BEIAN BIOTECHNOLOGY CO., LTD., Ningbo (CN)

(21) Appl. No.: 19/688,603

(22) Filed: May 27, 2026

Related U.S. Application Data
(63) Continuation of application No. PCT/CN2024/132355, filed on Nov. 15, 2024.

(30) Foreign Application Priority Data
Nov. 27, 2023 (CN) 202311605878.3

(51) Int. Cl. C07K 16/24; A61P 17/04; G01N 33/68

(52) U.S. Cl. CPC C07K 16/244; A61P 17/04; G01N 33/6869; C07K 2317/24; G01N 2333/54

(57) ABSTRACT

An anti-IL-31 antibody and a use thereof are provided. An antibody or an antigen-binding fragment thereof that specifically binds to canine IL-31 includes heavy chain and light chain variable regions. The heavy chain variable region includes: HCDR1, having an amino acid sequence as shown in any one of SEQ ID NOS: 1-10; HCDR2, having an amino acid sequence as shown in any one of SEQ ID NOS: 11-20; and HCDR3, having an amino acid sequence as shown in any one of SEQ ID NOS: 21-30. The light chain variable region includes: LCDR1, having an amino acid sequence as shown in any one of SEQ ID NOS: 31-40; LCDR2, having an amino acid sequence as shown in any one of SEQ ID NOS: 41-50; and LCDR3, having an amino acid sequence as shown in any one of SEQ ID NOS: 51-60.

### CROSS-REFERENCE OF THE RELATED APPLICATIONS

This application is a continuation application of International Application No. PCT/CN2024/132355, filed on Nov. 15, 2024, which is based upon and claims priority to Chinese Patent Application No. 202311605878.3, filed on Nov. 27, 2023, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBZD077_SequenceListing.xml, created on May 26, 2026, and is 211,319 bytes in size.

### TECHNICAL FIELD

The present disclosure relates to the fields of genetic engineering and immunology, and specifically to an anti-IL-31 antibody and uses thereof.

### BACKGROUND

Canine atopic dermatitis is a genetically predisposed inflammatory and pruritic allergic skin disease in canines. Statistically, approximately 450 million canines worldwide suffer from atopic dermatitis, with the incidence rate as high as 10%-15% (Kinga Gortel et al., Can Vet J, 2018 September; 59 (9): 1013-1016). In the United Kingdom, Labrador Retrievers and Golden Retrievers have almost a 50% risk of developing atopic dermatitis due to their genetic background. Certain breeds, such as Golden Retrievers, Labradors, Pit Bulls, Pugs, Boxers, and German Shepherds, exhibit a significantly higher risk of onset than other breeds, and breed susceptibility varies with geographic location (Natalie Katharina Yvonne Gedon et al., Clin Transl Allergy, 2018 October; 8:41). The typical onset age of atopic dermatitis ranges from 6 months to 3 years. Food, environmental allergens, and genetic factors are the primary causes of atopic dermatitis, which distinguishes it from parasitic allergy. Affected canines mostly present with erythema and pruritus, and secondary symptoms include pain and inflammatory infection. Pruritus and inflammatory infection can lead to spontaneous alopecia, excoriation and secondary infection, manifested as papules, pustules and crusts. Common lesion sites include the axillae, ventral abdomen, distal extremities, inner pinnae, as well as the periorbital, perioral and perianal regions.

Canines with atopic dermatitis have defective skin barriers. Allergens penetrate the defective skin and interact with immune cells, which then release various pruritogenic chemicals, in turn inducing pruritus. Multiple other chemical mediators are simultaneously released during the allergic reaction, causing cutaneous inflammation that further impairs barrier function, which in turn increases allergen penetration and leads to secondary infection and bacterial overgrowth. Among these, interleukin-31 (IL-31) is the major cytokine associated with atopic inflammation; others such as IL-4, IL-13 and TNF-α can also affect lipid synthesis to further weaken the epidermal barrier function (Jackeline Franco et al., Metabolites, 2021 Sep. 30; 11 (10): 670). Serum IL-31 concentration is closely correlated with the severity of skin lesions in canines. When affected canines are exposed to allergens, T helper 2 (Th2) cells in the skin produce the cytokine IL-31. IL-31 binds to the heterodimeric receptor composed of IL-31 receptor A (IL-31RA) and oncostatin M receptor (OSMR), activates the STAT signaling cascade via Janus kinase (JAK) phosphorylation, upregulates target gene expression, and elicits pruritic behavior in canines (Eniko Sonkoly et al., J Allergy Clin Immunol, 2006 February; 117 (2): 411-417).

In addition, IL-31 can stimulate the production of inflammatory mediators by promoting epithelial cell responses, as demonstrated in transgenic mice overexpressing IL-31. IL-31 receptor mRNA is most highly expressed in dorsal root ganglia. IL-31 orchestrates interactions among immune cells (T cells, mast cells, eosinophils) and epithelial cells (Jan J. Cornelissen et al., Nat Rev Clin Oncol, 2012 October; 9 (10): 579-590). IL-31 levels are elevated in pruritic allergic skin conditions and can induce scratching behavior in multiple species (e.g., mice, monkeys and canines). Studies in laboratory Beagles have shown that IL-31 administration via several routes (intradermal, subcutaneous, and intravenous) can induce intense pruritic behavior within minutes to hours, indicating that the IL-31 injection model can be used to evaluate the antipruritic efficacy of therapeutic agents for canines (Matthew Krautmann et al., Vet Immunol Immunopathol, 2023 April; 258:110574).

Therefore, canine atopic dermatitis is not a single disease, but a complex syndrome consisting of multiple phenotypes. Monitoring and therapeutic progress for atopic dermatitis may include the use of the Canine Atopic Dermatitis Extent and Severity Index (CADESI) to assess lesion severity, pruritus and skin biophysical parameters (J R Calesso et al., Pol J Vet Sci, 2023 June; 26 (2): 231-238). Current pharmacologic treatments include glucocorticoids, cyclosporine, essential fatty acids and antihistamines. For long-term treatment, cyclosporine may have a slow onset of action and increase costs for pet owners. Although glucocorticoids effectively control allergic pruritus and inflammation in the short term, they may cause secondary adverse reactions with a high incidence, making them unsuitable for long-term treatment (Clarissa P Souza et al., Vet Dermatol, 2018 December; 29 (6): 489). In addition, desensitization therapy can be used for treatment. Allergens are administered by injection once or twice a month for 1 to 2 years, which can reduce allergic reactions and induce immune tolerance to allergens. As a non-pharmacological treatment, it is harmless to the body and has a low probability of enabling canines to achieve lifelong allergen-specific tolerance. However, desensitization therapy must be performed exclusively by veterinary dermatology specialists.

Currently, the latest treatments for canine atopic dermatitis include selective Janus kinase 1 (JAK1) inhibitors and caninized anti-interleukin-31 monoclonal antibodies. Anti-IL-31 antibodies have been proven to reduce or abolish IL-31-induced pruritus in NC/Nga mice (a mouse model of human atopic dermatitis). The anti-canine IL-31 antibody Cytopoint® has been demonstrated to effectively treat allergic dermatitis and atopic dermatitis in 87.8% of canine allergic dermatitis cases (Gober M, et al., Front Vet Sci, 2022 July; 9:909776). Zoetis' exclusive product Cytopoint achieved retail sales of 800 million to 1 billion US dollars three years after launch. The currently marketed domestic formulation is 20 mg (1 mL)/vial, priced at approximately 600 RMB. Its current monopoly in the domestic market results in high treatment costs for pets. The dosage of Cytopoint is 2 mg/kg; for canines weighing more than 30 kg, more than 3 vials of 20 mg (1 mL) are required for subcutaneous administration, which is inconvenient for clinical practice.

Given the challenges posed by costly diagnostic workups, frequent clinical flares and the requirement for lifelong management of canine atopic dermatitis to owners, pets, and veterinarians, there is an urgent need to develop a novel, more effective, safer and lower-cost anti-IL-31 antibody for canines to improve the quality of life of companion canines.

### SUMMARY

To address the problems existing in the prior art, the purpose of the present disclosure is to provide an anti-IL-31 antibody or an antigen-binding fragment thereof, a preparation method thereof and a medical use thereof.

To achieve the above objective, the present disclosure adopts the following specific schemes:

In one aspect, the present disclosure provides an antibody that specifically binds canine IL-31 or an antigen-binding fragment thereof, including a heavy chain variable region and a light chain variable region, wherein:
the heavy chain variable region includes: HCDR1 having the amino acid sequence set forth in any one of SEQ ID NOS: 3, 1-2, 4-10, or any variant thereof; HCDR2 having the amino acid sequence set forth in any one of SEQ ID NOS: 13, 11-12, 14-20, or any variant thereof; and HCDR3 having the amino acid sequence set forth in any one of SEQ ID NOS: 23, 21-22, 24-30, or any variant thereof;
the light chain variable region includes: LCDR1 having the amino acid sequence set forth in any one of SEQ ID NOS: 33, 31-32, 34-40, or any variant thereof; LCDR2 having the amino acid sequence set forth in any one of SEQ ID NOS: 43, 41-42, 44-50, or any variant thereof; and LCDR3 having the amino acid sequence set forth in any one of SEQ ID NOS: 53, 51-52, 54-60, or any variant thereof.

In another aspect, the present disclosure provides a biological material selected from any one of the following:
(1) a polynucleotide encoding the aforementioned antibody or antigen-binding fragment thereof; or
(2) an expression vector including the polynucleotide of (1); or
(3) a host cell including the polynucleotide of (1) or the expression vector of (2).

In another aspect, the present disclosure provides a method for producing an antibody that specifically binds canine IL-31 or an antigen-binding fragment thereof, including the steps of: culturing the aforementioned host cell, isolating the antibody from the culture, and purifying the antibody.

In another aspect, the present disclosure provides a pharmaceutical composition including the aforementioned antibody or antigen-binding fragment thereof, as well as a pharmaceutically acceptable excipient, diluent or carrier.

In another aspect, the present disclosure provides use of the aforementioned antibody or antigen-binding fragment thereof, the aforementioned biological material, the aforementioned pharmaceutical composition, and the aforementioned kit in the manufacture of a medicament for treating pruritus or allergy in a canine.

In another aspect, the present disclosure provides use of the aforementioned antibody or antigen-binding fragment thereof, the aforementioned biological material, the aforementioned pharmaceutical composition, and the aforementioned kit in the manufacture of a medicament for reducing, inhibiting or neutralizing IL-31 activity in a canine.

Compared with the prior art, the present disclosure achieves at least the following beneficial effects:

The anti-canine IL-31 antibody or antigen-binding fragment thereof provided by the present disclosure can specifically bind to canine IL-31 and exhibits affinity comparable to or better than that of existing commercial products. It has been verified that the antibody or antigen-binding fragment thereof shows favorable biological activity both in cells and in animals, and can effectively alleviate clinical symptoms associated with atopic dermatitis in model animals in a canine pruritus model. Meanwhile, the engineered caninized antibody has a low risk of immunogenicity.

### BRIEF DESCRIPTION OF THE DRAWINGS

FIG. 1 shows the result of SDS-PAGE identification of canine IL-31.

FIG. 2 shows the result of competitive ELISA of B cell culture supernatant and commercially available caninized anti-canine IL-31 monoclonal antibody (positive control).

FIG. 3 shows the result of SDS-PAGE of rabbit-derived antibodies. Lanes 1 to 10 are 38E2-1 (non-reducing), 47C1-1 (non-reducing), 35C9-1 (non-reducing), 19D1-1 (non-reducing), 5H4-2 (non-reducing), 38E2-1 (reducing), 47C1-1 (reducing), 35C9-1 (reducing), 19D1-1 (reducing), 5H4-2 (reducing) group, respectively. M is the marker group.

FIG. 4 shows the result of WB detection of rabbit-derived antibody candidates. Lanes 1 to 5 are 38E2-1 (non-reducing), 47C1-1 (non-reducing), 35C9-1 (non-reducing), 19D1-1 (non-reducing), 5H4-2 (non-reducing) group, respectively.

FIG. 5 shows the profile of BLI affinity assay of rabbit-derived antibodies.

FIG. 6 shows the scores of canine pruritus models established by IL-31 at different doses.

FIG. 7 shows the pruritus scores at 8 days post-administration of commercially available caninized anti-canine IL-31 monoclonal antibody, which indirectly verifies the successful establishment of the canine pruritus model. Meanwhile, commercially available caninized anti-canine IL-31 monoclonal antibody can inhibit IL-31-mediated pruritus in model canines.

FIG. 8 shows the pruritus scores of canine pruritus models after treatment with rabbit-derived antibodies administration.

FIG. 9 shows the construction strategy of caninized antibodies.

FIG. 10 shows the result of SDS-PAGE of chimeric antibodies. Lanes 1 to 6 are 38E2-1 (non-reducing), 47C1-1 (non-reducing), 35C9-1 (non-reducing), 38E2-1 (reducing), 47C1-1 (reducing), 35C9-1 (reducing) group, respectively.

FIG. 11 shows the result of WB detection of chimeric antibodies. Lanes 1 to 3 are 38E2-1 (non-reducing), 47C1-1 (non-reducing), 35C9-1 (non-reducing) group, respectively.

FIG. 12 shows the result of ELISA of chimeric antibodies. “weighting fixed” means fixed weighting.

FIG. 13 shows the result of cytobiological activity assay of chimeric antibodies.

FIG. 14 shows the pruritus scores of canine pruritus models after treatment with chimeric antibodies administration.

FIGS. 15A-15B show the result of SDS-PAGE of caninized antibodies. In FIG. 15A, lanes 1 to 8 are 38E2-1-H3L3 (non-reducing), 38E2-1-H3L2 (non-reducing), 47C1-1-H2L2 (non-reducing), 47C1-1-H2L3 (non-reducing), 38E2-1-H3L3 (reducing), 38E2-1-H3L2 (reducing), 47C1-1-H2L2 (reducing), 47C1-1-H2L3 (reducing) group, respectively. In FIG. 15B, lanes 1 to 4 are 35C9-1-HIL1 (non-reducing), 35C9-1-H1L2 (non-reducing), 35C9-1-H1L1 (reducing), 35C9-1-H1L2 (reducing) group, respectively.

FIGS. 16A-16B show the result of WB detection of caninized antibodies. In FIG. 16A, lanes 1 to 4 are 38E2-1-H3L3 (non-reducing), 38E2-1-H3L2 (non-reducing), 47C1-1-H2L2 (non-reducing), 47C1-1-H2L3 (non-reducing) group, respectively. In FIG. 16B, lanes 1 to 2 are 35C9-1-H1L1 (non-reducing), 35C9-1-H1L2 (non-reducing) group, respectively.

FIG. 17 shows the result of ELISA of caninized antibodies. “weighting fixed” means fixed weighting.

FIG. 18 shows the result of cytobiological activity assay of caninized antibodies.

FIG. 19 shows the pruritus scores of canine pruritus models after treatment with caninized antibodies administration.

FIG. 20 shows the result of drug efficacy verification of caninized antibody 38E2-1-H3L3 at different concentrations within 28 days after administration.

FIG. 21 shows the blood drug concentration determination results of repeated administration of 9 mg/kg caninized antibody 38E2-1-H3L3.

### DETAILED DESCRIPTION OF THE EMBODIMENTS

To illustrate the embodiments or technical solutions of the present disclosure more clearly, the terms and implementations used in the embodiments or technical descriptions are briefly introduced below. Obviously, the implementations described below are merely some of the embodiments of the present disclosure. For those of ordinary skill in the art, other embodiments can be derived from these drawings.

### I. Terms

For ease of understanding the present disclosure, certain technical and scientific terms are specifically defined below. Unless otherwise clearly defined elsewhere in this document, all other technical and scientific terms used herein shall have the meaning commonly understood by one of ordinary skill in the art to which this disclosure pertains.

As used herein, the articles “a” and “an” refer to one or more (i.e., at least one) of the grammatical object to which the article refers. By way of example, “an element” means one element or more than one element.

As used herein, the term “about” means and includes the stated value as well as a range above and below that value. In certain embodiments, the term “about” may refer to a variation of ±0.1%, ±0.5%, ±1%, ±2%, ±3%, ±4%, ±5%, ±6%, ±7%, ±8%, ±9%, or ±10%. In certain embodiments, where applicable, the term “about” means the stated value±one standard deviation of that value.

The endpoints and any values of the ranges disclosed herein are not limited to those precise ranges or values, and such ranges or values shall be construed to include values approximating those ranges or values. For numerical ranges, one or more new numerical ranges may be obtained by combining the endpoint values of each range, between the endpoint values of each range and individual point values, and between individual point values, and such numerical ranges shall be deemed to be specifically disclosed herein.

As used throughout the specification and claims, the term “consisting essentially of” or variations thereof means including all recited components or groups of components, and optionally including additional components of similar or different characteristics relative to the recited components, provided that such additional components do not materially alter the basic or novel characteristics of the specified dosage regimen, method, or composition.

“Optional” or “optionally” means that the subsequently described event or circumstance may, but need not, occur, and that the description includes instances where the event or circumstance occurs and instances where it does not.

The term “antibody” refers to any form of antibody that exhibits the desired biological or binding activity. Accordingly, the term is used in its broadest sense and specifically includes, but is not limited to, monoclonal antibodies (including full-length monoclonal antibodies), polyclonal antibodies, humanized antibodies, fully human antibodies, and chimeric antibodies.

Generally, the basic antibody structural unit includes a tetramer. Each tetramer consists of two pairs of identical polypeptide chains, with each pair having one light chain (approximately 25 kDa) and one heavy chain (approximately 50-70 kDa). The amino-terminal portion of each chain includes a variable region of about 100 to 110 or more amino acids that is primarily responsible for antigen recognition. The carboxyl-terminal portion of the heavy chain may define a constant region that is mainly responsible for effector functions. In humans, light chains are generally classified as kappa light chains and lambda light chains. In addition, human heavy chains are commonly divided into mu, delta, gamma, alpha or epsilon, which define the antibody isotypes as IgM, IgD, IgG, IgA and IgE, respectively. In both light and heavy chains, the variable and constant regions are joined by a J region of about 12 or more amino acids, and the heavy chain also contains a D region of over about 10 amino acids. See generally Fundamental Immunology Ch. 7 (Paul, W., ed., 2nd ed. Raven Press, N.Y. (1989)).

The variable regions of each light/heavy chain pair form the antibody-binding site. Thus, a complete antibody generally has two binding sites. Except for bifunctional or bispecific antibodies, these two binding sites are generally identical.

Generally, the variable regions of both heavy and light chains each contain three hypervariable regions, also known as complementarity-determining regions, which are situated within relatively conserved framework regions. The CDR is usually aligned by the framework regions to enable the binding of a specific epitope. Generally, from the N-terminus to the C-terminus, both the light and heavy chain variable domains include FR1, CDR1, FR2, CDR2, FR3, CDR3 and FR4. The assignment of amino acids to each domain generally conforms to the definitions set forth in: Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J. Mol. Biol. 196:901-917; or Chothia, et al., (1989) Nature 342:878-883.

As used herein, the term “antibody fragment” or “antigen-binding fragment” refers to an antigen-binding fragment of an antibody, i.e., a fragment of an antibody that retains the ability to specifically bind to the antigen of the full-length antibody, such as a fragment retaining one or more CDR regions. Examples of antibody-binding fragments include, but are not limited to, Fab, Fab′, F(ab′)₂ and Fv fragments.

As used herein, the term “CDR” refers to the complementarity-determining regions within immunoglobulin variable regions, which are generally defined using the Kabat numbering system.

As used herein, an antibody that “specifically binds” a specific target protein is an antibody that exhibits preferential binding to the target relative to other proteins, though such specificity does not require absolute binding specificity. An antibody is deemed to be “specific” for its intended target if its binding enables the determination of the presence of the target protein in a sample without yielding undesirable results such as false positives. An antibody or binding fragment thereof for use in the present disclosure will bind to a target protein with an affinity that is at least two-fold higher, preferably at least ten fold higher, more preferably at least 20 fold higher, and most preferably at least 100 fold higher than its affinity for non-target proteins. As used herein, if an antibody binds to a polypeptide including a given amino acid sequence, such as the amino acid sequence of a mature human PD-1 or human PD-L1 molecule, but does not bind to a protein lacking the sequence, the antibody is said to specifically bind to the polypeptide including the sequence.

As used herein, the term “chimeric antibody” refers to an antibody in which a portion of the heavy chain and/or light chain is identical or homologous to the corresponding sequence in an antibody derived from a specific species (e.g., human) or belonging to a specific antibody class or subclass, while the remaining portion of the chain is identical or homologous to the corresponding sequence in an antibody derived from another species (e.g., mouse) or belonging to another antibody class or subclass, as well as fragments of such antibodies, provided that they exhibit the desired biological activity.

In the present disclosure, the chimeric antibodies are prepared by splicing the heavy chain variable regions and light chain variable regions of the rabbit-derived antibody molecules 38E2-1, 47C1-1 and 35C9-1 to the heavy chain constant region and light chain constant region of canine IgGB, respectively. Among them, the accession number of the amino acid sequence of canine IgGB heavy chain is AAL35302.1 (GenBank database), and the accession number of its DNA sequence is AF354265.1; the accession number of the amino acid sequence of canine antibody K light chain is ABY 57289.1, and the accession number of its DNA sequence is EU305402.1.

Similarly, the specific preparation process of the caninized antibody is as follows: a caninized template with the highest degree of matching to the FV region of the rabbit-derived antibody is retrieved from the IMGT website, the CDR regions in the caninized template are replaced with the CDR regions of the rabbit-derived antibody, the sequences are analyzed by bioinformatics software, and back mutations are performed on specific amino acids to obtain a caninized antibody with affinity and activity comparable to those of the rabbit-derived antibody. As used herein, the term “conservatively modified variant” or “conservative substitution” refers to the substitution of an amino acid in a protein with another amino acid having similar characteristics (e.g., charge, side chain size, hydrophobicity/hydrophilicity, backbone conformation and rigidity, etc.), such that such alterations can be made frequently without altering the biological activity or other desired properties (e.g., antigen affinity and/or specificity) of the protein. Those skilled in the art recognize that, in general, a single amino acid substitution in a non-essential region of a polypeptide does not significantly alter biological activity. In addition, the substitution of amino acids with similar structural or functional properties is unlikely to disrupt biological activity. Exemplary conservative substitutions are listed in Table 1.

TABLE 1
Exemplary Conservative Amino Acid Substitutions

| Original Residue | Conservative Substitution |
|---|---|
| Ala(A) | Gly; Ser |
| Arg(R) | Lys; His |
| Asn(N) | Gln; His |
| Asp(D) | Glu; Asn |
| Cys(C) | Ser; Ala |
| Gln(Q) | Asn |
| Glu(E) | Asp; Gln |
| Gly(G) | Ala |
| His(H) | Asn; Gln |
| Ile(I) | Leu; Val |
| Leu(L) | Ile; Val |
| Lys(K) | Arg; His |
| Met(M) | Leu; Ile; Tyr |
| Phe(F) | Tyr; Met; Leu |
| Pro(P) | Ala |
| Ser(S) | Thr |
| Thr(T) | Ser |
| Trp(W) | Tyr; Phe |
| Tyr(Y) | Trp; Phe |
| Val(V) | Ile; Leu |

As used herein, the terms “cell”, “cell line” and “cell culture” are used interchangeably, and all such terms include their progeny. Thus, the terms “transformant” and “transformed cell” include the primary subject cell and cultures derived therefrom, regardless of the number of passages. It should also be understood that all progeny may not be precisely identical in DNA content, due to deliberate or inadvertent mutation. Mutant progeny that retain the same function or biological activity as screened for in the originally transformed cell are included. Where distinct designations are intended, it will be clear from the context. In the context of expressing a heterologous nucleic acid sequence, a “host cell” refers to a prokaryotic or eukaryotic cell (e.g., bacterial cell, yeast cell, mammalian cell and insect cell), whether in vitro or in vivo. For example, a host cell may be situated within a transgenic animal. A host cell can serve as a recipient for a vector and may include any transformable organism capable of replicating a vector and/or expressing a heterologous nucleic acid encoded by the vector.

As used herein, the term “pharmaceutical composition” relates to a composition for administration to a mammalian patient (preferably a canine patient). In a preferred embodiment, the pharmaceutical composition includes a composition for parenteral injection or infusion. Such parenteral injection or infusion may take advantage of resorption processes, and said resorption processes are in the form of intradermal, subcutaneous, intramuscular and/or intraperitoneal injection or infusion. Alternatively, such parenteral injection or infusion may bypass the resorption process and be in the form of intracardiac, intraarterial, intravenous, intralumbar and/or intramembranous injection or infusion. In another preferred embodiment, the pharmaceutical composition includes a composition for administration through the skin. One example of administration through the skin is epicutaneous administration, wherein the pharmaceutical composition is administered in the form of a solution, suspension, emulsion, foam, oil ointment, ointment, paste and/or a patch attached to the skin. Alternatively, administration of the pharmaceutical composition may be achieved through one or more mucous membranes. For example, administration may be buccal, lingual or sublingual, i.e., through the mucous membranes of the mouth and/or tongue, and the forms of application may be tablets, lozenges, dragees (i.e., sugar-coated pills) and/or mouth rinse solutions. Alternatively, administration may be enteral, i.e., through the mucous membranes of the stomach and/or intestines, and the forms of application may be tablets, dragees (i.e., sugar-coated pills), capsules, solutions, suspensions and/or emulsions. Alternatively, administration may be rectal, and the forms of application may be suppositories, rectal capsules and/or ointments or oil ointments. Alternatively, administration may be intranasal, and the forms of application may be drops, ointments or oil ointments and/or sprays. Alternatively, administration may be pulmonary, i.e., through the bronchi and/or alveoli, and the forms of application may be aerosols and/or inhalants. Alternatively, administration may be conjunctival, and the forms of application may be eye drops, eye ointments and/or eye wash solutions. Alternatively, administration may be achieved through the mucous membranes of the genitourinary tract, such as intravaginal or intraurethral, and the forms of application may be suppositories, ointments and/or medicated sticks. It should be understood that the above alternative forms of administration are not mutually exclusive, and any number of combinations thereof may be used to form an effective treatment regimen.

The pharmaceutical composition of the present disclosure may further include a pharmaceutically acceptable carrier. Examples of suitable pharmaceutical carriers are well known in the art and include phosphate-buffered saline, water, emulsions (such as oil/water emulsions), various wetting agents, sterile solutions, and the like. Compositions including these carriers can be formulated by known conventional methods. These pharmaceutical compositions can be administered to a subject at a suitable dose. The dosage regimen can be determined by the attending physician and clinical factors. As is well known in the existing medical art, the dosage for any patient depends on many factors, including the patient's size, body surface area, age, the particular compound administered, sex, time and route of administration, general health, and other drugs being administered concurrently. For example, formulations for parenteral administration include sterile aqueous or non-aqueous solutions, suspensions, emulsions and liposomes. Examples of non-aqueous solvents are carriers such as propylene glycol, polyethylene glycol, vegetable oils (such as olive oil), and injectable organic esters (such as ethyl oleate), including sodium chloride solution, Ringer's dextrose, dextrose and sodium chloride, lactated Ringer's, or fixed oils. Carriers suitable for intravenous or intraarterial administration include fluid and nutrient replenishers, electrolyte replenishers (such as those based on Ringer's dextrose), and the like. Preservatives and other additives may also be included, for example, antimicrobial, antioxidant, chelating, inert gas, and the like. In addition, the pharmaceutical composition of the present disclosure may include a protein carrier, such as serum albumin or immunoglobulin, preferably of human origin. It is noted that, in addition to the humanized monoclonal antibody or fragment thereof (as described in the present disclosure), the pharmaceutical composition of the present disclosure may further include other biologically active agents, depending on the intended use of the pharmaceutical composition. The agent may be a drug acting on the gastrointestinal system, a drug acting as a cytostatic agent, a drug preventing hyperuricemia, a drug inhibiting an immune response (such as a corticosteroid), a drug modulating an inflammatory response, a drug acting on the circulatory system, and/or a conventionally known agent such as a cytokine.

As used herein, the terms “subject” or “patient” refer to an animal in need of treatment that can be affected by the molecules of the present disclosure. Animals treatable according to the present disclosure include vertebrates, with mammals such as canine, cats and equines being particularly preferred examples.

As used herein, a “therapeutically effective amount” (or “effective amount”) refers to an amount of an active ingredient (e.g., the agent of the present disclosure) sufficient to produce beneficial or desired results when administered to a subject or patient. An effective amount may be administered in one or more administrations, applications or doses. The therapeutically effective amount of the composition of the present disclosure can be readily determined by one of ordinary skill in the art. In the context of the present disclosure, a “therapeutically effective amount” is an amount that produces an objectively measurable change in one or more parameters associated with the treatment of pruritus or allergic diseases, including clinical improvement of symptoms. Of course, the therapeutically effective amount will vary depending on the particular subject and disease to be treated, the weight and age of the subject, the severity of the disease symptoms, the particular compound selected, the dosing regimen to be followed, the time of administration, the mode of administration, etc., all of which can be readily determined by one of ordinary skill in the art.

As used herein, “therapeutic” encompasses comprehensive treatment of a disease or condition. The “therapeutic” agent of the present disclosure may act in a prophylactic or preventive manner, including procedures designed for animals identifiable as being at risk (pharmacogenetics); or in an ameliorative or substantially therapeutic manner; or may act to slow the rate or extent of progression of at least one symptom of the disease or condition to be treated. The terms “treatment”, “treating” and the like refer to both therapeutic treatment and prophylactic or preventive measures. Animals in need of treatment include those already having the condition as well as those in which the condition is to be prevented. The term “treatment” or “treating” a disease or condition includes prevention or protection against the disease or condition (i.e., preventing clinical symptoms from developing); inhibition of the disease or condition (i.e., arresting or suppressing the development of clinical symptoms); and/or alleviation of the disease or condition (i.e., causing regression of clinical symptoms). As will be understood, it is not always possible to distinguish between “prevention” and “inhibition” of a disease or condition, since the ultimate precipitating event may be unknown or latent. Accordingly, the term “prevention” shall be understood to constitute a category of treatment that encompasses “prevention” and “inhibition”. The term “treatment” thus includes “prevention”.

As used herein, the term “allergic disease” is defined herein as a condition or disease caused by an interaction between the immune system and an external substance. Such foreign substances are called “allergens”. Common allergens include airborne allergens such as pollen, dust, molds, dust mite proteins, saliva injected by insect bites, and the like. Examples of allergic diseases include, but are not limited to, the following: allergic dermatitis, summer eczema, urticaria, equine heaves, inflammatory airway diseases, recurrent airway obstruction, airway hyperresponsiveness, chronic obstructive pulmonary disease, and inflammatory processes caused by autoimmunity, such as irritable bowel syndrome (IBS).

As used herein, the terms “pruritus”, “pruritic disease” are defined herein as a disease or condition characterized by an intense itching sensation that produces the urge to rub or scratch the skin to obtain relief. Examples of pruritic diseases include, but are not limited to, the following: atopic dermatitis, eczema, psoriasis, scleroderma and pruritus.

### II. Detailed Description of Specific Embodiments

In one aspect, the present disclosure provides an antibody that specifically binds canine IL-31 or an antigen-binding fragment thereof, including a heavy chain variable region and a light chain variable region, wherein:
the heavy chain variable region includes: HCDR1 having the amino acid sequence set forth in any one of SEQ ID NOS: 3, 1-2, 4-10, or any variant thereof; HCDR2 having the amino acid sequence set forth in any one of SEQ ID NOS: 13, 11-12, 14-20, or any variant thereof; and HCDR3 having the amino acid sequence set forth in any one of SEQ ID NOS: 23, 21-22, 24-30, or any variant thereof;
the light chain variable region includes: LCDR1 having the amino acid sequence set forth in any one of SEQ ID NOS: 33, 31-32, 34-40, or any variant thereof; LCDR2 having the amino acid sequence set forth in any one of SEQ ID NOS: 43, 41-42, 44-50, or any variant thereof; and LCDR3 having the amino acid sequence set forth in any one of SEQ ID NOS: 53, 51-52, 54-60, or any variant thereof.

In some embodiments, in the antibody or antigen-binding fragment thereof, the heavy chain variable region includes:
(1) HCDR1 as set forth in SEQ ID NO: 3, HCDR2 as set forth in SEQ ID NO: 13, HCDR3 as set forth in SEQ ID NO: 23; or,
(2) HCDR1 as set forth in SEQ ID NO: 1, HCDR2 as set forth in SEQ ID NO: 11, HCDR3 as set forth in SEQ ID NO: 21; or,
(3) HCDR1 as set forth in SEQ ID NO: 2, HCDR2 as set forth in SEQ ID NO: 12, HCDR3 as set forth in SEQ ID NO: 22; or,
(4) HCDR1 as set forth in SEQ ID NO: 4, HCDR2 as set forth in SEQ ID NO: 14, HCDR3 as set forth in SEQ ID NO: 24; or,
(5) HCDR1 as set forth in SEQ ID NO: 5, HCDR2 as set forth in SEQ ID NO: 15, HCDR3 as set forth in SEQ ID NO: 25; or,
(6) HCDR1 as set forth in SEQ ID NO: 6, HCDR2 as set forth in SEQ ID NO: 16, HCDR3 as set forth in SEQ ID NO: 26; or,
(7) HCDR1 as set forth in SEQ ID NO: 7, HCDR2 as set forth in SEQ ID NO: 17, HCDR3 as set forth in SEQ ID NO: 27; or,
(8) HCDR1 as set forth in SEQ ID NO: 8, HCDR2 as set forth in SEQ ID NO: 18, HCDR3 as set forth in SEQ ID NO: 28; or,
(9) HCDR1 as set forth in SEQ ID NO: 9, HCDR2 as set forth in SEQ ID NO: 19, HCDR3 as set forth in SEQ ID NO: 29; or,
(10) HCDR1 as set forth in SEQ ID NO: 10, HCDR2 as set forth in SEQ ID NO: 20, HCDR3 as set forth in SEQ ID NO: 30;
the light chain variable region includes:
(1) LCDR1 as set forth in SEQ ID NO: 33, LCDR2 as set forth in SEQ ID NO: 43, LCDR3 as set forth in SEQ ID NO: 53; or,
(2) LCDR1 as set forth in SEQ ID NO: 31, LCDR2 as set forth in SEQ ID NO: 41, LCDR3 as set forth in SEQ ID NO: 51; or,
(3) LCDR1 as set forth in SEQ ID NO: 32, LCDR2 as set forth in SEQ ID NO: 42, LCDR3 as set forth in SEQ ID NO: 52; or,
(4) LCDR1 as set forth in SEQ ID NO: 34, LCDR2 as set forth in SEQ ID NO: 44, LCDR3 as set forth in SEQ ID NO: 54; or,
(5) LCDR1 as set forth in SEQ ID NO: 35, LCDR2 as set forth in SEQ ID NO: 45, LCDR3 as set forth in SEQ ID NO: 55; or,
(6) LCDR1 as set forth in SEQ ID NO: 36, LCDR2 as set forth in SEQ ID NO: 46, LCDR3 as set forth in SEQ ID NO: 56; or,
(7) LCDR1 as set forth in SEQ ID NO: 37, LCDR2 as set forth in SEQ ID NO: 47, LCDR3 as set forth in SEQ ID NO: 57; or,
(8) LCDR1 as set forth in SEQ ID NO: 38, LCDR2 as set forth in SEQ ID NO: 48, LCDR3 as set forth in SEQ ID NO: 58; or,
(9) LCDR1 as set forth in SEQ ID NO: 39, LCDR2 as set forth in SEQ ID NO: 49, LCDR3 as set forth in SEQ ID NO: 59; or,
(10) LCDR1 as set forth in SEQ ID NO: 40, LCDR2 as set forth in SEQ ID NO: 50, LCDR3 as set forth in SEQ ID NO: 60.

In some embodiments, in the antibody or antigen-binding fragment thereof,
(1) the heavy chain variable region includes HCDR1 as set forth in SEQ ID NO: 3, HCDR2 as set forth in SEQ ID NO: 13, HCDR3 as set forth in SEQ ID NO: 23; the light chain variable region includes LCDR1 as set forth in SEQ ID NO: 33, LCDR2 as set forth in SEQ ID NO: 43, LCDR3 as set forth in SEQ ID NO: 53; or,
(2) the heavy chain variable region includes HCDR1 as set forth in SEQ ID NO: 1, HCDR2 as set forth in SEQ ID NO: 11, HCDR3 as set forth in SEQ ID NO: 21; the light chain variable region includes LCDR1 as set forth in SEQ ID NO: 31, LCDR2 as set forth in SEQ ID NO: 41, LCDR3 as set forth in SEQ ID NO: 51; or,
(3) the heavy chain variable region includes HCDR1 as set forth in SEQ ID NO: 2, HCDR2 as set forth in SEQ ID NO: 12, HCDR3 as set forth in SEQ ID NO: 22; the light chain variable region includes LCDRI as set forth in SEQ ID NO: 32, LCDR2 as set forth in SEQ ID NO: 42, LCDR3 as set forth in SEQ ID NO: 52; or,
(4) the heavy chain variable region includes HCDR1 as set forth in SEQ ID NO: 4, HCDR2 as set forth in SEQ ID NO: 14, HCDR3 as set forth in SEQ ID NO: 24; the light chain variable region includes LCDR1 as set forth in SEQ ID NO: 34, LCDR2 as set forth in SEQ ID NO: 44, LCDR3 as set forth in SEQ ID NO: 54; or,
(5) the heavy chain variable region includes HCDR1 as set forth in SEQ ID NO: 5, HCDR2 as set forth in SEQ ID NO: 15, HCDR3 as set forth in SEQ ID NO: 25; the light chain variable region includes LCDR1 as set forth in SEQ ID NO: 35, LCDR2 as set forth in SEQ ID NO: 45, LCDR3 as set forth in SEQ ID NO: 55; or,
(6) the heavy chain variable region includes HCDR1 as set forth in SEQ ID NO: 6, HCDR2 as set forth in SEQ ID NO: 16, HCDR3 as set forth in SEQ ID NO: 26; the light chain variable region includes LCDR1 as set forth in SEQ ID NO: 36, LCDR2 as set forth in SEQ ID NO: 46, LCDR3 as set forth in SEQ ID NO: 56; or,
(7) the heavy chain variable region includes HCDR1 as set forth in SEQ ID NO: 7, HCDR2 as set forth in SEQ ID NO: 17, HCDR3 as set forth in SEQ ID NO: 27; the light chain variable region includes LCDRI as set forth in SEQ ID NO: 37, LCDR2 as set forth in SEQ ID NO: 47, LCDR3 as set forth in SEQ ID NO: 57; or,
(8) the heavy chain variable region includes HCDR1 as set forth in SEQ ID NO: 8, HCDR2 as set forth in SEQ ID NO: 18, HCDR3 as set forth in SEQ ID NO: 28; the light chain variable region includes LCDR1 as set forth in SEQ ID NO: 38, LCDR2 as set forth in SEQ ID NO: 48, LCDR3 as set forth in SEQ ID NO: 58; or,
(9) the heavy chain variable region includes HCDR1 as set forth in SEQ ID NO: 9, HCDR2 as set forth in SEQ ID NO: 19, HCDR3 as set forth in SEQ ID NO: 29; the light chain variable region includes LCDR1 as set forth in SEQ ID NO: 39, LCDR2 as set forth in SEQ ID NO: 49, LCDR3 as set forth in SEQ ID NO: 59; or,
(10) the heavy chain variable region includes HCDR1 as set forth in SEQ ID NO: 10, HCDR2 as set forth in SEQ ID NO: 20, HCDR3 as set forth in SEQ ID NO: 30; the light chain variable region includes LCDR1 as set forth in SEQ ID NO: 40, LCDR2 as set forth in SEQ ID NO: 50, LCDR3 as set forth in SEQ ID NO: 60.

In some embodiments, in the antibody or antigen-binding fragment thereof, (1) the heavy chain variable region includes HCDR1 as set forth in SEQ ID NO: 1, HCDR2 as set forth in SEQ ID NO: 11, HCDR3 as set forth in SEQ ID NO: 21; the light chain variable region includes LCDR1 as set forth in SEQ ID NO: 31, LCDR2 as set forth in SEQ ID NO: 41, LCDR3 as set forth in SEQ ID NO: 51; or,
(2) the heavy chain variable region includes HCDR1 as set forth in SEQ ID NO: 2, HCDR2 as set forth in SEQ ID NO: 12, HCDR3 as set forth in SEQ ID NO: 22; the light chain variable region includes LCDR1 as set forth in SEQ ID NO: 32, LCDR2 as set forth in SEQ ID NO: 42, LCDR3 as set forth in SEQ ID NO: 52; or,
(3) the heavy chain variable region includes HCDR1 as set forth in SEQ ID NO: 3, HCDR2 as set forth in SEQ ID NO: 13, HCDR3 as set forth in SEQ ID NO: 23; the light chain variable region includes LCDR1 as set forth in SEQ ID NO: 33, LCDR2 as set forth in SEQ ID NO: 43, LCDR3 as set forth in SEQ ID NO: 53.

In some embodiments, the antibody or antigen-binding fragment thereof is selected from any one of a rabbit-derived antibody or antigen-binding fragment thereof, a chimeric antibody or antigen-binding fragment thereof, or a caninized antibody or antigen-binding fragment thereof.

In some embodiments, the antibody or antigen-binding fragment thereof, wherein the antibody that specifically binds canine IL-31 or an antigen-binding fragment thereof includes a heavy chain selected from the sequences set forth as follows, or a heavy chain having at least 80%, 85%, 90%, 95% or 99% identity as compared to the following sequences: SEQ ID NOS: 61-70, 163-165, 175-180; and/or
includes a light chain selected from the sequences set forth as follows, or a light chain having at least 80%, 85%, 90%, 95% or 99% identity as compared to the following sequences: SEQ ID NOS: 71-80, 166-168, 181-186.

In some embodiments, the antibody or antigen-binding fragment thereof, wherein
(1) the heavy chain includes the amino acid sequence set forth in SEQ ID NO: 61 or any variant thereof; the light chain includes the amino acid sequence set forth in SEQ ID NO: 71 or any variant thereof, or,
(2) the heavy chain includes the amino acid sequence set forth in SEQ ID NO: 62 or any variant thereof; the light chain includes the amino acid sequence set forth in SEQ ID NO: 72 or any variant thereof, or,
(3) the heavy chain includes the amino acid sequence set forth in SEQ ID NO: 63 or any variant thereof; the light chain includes the amino acid sequence set forth in SEQ ID NO: 73 or any variant thereof; or,
(4) the heavy chain includes the amino acid sequence set forth in SEQ ID NO: 64 or any variant thereof; the light chain includes the amino acid sequence set forth in SEQ ID NO: 74 or any variant thereof; or,
(5) the heavy chain includes the amino acid sequence set forth in SEQ ID NO: 65 or any variant thereof; the light chain includes the amino acid sequence set forth in SEQ ID NO: 75 or any variant thereof; or,
(6) the heavy chain includes the amino acid sequence set forth in SEQ ID NO: 66 or any variant thereof; the light chain includes the amino acid sequence set forth in SEQ ID NO: 76 or any variant thereof, or,
(7) the heavy chain includes the amino acid sequence set forth in SEQ ID NO: 67 or any variant thereof; the light chain includes the amino acid sequence set forth in SEQ ID NO: 77 or any variant thereof; or,
(8) the heavy chain includes the amino acid sequence set forth in SEQ ID NO: 68 or any variant thereof; the light chain includes the amino acid sequence set forth in SEQ ID NO: 78 or any variant thereof; or,
(9) the heavy chain includes the amino acid sequence set forth in SEQ ID NO: 69 or any variant thereof; the light chain includes the amino acid sequence set forth in SEQ ID NO: 79 or any variant thereof; or,
(10) the heavy chain includes the amino acid sequence set forth in SEQ ID NO: 70 or any variant thereof; the light chain includes the amino acid sequence set forth in SEQ ID NO: 80 or any variant thereof; or,
(11) the heavy chain includes the amino acid sequence set forth in SEQ ID NO: 163 or any variant thereof; the light chain includes the amino acid sequence set forth in SEQ ID NO: 166 or any variant thereof, or,
(12) the heavy chain includes the amino acid sequence set forth in SEQ ID NO: 164 or any variant thereof; the light chain includes the amino acid sequence set forth in SEQ ID NO: 167 or any variant thereof; or,
(13) the heavy chain includes the amino acid sequence set forth in SEQ ID NO: 165 or any variant thereof; the light chain includes the amino acid sequence set forth in SEQ ID NO: 168 or any variant thereof, or,
(14) the heavy chain includes the amino acid sequence set forth in SEQ ID NO: 175 or any variant thereof; the light chain includes the amino acid sequence set forth in SEQ ID NO: 181 or any variant thereof; or,
(15) the heavy chain includes the amino acid sequence set forth in SEQ ID NO: 176 or any variant thereof; the light chain includes the amino acid sequence set forth in SEQ ID NO: 182 or any variant thereof, or,
(16) the heavy chain includes the amino acid sequence set forth in SEQ ID NO: 177 or any variant thereof; the light chain includes the amino acid sequence set forth in SEQ ID NO: 183 or any variant thereof; or,
(17) the heavy chain includes the amino acid sequence set forth in SEQ ID NO: 178 or any variant thereof; the light chain includes the amino acid sequence set forth in SEQ ID NO: 184 or any variant thereof; or,
(18) the heavy chain includes the amino acid sequence set forth in SEQ ID NO: 179 or any variant thereof; the light chain includes the amino acid sequence set forth in SEQ ID NO: 185 or any variant thereof; or,
(19) the heavy chain includes the amino acid sequence set forth in SEQ ID NO: 180 or any variant thereof; the light chain includes the amino acid sequence set forth in SEQ ID NO: 186 or any variant thereof.

In another aspect, the present disclosure provides a biological material selected from any one of the following:
(1) a polynucleotide encoding the aforementioned antibody or antigen-binding fragment thereof; or
(2) an expression vector including the polynucleotide of (1); or
(3) a host cell including the polynucleotide of (1) or the expression vector of (2).

In another aspect, the present disclosure provides a method for producing an antibody that specifically binds canine IL-31 or an antigen-binding fragment thereof, including the steps of: culturing the aforementioned host cell, isolating the antibody from the culture, and purifying the antibody.

In another aspect, the present disclosure provides a pharmaceutical composition including the aforementioned antibody or antigen-binding fragment thereof, as well as a pharmaceutically acceptable excipient, diluent or carrier.

In another aspect, the present disclosure provides use of the aforementioned antibody or antigen-binding fragment thereof, the aforementioned biological material, the aforementioned pharmaceutical composition, and the aforementioned kit in the manufacture of a medicament for treating pruritus or allergy in a canine.

In some embodiments, the use includes alleviating, inhibiting or neutralizing pruritus or allergy in a canine. In some embodiments, the pruritus is atopic dermatitis, eczema, psoriasis or scleroderma. In some preferred embodiments, the pruritus is atopic dermatitis. In some embodiments, the use includes alleviating the clinical manifestations of atopic dermatitis. In some embodiments, the clinical manifestation of atopic dermatitis is pruritus.

In another aspect, the present disclosure provides use of the aforementioned antibody or antigen-binding fragment thereof, the aforementioned biological material, the aforementioned pharmaceutical composition, and the aforementioned kit in the manufacture of a medicament for reducing, inhibiting or neutralizing IL-31 activity in a canine. In some embodiments, the pruritus is atopic dermatitis, eczema, psoriasis or scleroderma. In some preferred embodiments, the pruritus is atopic dermatitis. In some embodiments, the use includes alleviating the clinical manifestations of atopic dermatitis. In some embodiments, the clinical manifestation of atopic dermatitis is pruritus.

In another aspect, the present disclosure provides a method for treating pruritus or allergy in a canine, including administering a therapeutically effective amount of the aforementioned antibody or antigen-binding fragment thereof or the aforementioned pharmaceutical composition to alleviate, inhibit or neutralize IL-31 activity in a canine. In some embodiments, the pruritus is atopic dermatitis, eczema, psoriasis or scleroderma. In some preferred embodiments, the pruritus is atopic dermatitis. In some embodiments, the method includes alleviating the clinical manifestations of atopic dermatitis. In some embodiments, the clinical manifestation of atopic dermatitis is pruritus.

### EXAMPLES

A further understanding of the present disclosure may be obtained by reference to certain specific examples provided herein, which are intended merely to illustrate the present disclosure and are not intended to impose any limitation on the scope of the present disclosure. Obviously, various modifications and variations can be made to the present disclosure without departing from its essence, and accordingly, such modifications and variations are also within the scope of protection claimed in the present application.

### Example 1: Preparation of Rabbit Anti-Canine IL-31 Monoclonal Antibody

### 1. Preparation and Identification of Immunogen

The nucleotide sequence (Gene ID: 100302725, SEQ ID NO: 200) and amino acid sequence (SEQ ID NO: 199) of canine IL-31 were retrieved from the NCBI database. Recombinant canine IL-31 was generated in CHO cells using a stable expression system to produce canine IL-31 protein, which was subsequently used as the immunogen. The recombinantly expressed canine IL-31 protein was subjected to purity and molecular weight analysis by SDS-PAGE, and its activity was identified by ELISA.

### 1.1. SDS-PAGE Assay

The specific experimental procedure was as follows:
(1) Gel plate assembly: The comb was pulled out, the paired gel plates were placed into the mold, and the entire assembly was then placed into the electrophoresis tank. The bottom sealing film of the reducing gel plate was torn off.
(2) Sample treatment: Non-reduced samples: An appropriate amount of sample was taken and mixed with non-reduced 5× loading buffer at a volume ratio of 4:1, followed by boiling in a water bath for 5 min. Reduced samples: An appropriate amount of sample was taken and mixed with reduced 5× loading buffer at a volume ratio of 4:1, followed by boiling in a water bath for 5 min.
(3) Sample loading: The inner and outer tanks of the electrophoresis tank were filled with electrophoresis buffer, and the samples and protein Marker were loaded. The electrophoresis tank was covered, ensuring the red terminal was aligned with the positive electrode and the black terminal with the negative electrode.
(4) Electrophoresis: The power supply was turned on to start electrophoresis. For gradient gels, the initial voltage was set to 90 V and switched to 120 V after 30 min until the end of electrophoresis; for non-gradient gels, a constant voltage of 120 V was set until the end of electrophoresis.
(5) Staining: The gel was placed into a petri dish containing staining solution and stained on a shaker for approximately 15 minutes.
(6) Destaining: The gel was taken out of the staining solution, immersed in destaining solution, and placed on a shaker until the background became colorless.
(7) The samples were imaged with a Bio-Rad chemiluminescence imaging system, and relevant information was labeled.

The results of protein electrophoresis are shown in FIG. 1, where Lane M represents the protein Marker (Bio-Rad, Cat. no. 16103573S) with molecular weight references indicated by the annotations on the left; Lane R represents the result of the reduced sample, and Lane NR represents the result of the non-reduced sample. As shown in FIG. 1, the recombinantly prepared canine IL-31 protein met the required expression purity, with a molecular weight of 16.1965 kDa.

### 1.2. ELISA Assay

Commercially available anti-canine IL-31 caninized monoclonal antibody at 1 μg/mL was added to a 96-well plate at 100 μL/well and coated overnight at 4° C.; after washing the plate 3 times with 0.1% PBST washing buffer at 300 μL/well, blocking buffer was added and incubated at 37° C. for 1 h; after washing the plate 3 times, serially diluted IL-31 was added and incubated at 37° C. for 1 h; after washing the plate 3 times, HRP-labeled 6×His-IgG (H+L) (Abcam) diluted 1000-fold, 5000-fold and 10000-fold was tested and incubated at 37° C. for 1 h; after washing the plate 3 times, color development was performed with TMB chromogenic solution, the reaction was terminated with 1 M H₂SO₄ solution, and the absorbance was read at 450 nm using a microplate reader.

The results of the ELISA binding activity assay are presented in Table 2. As indicated by the data in Table 2, the recombinantly prepared canine IL-31 protein exhibited favorable binding activity to the caninized anti-canine IL-31 monoclonal antibody.

TABLE 2
ELISA Binding Activity Assay of Canine IL-31

| | OD₄₅₀ | | |
|---|---|---|---|
| Concentration of recombinant | HRP-labeled 6 × His-IgG (H + L) | | |
| canine IL-31 protein (ng/mL) | 1:1000 | 1:5000 | 1:10000 |
| 1000.000 | 3.041 | 2.686 | 2.390 |
| 333.333 | 3.022 | 2.736 | 2.406 |
| 111.111 | 3.043 | 2.733 | 2.437 |
| 37.037 | 2.862 | 2.558 | 2.139 |
| 12.346 | 2.525 | 2.063 | 1.585 |
| 0.000 | 0.045 | 0.047 | 0.058 |

### 2. Animal Immunization

In this example, a rapid immunization protocol (Phoenix MonRab) was adopted. Four New Zealand rabbits (designated R10265, R10266, R10267 and R10268, respectively) were immunized three times with recombinant canine IL-31 protein. Seven days after the third immunization, the sera of the immunized animals were detected by indirect ELISA to determine the level of the immune response. The ELISA detection procedure can be performed with reference to the experimental steps in Section 1.2. A 96-well plate was coated with 1 μg/mL recombinant canine IL-31 protein at 100 μL/well at 4° C. overnight. After washing three times with 0.1% PBST washing solution at 300 μL/well, blocking solution was added and the plate was incubated at 37° C. for 1 h. After washing three times, rabbit sera with different dilution ratios were added at 100 μL/well and the plate was incubated at 37° C. for 1 h. After washing three times, HRP-labeled goat anti-rabbit IgG (H+L) (diluted 1:20000) was added at 100 μL/well and the plate was incubated at 37° C. for 1 h. After washing three times, develop color with TMB chromogenic solution (100 μL per well); the reaction was terminated with 1 M H₂SO₄ solution, and the absorbance was read at 450 nm using a microplate reader.

The detection results are shown in Table 3. Finally, two rabbits, R10266 and R10267, were selected for B cell screening.

TABLE 3
Titer Detection of Animal Serum By Indirect ELISA

| | | Experimental group | | | | | | | | | | | | |
|---|---|---|---|---|---|---|---|---|---|---|---|---|---|---|
| | | 1 | 2 | 3 | 4 | 5 | 6 | 7 | 8 | 9 | 10 | 11 | | |
| | | Dilution ratio | | | | | | | | | | | | |
| | | 1:1000 | 1:2000 | 1:4000 | 1:8000 | 1:16000 | 1:32000 | 1:64000 | 1:128000 | 1:256000 | 1:512000 | Blank | Titer | Coating |
| Immu- | R10265 | 3.510 | 3.455 | 3.385 | 3.010 | 2.541 | 1.990 | 1.407 | 0.898 | 0.569 | 0.329 | 0.049 | 1:512000 | A |
| nized | | 1.320 | 0.645 | 0.358 | 0.187 | 0.124 | 0.088 | 0.062 | 0.051 | 0.062 | 0.045 | 0.045 | 1:16000 | B |
| ani- | R10266 | 3.291 | 3.325 | 3.680 | 3.533 | 3.213 | 2.982 | 2.695 | 2.283 | 1.707 | 1.148 | 0.046 | >:512000 | A |
| mals | | 3.433 | 3.103 | 2.790 | 2.210 | 1.606 | 1.120 | 0.685 | 0.394 | 0.243 | 0.159 | 0.046 | 1:512000 | B |
| | R10267 | 3.317 | 3.427 | 3.423 | 3.354 | 3.365 | 2.823 | 2.442 | 1.802 | 1.254 | 0.789 | 0.094 | >:512000 | A |
| | | 2.401 | 1.484 | 0.946 | 0.680 | 0.360 | 0.180 | 0.233 | 0.079 | 0.058 | 0.050 | 0.047 | 1:64000 | B |
| | R10268 | 3.431 | 3.467 | 3.444 | 3.232 | 2.977 | 2.423 | 1.935 | 1.348 | 0.812 | 0.486 | 0.046 | 1:512000 | A |
| | | 3.037 | 2.198 | 1.625 | 1.029 | 0.566 | 0.329 | 0.206 | 0.126 | 0.084 | 0.068 | 0.051 | 1:128000 | B |

Note:
The titer is the highest when (signal/blank) ≥ 2.1; the OD450 value of the blank is the average of two replicates.
A represents canine IL-31, and B represents an irrelevant negative protein with a His tag.

### 3. B Cell Clone Screening

### 3.1. PBMC Collection and B Cell Screening

Collect blood from rabbits with high immune response levels to harvest PBMCs and obtain antigen-positive B cells; plate the above-mentioned antigen-positive B cells into 96-well plates for screening.

### 3.2. Primary Screening

Collect the cell culture supernatants, screen the supernatants by the indirect ELISA method, and select the supernatants that are positive for canine IL-31 protein.

### 3.3. Confirmatory Screening

For all positive B cells obtained in the primary screening stage, screen all the positive cell supernatants with recombinant canine IL-31 protein by the indirect ELISA method, then perform counter-screening with an irrelevant His tag protein, and obtain clones targeting Group A (canine IL-31 protein positive) and Group B (irrelevant negative protein with a His tag). Table 4 shows the results of screening B cell supernatants by indirect ELISA; select the cell clones with high binding capacity to Group A and low binding capacity to Group B for subsequent experiments.

TABLE 4
Screening of B Cell Supernatants by Indirect ELISA

| | OD₄₅₀ | | | OD₄₅₀ | |
|---|---|---|---|---|---|
| Clone | A | B | Clone | A | B |
| 1F7 | 2.294 | 0.048 | 26D10 | 2.771 | 0.050 |
| 2B8 | 1.179 | 0.047 | 26A7 | 2.699 | 0.045 |
| 2H7 | 2.544 | 0.046 | 26F4 | 1.415 | 0.046 |
| 3E5 | 2.954 | 0.047 | 26H1 | 3.122 | 0.045 |
| 3F8 | 3.138 | 0.048 | 27C4 | 3.411 | 0.046 |
| 3H5 | 2.708 | 0.047 | 27D3 | 3.137 | 0.047 |
| 5A12 | 1.882 | 0.046 | 27D4 | 2.560 | 0.047 |
| 5C6 | 2.852 | 0.048 | 27D6 | 2.617 | 0.047 |
| 5C11 | 2.926 | 0.049 | 28B11 | 3.246 | 0.045 |
| 5H4 | 3.107 | 0.047 | 28C3 | 1.486 | 0.045 |
| 7G6 | 2.035 | 0.044 | 28D5 | 1.894 | 0.045 |
| 7H9 | 2.254 | 0.047 | 28E8 | 2.747 | 0.046 |
| 8B12 | 2.562 | 0.047 | 29D5 | 1.769 | 0.045 |
| 8C9 | 2.904 | 0.042 | 30A11 | 3.240 | 0.046 |
| 8G8 | 2.690 | 0.054 | 30G5 | 3.024 | 0.047 |
| 8H8 | 2.275 | 0.044 | 31A5 | 2.863 | 0.044 |
| 9B2 | 3.057 | 0.048 | 31A11 | 1.409 | 0.044 |
| 10H4 | 1.372 | 0.044 | 32A8 | 3.254 | 0.081 |
| 11C4 | 2.682 | 0.047 | 32B3 | 2.131 | 0.044 |
| 11C8 | 2.845 | 0.049 | 33B3 | 3.303 | 0.045 |
| 13G5 | 3.377 | 0.052 | 33C6 | 2.967 | 0.046 |
| 15G4 | 1.921 | 0.046 | 34E1 | 1.790 | 0.050 |
| 16A7 | 2.432 | 0.049 | 34H7 | 1.212 | 0.047 |
| 17D2 | 2.832 | 0.045 | 35C9 | 3.223 | 0.048 |
| 18E8 | 2.978 | 0.050 | 36D11 | 2.524 | 0.079 |
| 19C2 | 1.779 | 0.046 | 36H5 | 2.908 | 0.048 |
| 19C7 | 2.541 | 0.049 | 38A8 | 1.304 | 0.052 |
| 19D1 | 3.218 | 0.044 | 38E2 | 3.172 | 0.046 |
| 19H2 | 2.951 | 0.048 | 39C11 | 2.468 | 0.050 |
| 20C3 | 3.117 | 0.050 | 39G6 | 2.607 | 0.047 |
| 20E12 | 2.306 | 0.045 | 44B4 | 2.806 | 0.044 |
| 21E11 | 1.632 | 0.045 | 47C1 | 3.186 | 0.047 |
| 25A10 | 2.541 | 0.048 | CM | 0.063 | 0.062 |
| 25B10 | 3.340 | 0.052 | Irrelevant supernatant | 0.046 | 0.051 |
| 25C4 | 2.408 | 0.046 | Positive control | 3.104 | 1.932 |
| 25E12 | 2.558 | 0.047 | / | / | / |

### 3.4. Epitope Confirmation

Co-incubate the B cell culture supernatants with a commercially available caninized anti-canine IL-31 monoclonal antibody (positive control), and use a competitive ELISA to confirm whether the screened positive clones share the same epitope with the positive control. Among these, the results of epitope confirmation for the B cell culture supernatants are shown in Table 5-1 and Table 5-2; R10268 #1:100 represents the 100-fold dilution of the selected rabbit serum with the highest titer, which was used as the positive clone control.

The calculation formula for inhibition rate is: Inhibition rate (%)=100%−(OD450 of the clone itself/OD450 of blank medium)*100%

TABLE 5-1
Epitope Confirmation of B Cell Supernatants

| Caninized anti-canine IL-31 monoclonal | |
|---|---|
| antibody (positive control) | |
| 32 ng/mL | 16 ng/mL |

| | | Inhibition | | Inhibition |
|---|---|---|---|---|
| Clone | OD₄₅₀ | rate | OD₄₅₀ | rate |
| Blank Medium | 2.031 | 0.00% | 1.588 | 0.00% |
| R10268# 1:100 | 1.802 | 11.28% | 1.212 | 23.68% |
| 1F7 | 2.214 | −9.01% | 1.596 | −0.50% |
| 2B8 | 2.177 | −7.19% | 1.532 | 3.53% |
| 2H7 | 1.972 | 2.90% | 1.494 | 5.92% |
| 3E5 | 1.916 | 5.66% | 1.435 | 9.63% |
| 3F8 | 2.078 | −2.31% | 1.448 | 8.82% |
| 3H5 | 2.028 | 0.15% | 1.522 | 4.16% |
| 5A12 | 1.965 | 3.25% | 1.509 | 4.97% |
| 5C6 | 2.096 | −3.20% | 1.483 | 6.61% |
| 5C11 | 1.938 | 4.58% | 1.435 | 9.63% |
| 5H4 | 1.836 | 9.60% | 1.269 | 20.09% |
| 7G6 | 2.075 | −2.17% | 1.509 | 4.97% |
| 7H9 | 1.879 | 7.48% | 1.447 | 8.88% |
| 8B12 | 1.913 | 5.81% | 1.469 | 7.49% |
| 8C9 | 1.852 | 8.81% | 1.409 | 11.27% |
| 8G8 | 1.892 | 6.84% | 1.430 | 9.95% |
| 8H8 | 1.893 | 6.79% | 1.321 | 16.81% |
| 9B2 | 1.929 | 5.02% | 1.420 | 10.58% |
| 10H4 | 1.973 | 2.86% | 1.437 | 9.51% |
| 11C4 | 1.931 | 4.92% | 1.528 | 3.78% |
| 11C8 | 2.126 | −4.68% | 1.619 | −1.95% |
| 13G5 | 1.554 | 23.49% | 0.996 | 37.28% |
| 15G4 | 1.929 | 5.02% | 1.341 | 15.55% |
| 16A7 | 2.027 | 0.20% | 1.475 | 7.12% |
| 17D2 | 1.911 | 5.91% | 1.375 | 13.41% |
| 18E8 | 1.670 | 17.77% | 1.234 | 22.29% |
| 19C2 | 1.945 | 4.23% | 1.441 | 9.26% |
| 19C7 | 1.909 | 6.01% | 1.243 | 21.73% |
| 19D1 | 1.899 | 6.50% | 1.289 | 18.83% |
| 19H2 | 2.000 | 1.53% | 1.546 | 2.64% |
| 20C3 | 2.014 | 0.84% | 1.449 | 8.75% |
| 20E12 | 1.963 | 3.35% | 1.473 | 7.24% |
| 21E11 | 1.975 | 2.76% | 1.518 | 4.41% |
| 25A10 | 2.072 | −2.02% | 1.513 | 4.72% |
| 25B10 | 1.979 | 2.56% | 1.623 | −2.20% |
| 25C4 | 1.997 | 1.67% | 1.570 | 1.13% |
| 25E12 | 2.072 | −2.02% | 1.649 | −3.84% |
| 26D10 | 1.912 | 5.86% | 1.508 | 5.04% |
| 26A7 | 1.647 | 18.91% | 1.138 | 28.34% |
| 26F4 | 1.918 | 5.56% | 1.630 | −2.64% |
| 26H1 | 1.914 | 5.76% | 1.616 | −1.76% |
| 27C4 | 1.947 | 4.14% | 1.592 | −0.25% |

TABLE 5-2
Epitope Confirmation of B Cell Supernatants

| | Caninized anti-canine IL-31 monoclonal | | | |
|---|---|---|---|---|
| | antibody (positive control) | | | |
| | 32 ng/ml | | 16 ng/ml | |
| Clone | OD₄₅₀ | Inhibition rate | OD₄₅₀ | Inhibition rate |
| Blank medium | 2.054 | 0.00% | 1.592 | 0.00% |
| R10268# 1:100 | 1.939 | 5.60% | 1.357 | 14.76% |
| Irrelevant cell | 2.047 | 0.34% | 1.582 | 0.63% |
| supernatant | | | | |
| PC | 1.891 | 7.94% | 1.467 | 7.85% |
| 27D3 | 2.048 | 0.29% | 1.35 | 15.20% |
| 27D4 | 2.091 | −1.80% | 1.375 | 13.63% |
| 27D6 | 2.069 | −0.73% | 1.539 | 3.33% |
| 28B11 | 1.93 | 6.04% | 1.439 | 9.61% |
| 28C3 | 2.158 | −5.06% | 1.603 | −0.69% |
| 28D5 | 2.166 | −5.45% | 1.586 | 0.38% |
| 28E8 | 2.112 | −2.82% | 1.569 | 1.44% |
| 29D5 | 1.967 | 4.24% | 1.537 | 3.45% |
| 30A11 | 1.949 | 5.11% | 1.389 | 12.75% |
| 30G5 | 2.075 | −1.02% | 1.501 | 5.72% |
| 31A5 | 2.053 | 0.05% | 1.584 | 0.50% |
| 31A11 | 2.039 | 0.73% | 1.493 | 6.22% |
| 32A8 | 1.993 | 2.97% | 1.433 | 9.99% |
| 32B3 | 1.94 | 5.55% | 1.611 | −1.19% |
| 33B3 | 2.11 | −2.73% | 1.556 | 2.26% |
| 33C6 | 1.748 | 14.90% | 1.147 | 27.95% |
| 34E1 | 2.046 | 0.39% | 1.519 | 4.59% |
| 34H7 | 2.009 | 2.19% | 1.531 | 3.83% |
| 35C9 | 1.539 | 25.07% | 1.068 | 32.91% |
| 36D11 | 1.959 | 4.63% | 1.528 | 4.02% |
| 36H5 | 2.027 | 1.31% | 1.562 | 1.88% |
| 38A8 | 2.037 | 0.83% | 1.632 | −2.51% |
| 38E2 | 1.686 | 17.92% | 1.208 | 24.12% |
| 39C11 | 2.023 | 1.51% | 1.489 | 6.47% |
| 39G6 | 1.782 | 13.24% | 1.333 | 16.27% |
| 44B4 | 1.917 | 6.67% | 1.323 | 16.90% |
| 47C1 | 1.821 | 11.34% | 1.132 | 28.89% |

As shown in Table 5, B cell clones 5H4-2, 18E8-1, 19D1-1, 19C7-1, 30G5-1, 35C9-1, 38E2-1, 44B4-1, 47C1-1 and 30A11-1 exhibit good binding activity to canine IL-31 protein.

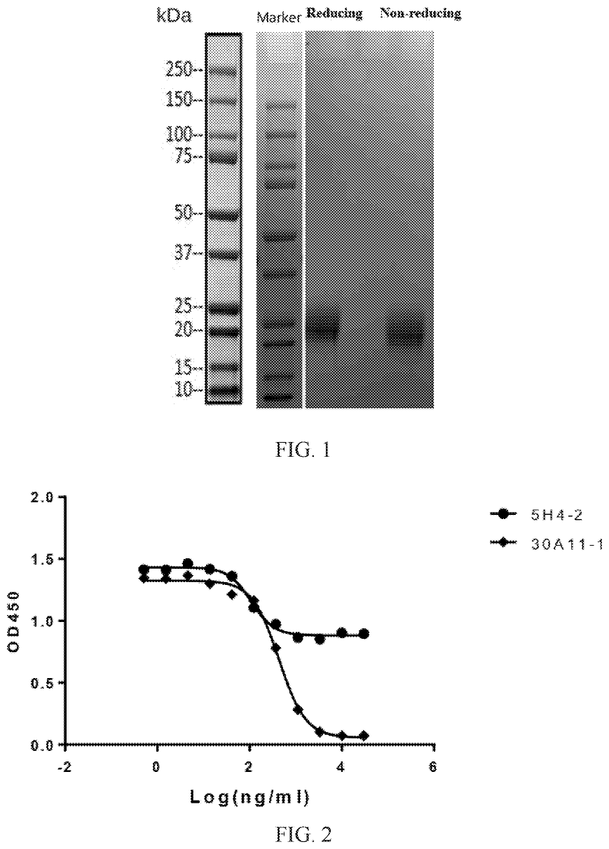

As shown in FIG. 2, 5H4-2 and 30A11-1 can compete with the positive control for binding to canine IL-31. Therefore, it is presumed that the B cell clones 5H4-2 and 30A11-1 may share the same epitope as the positive control.

### 3.5. ELISA Assay

ELISA assay was performed on the 10 B cell clones screened above with good binding ability to canine IL-31. The detailed procedure was as follows: canine IL-31 at 1 μg/mL was added to a 96-well plate at 100 μL/well and coated overnight at 4° C.; after washing the plate 3 times with 0.1% PBST washing buffer at 300 μL/well, blocking buffer was added and incubated at 37° C. for 1 h; after washing the plate 3 times, B cell culture supernatant was added and incubated at 37° C. for 1 h; after washing the plate 3 times, Goat-anti-Canine-IgG (H+L)-HRP was added and incubated at 37° C. for 1 h; after washing the plate 3 times, color development was performed with TMB chromogenic solution, the reaction was terminated with 1 M H₂SO₄ solution, and the absorbance was read at 450 nm using a microplate reader.

The binding activity of different cell samples is shown in Table 6. The results in Table 6 indicate that all cells show good binding activity. The binding activities of the 10 cells were ranked, among which B cell clone 19D1-1 exhibited the strongest binding activity.

TABLE 6
ELISA Assay Results

| | | | Binding activity |
|---|---|---|---|
| Experiment No. | Sample | EC₅₀ value/(ng/ml) | rank |
| 1 | 5H4-2 | 0.841 | 3 |
| 2 | 18E8-1 | 1.95 | 6 |
| 3 | 19D1-1 | 0.196 | 1 |
| 4 | 19C7-1 | 3.21 | 10 |
| 5 | 30G5-1 | 2.08 | 7 |
| 6 | 35C9-1 | 2.3 | 8 |
| 7 | 38E2-1 | 0.915 | 4 |
| 8 | 44B4-1 | 0.232 | 2 |
| 9 | 47C1-1 | 1.79 | 5 |
| 10 | 30A11-1 | 2.82 | 9 |

### 3.6. Cytobiological Activity Assay

As mentioned above, the pro-inflammatory cytokine interleukin 31 (IL-31) is produced by activated T lymphocytes in various species and induces pruritus in canine skin. In canine atopic dermatitis, when affected canines are exposed to allergens, T helper 2 (Th2) cells in the skin produce the cytokine IL-31. IL-31 binds to the co-receptor composed of IL-31 receptor A (IL-31RA) and oncostatin M receptor (OSMR), activates the STAT signaling cascade through Janus kinase phosphorylation, and leads to up-regulation of target genes, resulting in pruritic behavior in canines. Anti-IL-31 monoclonal antibodies can neutralize IL-31, thereby exerting an antipruritic effect.

To evaluate whether the 10 screened B cell clones can affect the ability of IL-31 to mediate pSTAT signaling, a cell cytobiological activity assay was established in this example. In this method, canine DH82 monocytes were pretreated with 10 ng/ml canine interferon-γ (R&D Systems, 781-CG-050) for 24 h and serum-starved for 2 h before IL-31 treatment to increase canine IL-31 receptor expression and pSTAT signal transduction. After pretreatment, recombinant canine IL-31 was added at 1 μg/mL for 5 min, and the STAT phosphorylation level was evaluated using HTRF technology (PerkinElmer, 62AT3PET). B cell supernatants were co-incubated with canine IL-31 for 1 h to qualitatively measure the ability of mAbs to inhibit STAT phosphorylation. Detailed experimental procedures are as follows:
(1) Cell sensitization: DH82 (canine macrophages/canine renal malignant histiocytosis cells) (Cell Bank of Chinese Academy of Sciences, Cat. No. TCO 3) were plated in a 96-well cell culture plate at 1×10⁵ cells/100 μL per well. The cells were cultured in MEM medium containing 15% FBS, 2 mmol/L GlutaMax and 1 mmol/L sodium pyruvate at 37° C. with 5% CO₂, and treated with 10 ng/mL canine interferon-γ for 24 h for sensitization.
(2) Washing: The medium was removed, and cells were washed once with 1×PBS at 200 μL/well.
(3) Serum starvation: The medium was replaced with serum-free medium at 100 μL/well, followed by 2 h of serum starvation.
(4) Sample treatment: IL-31 cytokine was diluted to 2 μg/mL with serum-free medium. B cell culture supernatant was mixed with IL-31 at equal volume and co-incubated for 1 h. Canine IL-31 at 1 μg/mL was used as the negative control.
(5) Addition of inhibitor and stimulant: After removing the medium, the pre-co-incubated mixture of mAb and IL-31 cytokine was added at 100 μL per well, and the cells were incubated for another 1 h at 37° C. in a CO₂ incubator.
(6) Washing: The medium was removed, and cells were washed once with 1×PBS at 200 μL/well.
(7) Cell lysis: 30 μL of 1× cell lysis buffer was added to each well, and lysis was performed at 100 rpm for 30 min. Upon completion of lysis, sample preparation was finished.
(8) Sample addition: 16 μL of cell lysis supernatant and 4 μL of pre-mixed antibody were added to the 96-well plate supplied with HTRF. The plate was incubated overnight at room temperature before reading. 16 μL of phosphorylation control and negative control were included. The 96-well plate was covered with sealing film.
(9) Incubation: Incubate overnight at room temperature.
(10) Reading: The reader was set to Eu*Cryptate mode, and fluorescence emissions at two different wavelengths (665 nm and 620 nm) were read in a compatible HTRF reader. The inhibition rate of the antibody was calculated.
(11) Data processing:

Ratio=(Signal at 665 nm/Signal at 620 nm)*10000

Inhibition rate=100%−(Sample Ratio/Canine IL-31 Negative Control Ratio)*100%

Assay establishment criterion: the value of phosphorylated control ratio/negative control ratio>2

### (12) Result Analysis

Upon calculation, the ratio of phosphorylated control to negative control in this assay was 3.8, indicating the assay was valid.

The cell activity assay results are shown in Table 7. As shown in Table 7, the B cell clones with an inhibition rate above 20% were 38E2-1, 47C1-1 and 35C9-1.

TABLE 7
Cytobiological Activity Assay

| No. | Clone No. | Inhibition rate % | Rank |
|---|---|---|---|
| 1 | 5H4-2 | 8.7 | 10 |
| 2 | 18E8-1 | 19.5 | 5 |
| 3 | 19D1-1 | 19.8 | 4 |
| 4 | 19C7-1 | 13.4 | 8 |
| 5 | 30G5-1 | 12 | 9 |
| 6 | 35C9-1 | 24.6 | 3 |
| 7 | 38E2-1 | 29.4 | 1 |
| 8 | 44B4-1 | 19.5 | 6 |
| 9 | 47C1-1 | 28 | 2 |
| 10 | 30A11-1 | 13.5 | 7 |

### 4. Antibody Sequencing

Perform V-region sequencing on the 10 ELISA-positive B cell clones by Sanger sequencing. The amino acid sequence numbers of the 10 antibodies are shown in Table 8, and the nucleotide sequence numbers are shown in Table 9.

TABLE 8
Information of Amino Acid Sequence Numbers of Antibodies

| | Amino acid sequence number of each antibody (SEQ ID NO: X) | | | | | | | | | |
|---|---|---|---|---|---|---|---|---|---|---|
| | 35C9-1 | 47C1-1 | 38E2-1 | 5H4-2 | 19D1-1 | 18E8-1 | 19C7-1 | 44B4-1 | 30A11-1 | 30G5-1 |
| HC- | 1 | 2 | 3 | 4 | 5 | 6 | 7 | 8 | 9 | 10 |
| CDR1 | | | | | | | | | | |
| HC- | 11 | 12 | 13 | 14 | 15 | 16 | 17 | 18 | 19 | 20 |
| CDR2 | | | | | | | | | | |
| HC- | 21 | 22 | 23 | 24 | 25 | 26 | 27 | 28 | 29 | 30 |
| CDR3 | | | | | | | | | | |
| LC- | 31 | 32 | 33 | 34 | 35 | 36 | 37 | 38 | 39 | 40 |
| CDR1 | | | | | | | | | | |
| LC- | 41 | 42 | 43 | 44 | 45 | 46 | 47 | 48 | 49 | 50 |
| CDR2 | | | | | | | | | | |
| LC- | 51 | 52 | 53 | 54 | 55 | 56 | 57 | 58 | 59 | 60 |
| CDR3 | | | | | | | | | | |
| VH | 61 | 62 | 63 | 64 | 65 | 66 | 67 | 68 | 69 | 70 |
| VL | 71 | 72 | 73 | 74 | 75 | 76 | 77 | 78 | 79 | 80 |

TABLE 9
Information of Nucleotide Sequence Numbers of Antibodies

| | Nucleotide sequence number of each antibody (SEQ ID NO: X) | | | | | | | | | |
|---|---|---|---|---|---|---|---|---|---|---|
| | 35C9-1 | 47C1-1 | 38E2-1 | 5H4-2 | 19D1-1 | 18E8-1 | 19C7-1 | 44B4-1 | 30A11-1 | 30G5-1 |
| HC- | 81 | 82 | 83 | 84 | 85 | 86 | 87 | 88 | 89 | 90 |
| CDR1 | | | | | | | | | | |
| HC- | 91 | 92 | 93 | 94 | 95 | 96 | 97 | 98 | 99 | 100 |
| CDR2 | | | | | | | | | | |
| HC- | 101 | 102 | 103 | 104 | 105 | 106 | 107 | 108 | 109 | 110 |
| CDR3 | | | | | | | | | | |
| LC- | 111 | 112 | 113 | 114 | 115 | 116 | 117 | 118 | 119 | 120 |
| CDR1 | | | | | | | | | | |
| LC- | 121 | 122 | 123 | 124 | 125 | 126 | 127 | 128 | 129 | 130 |
| CDR2 | | | | | | | | | | |
| LC- | 131 | 132 | 133 | 134 | 135 | 136 | 137 | 138 | 139 | 140 |
| CDR3 | | | | | | | | | | |
| VH | 141 | 142 | 143 | 144 | 145 | 146 | 147 | 148 | 149 | 150 |
| VL | 151 | 152 | 153 | 154 | 155 | 156 | 157 | 158 | 159 | 160 |

### 5. BLI Affinity Assay

### 5.1. Transient Expression of Rabbit-Derived Anti-IL-31 Monoclonal Antibody

### (1) Gene Sequence Design

Five clones, 38E2-1, 47C1-1, 35C9-1, 19D1-1 and 5H4-2, were selected from the 10 B cell clones. The heavy chain variable regions and light chain variable regions of these five molecules were spliced with the heavy chain constant regions and light chain constant regions of rabbit IgG, respectively, to obtain the complete sequences of the heavy and light chains of the five molecules.

### (2) Sequence Synthesis and Expression Vector

The fused heavy and light chains of the five molecules 38E2-1, 47C1-1, 35C9-1, 19D1-1 and 5H4-2 were synthesized separately. The genes were first amplified by PCR, then the pcDNA3.1 vector was linearized with restriction endonucleases NheI and ApaI. Finally, the genes were inserted into the NheI and Apal restriction sites of the pcDNA3.1 vector by seamless cloning. The ligation products were transformed into E. coli Top10 competent cells. Single colonies were picked after plating and verified by sequencing. Single colonies with successful transformation were expanded for endotoxin-free plasmid extraction and integrated into the pcDNA3.1 plasmid. Finally, plasmids 38E2-1-pcDNA3.1, 47C1-1-pcDNA3.1, 35C9-1-pcDNA3.1, 19D1-1-pcDNA3.1 and 5H4-2-pcDNA3.1 were obtained.

### (3) Transient Transfection of CHO Cells for Expression of Rabbit-Derived Anti-IL-31 Monoclonal Antibody

1) CHO-s cell culture: Under routine culture conditions in 125 mL shake flasks, when the cell density reached 1×10⁷ cells/mL, cells were seeded into new 125 mL shake flasks at an initial density of 2×10⁵ cells/mL and cultured at 37° C. with 5% CO₂. After 2 days, when the density reached 5×10⁵ cells/mL, transient transfection was performed according to the instructions of the transfection reagent Lipofectamine 2000;
2) Cell supernatants were collected after 7 days, centrifuged at 5000 rpm for 30 min to remove cells and debris, filtered through 0.45 μm filters, and stored temporarily at 4° C. for subsequent antibody purification.

### 5.2. Purification of Rabbit-Derived Anti-IL-31 Monoclonal Antibody

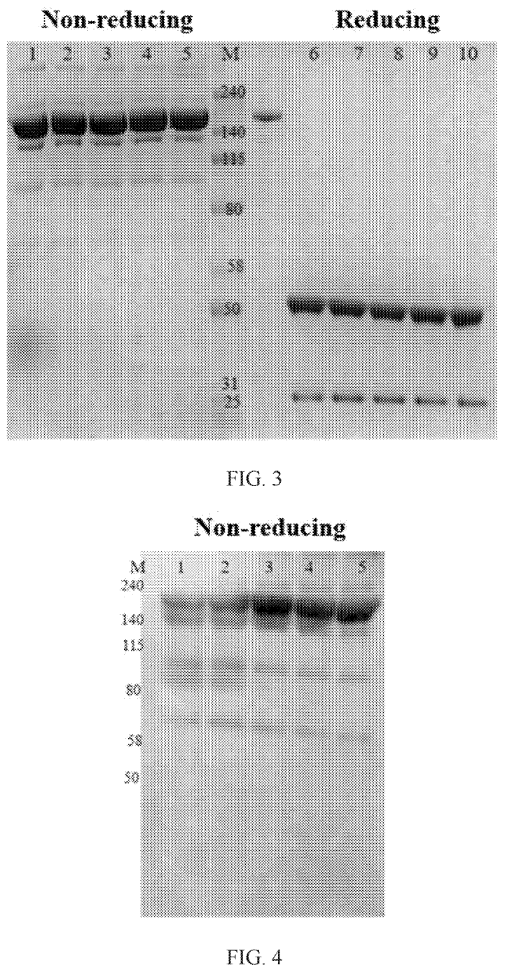

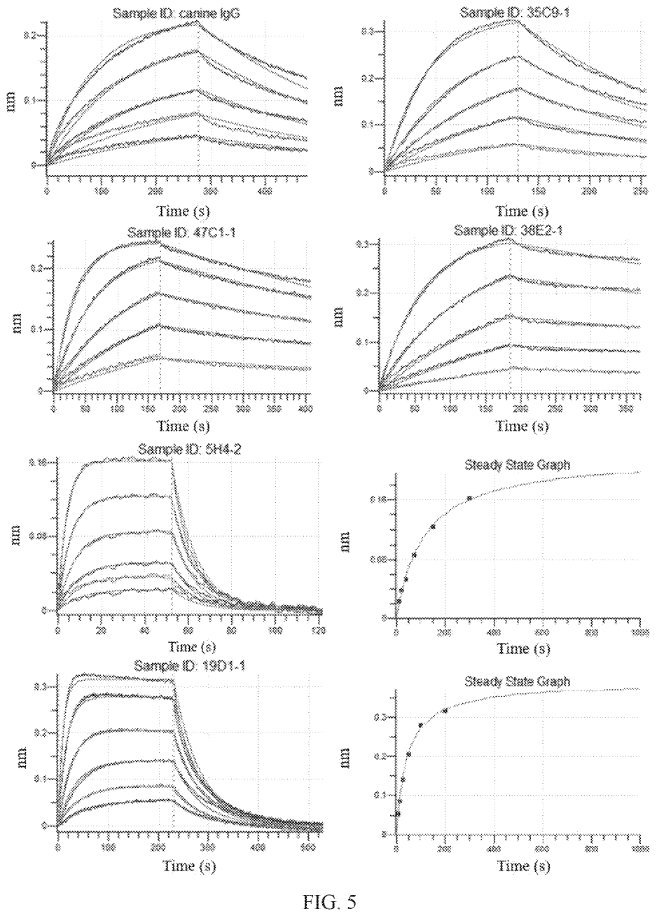

The cell culture supernatant was purified by Protein A affinity chromatography. The specific operation steps were: equilibration was performed with affinity chromatography equilibration solution (20 mM PB, 0.15 M NaCl, pH 7.2). After the clarified and filtered collection solution was loaded, equilibration was performed with affinity chromatography equilibration solution (20 mM PB, 0.15 M NaCl, pH 7.2). Then washing was performed with affinity chromatography washing solution (20 mM PB, 1 M NaCl, pH 7.2), and elution was performed with elution solution (0.1 M glycine-HCl, pH 3.0). The protein electrophoresis results were shown in FIG. 3. It could be seen from FIG. 3 that the SDS-PAGE purity of the eluted sample was more than 95%, and the target molecular weight was about 150 KD. The results of determining the rabbit-derived antibody candidates by Western Blot were shown in FIG. 4. It could be seen from FIG. 4 that the rabbit-derived antibody candidates could all bind to canine IL-31.

### 5.3. Affinity Assay

Five clones including 38E2-1, 47C1-1, 35C9-1, 19D1-1 and 5H4-2 were selected. A commercially available caninized anti-canine IL-31 monoclonal antibody was used as a positive control. The purified antibody was immobilized on a ProA biosensor, and canine IL-31 protein was used as the mobile phase to perform BLI affinity assay.

The affinity assay results were shown in Table 10. It could be seen from Table 10 that the affinity of three clones was higher than that of the positive control (Canine IgG), namely 38E2-1, 47C1-1 and 35C9-1. The affinity of 38E2-1 was the best, which was 2.547E-09. The BLI affinity assay results were shown in FIG. 5, which are consistent with the cytobiological activity assay results.

TABLE 10
BLI Affinity Assay

| Antibody | Analyte | Rmax | Ka(1/Ms) | Kdis(1/s) | KD(M) | Dissoc X² | Dissoc X² |
|---|---|---|---|---|---|---|---|
| 38E2-1 | Canine | 0.3318 | 3.302E−05 | 8.412E−04 | 2.547E−09 | 0.1513 | 0.998 |
| 47C1-1 | IL-31 | 0.2525 | 3.877E−05 | 1.457E−03 | 3.749E−09 | 0.1622 | 0.9964 |
| 35C9-1 | | 0.4167 | 3.995E−05 | 5.067E−03 | 1.268E−08 | 0.1769 | 0.9959 |
| Canine IgG | | 0.2956 | 9.597E−04 | 3.057E−03 | 3.186E−08 | 0.4687 | 0.9877 |
| 19D1-1 | | 0.3889 | N/A | N/A | 4.330E−08 | N/A | 0.9984 |
| 5H4-2 | | 0.2205 | N/A | N/A | 1.131E−07 | N/A | 0.9934 |

### Example 2: Establishment of a Canine Pruritus Model

### 1. Preliminary Establishment of a Canine Pruritus Model

To confirm the correlation between the inhibition of IL-31-mediated cellular signal transduction signals observed in the DH82 (canine renal malignant histiocytosis cell) assay and the inhibition of IL-31-mediated pruritus in dogs, the applicant referred to relevant literature and patents and established a canine pruritus model using recombinant canine IL-31 protein to evaluate the in vivo activity of the antibody. In this model, the recombinantly expressed canine IL-31 was administered to experimental canines via the intravenous route, with the low-dose group set at 3 μg/kg and the high-dose group at 8 μg/kg; sterile PBS was used as the negative control, and 5 experimental canines were selected for each group respectively. Clinical observation was initiated 30 min before administration and recorded continuously for 30 min; a second round of clinical observation was started 30 min after administration and recorded continuously for 2 h, and the pruritus frequency of the animals was recorded in both observation periods. The 30 min clinical observation before administration was used to establish a baseline. During clinical observation, animal behaviors such as licking, scratching, biting, pawing, rubbing, frequent head shaking, and digging were all identified as pruritic behaviors. Specifically, a “yes/no” judgment was made on whether each dog exhibited pruritic behaviors at consecutive 1-minute intervals. The occurrence of pruritic behavior—such as licking/chewing the paws, flanks, and/or perianal region, scratching the flanks, neck, and/or floor, shaking the head, or rubbing their buttocks on the cage floor—was sufficient to yield a “yes” response for the specified time interval. At the end of the period, the number of “yes” judgments was summed up to obtain the cumulative Pruritus Score Index (PSI). The PSI results for the cumulative 30 min before injection and the cumulative 2 h half an hour after injection were statistically analyzed; one-way analysis of variance in SPSS software was used to compare significant differences in PSI between each dose group and the negative control group respectively. When P>0.05, it indicates that there was no significant difference in PSI levels between each dose group and the control group; if P<0.05, it indicates that there were significant differences in PSI levels between each dose group and the control group.

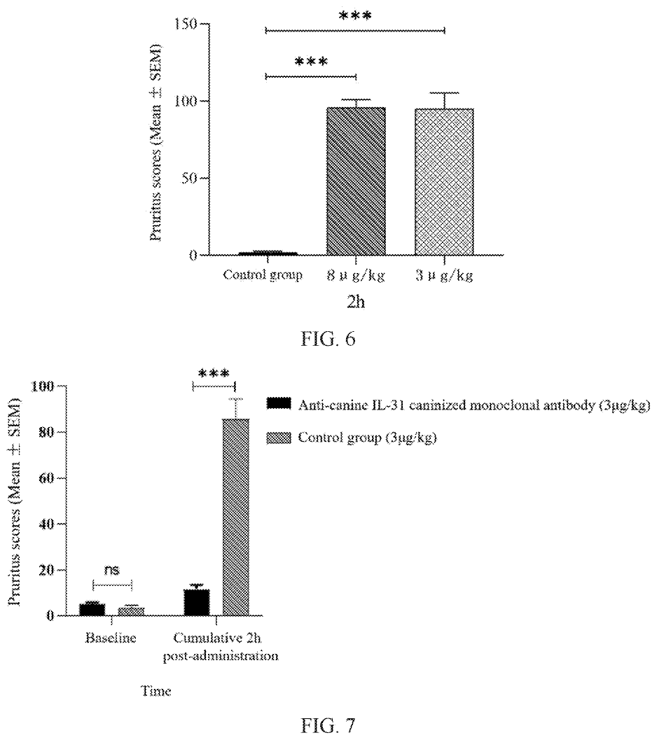

The results of the statistical analysis are shown in FIG. 6. It could be seen from FIG. 6 that the PSI results for the cumulative 2 h half an hour after injection in both the high-dose group and the low-dose group showed significant differences compared with the negative control, and there was no significant difference in the PSI results between the high-dose group and the low-dose group. A dose of 3 μg/kg was initially selected for establishing the canine pruritus model. 2. Pruritus Model Confirmation Experiment

After determining the intravenous administration dose of canine IL-31, we further confirmed the model. On Day-7 (D-7), canine IL-31 was administered to experimental canines via the intravenous route at a dose of 3 μg/kg, with sterile PBS used as the negative control; 10 experimental canines that showed significant differences from the negative control were selected. On Day 0 (D0), experimental canines in the treatment groups (5 canines/group) were subcutaneously injected with 2 mg/kg of a commercially available caninized anti-canine IL-31 monoclonal antibody via the cervical region, while experimental canines in the control group (5 canines/group) were subcutaneously injected with an equal volume of sterile PBS via the cervical region. On Day 8 (D8), 3 μg/kg of canine IL-31 was administered to experimental canines via the intravenous route. Clinical observation was initiated 30 min before administration and recorded continuously for 30 min; clinical observation was started again 30 min after administration and recorded continuously for 2 h, with the pruritus frequency of the animals recorded in both observation periods. The 30 min clinical observation before administration was used to establish a baseline. The PSI results for the cumulative 30 min before injection and the cumulative 2 h half an hour after injection were statistically analyzed; one-way analysis of variance in SPSS software was used to compare the significant differences in PSI between the treatment groups and the control group, respectively. When P>0.05, it indicated that there was no significant difference in PSI levels between the treatment groups and the control group; if P<0.05, it indicated that there were significant differences in PSI levels between the treatment groups and the control group.

The statistical results are shown in FIG. 7. As could be seen from FIG. 7, there was a significant difference between the treatment groups and the control group after D8, indicating that the caninized anti-canine IL-31 monoclonal antibody was able to inhibit IL-31-mediated pruritic symptoms in dogs 8 days after administration.

Ultimately, a canine pruritus model was successfully established by intravenously administering canine IL-31, and the intravenous administration dose of canine IL-31 was determined to be 3 μg/kg.

### Example 3: In vivo Efficacy Study of Rabbit-Derived Anti-IL-31 Monoclonal Antibodies

After the establishment of the canine pruritus model, the applicant further investigated the in vivo efficacy of rabbit-derived anti-IL-31 monoclonal antibodies. A certain amount of rabbit-derived antibody samples 38E2-1, 47C1-1 and 35C9-1 were transiently expressed. A commercially available caninized anti-canine IL-31 monoclonal antibody was used as the positive control, and sterile PBS was used as the negative control. 5 experimental canines were selected for each group, requiring a total of 25 experimental canines. Abnormal experimental canines were eliminated via the canine pruritus model. On Day 0 (D0), experimental canines in the treatment groups (5 canines/group) were subcutaneously injected with rabbit-derived antibody 38E2-1, rabbit-derived antibody 47C1-1 and rabbit-derived antibody 35C9-1 via the cervical region respectively, with the injection dose of each being 2 mg/kg; experimental canines in the positive control group (5 canines/group) were subcutaneously injected with 2 mg/kg of the caninized anti-canine IL-31 monoclonal antibody via the cervical region; experimental canines in the negative control group (5 canines/group) were subcutaneously injected with an equal volume of sterile PBS via the cervical region. With reference to relevant literatures, 3 μg/kg of canine IL-31 was administered to experimental canines via the intravenous route after 2.5 h. Clinical observation was initiated 30 min before administration and recorded continuously for 30 min; clinical observation was started again 30 min after administration and recorded continuously for 2 h, with the pruritus frequency of the animals recorded in both observation periods. The 30 min clinical observation before administration was used to establish a baseline. The PSI results for the cumulative 30 min before injection and the cumulative 2 h half an hour after injection were statistically analyzed; one-way analysis of variance in SPSS software was used to compare the significant differences in PSI between the treatment groups and the control group, respectively. When P>0.05, it indicated that there was no significant difference in PSI levels between the treatment groups and the control group; if P<0.05, it indicated that there were significant differences in PSI levels between the treatment groups and the control group.

The experimental results are shown in FIG. 8. As could be seen from FIG. 8, there were significant differences between the treatment groups and the negative control group (sterile PBS) for the cumulative 2 h half an hour after injection. For rabbit-derived antibody 38E2-1 compared with the positive control group, P>0.05, indicating no significant difference; for rabbit-derived antibody 47C1-1 compared with the positive control group, P>0.05, indicating no significant difference; for rabbit-derived antibody 35C9-1 compared with the positive control group, P=0.00014, indicating a significant difference. Among the in vivo activities of the three rabbit-derived antibodies, 38E2-1 exhibited the best efficacy, followed by 47C1-1 and 35C9-1.

### Example 4: Caninized Antibody Engineering Strategy

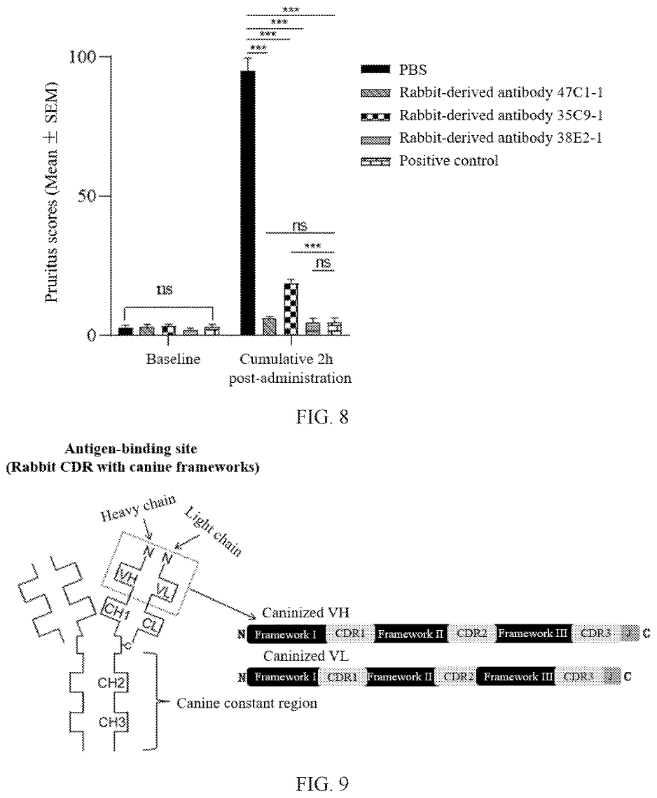

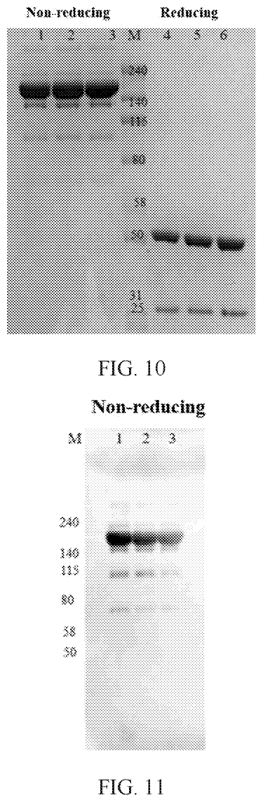

The production of anti-drug antibodies (ADAs) may be associated with the loss of efficacy of any biotherapeutic protein including monoclonal antibodies. To help mitigate the risks associated with the formation of ADAs against the rabbit anti-IL-31 monoclonal antibodies provided herein, a caninization strategy was proposed to construct chimeric antibodies. See FIG. 9; this caninization strategy is based on identifying the canine germline antibody sequences most suitable for CDR grafting. After an extensive analysis of the heavy and light chains of all available canine germline sequences, candidate germlines were selected based on their homology to the rabbit mAbs. The specific steps were as follows: a caninized template with the highest degree of matching to the FV regions of the rabbit-derived antibodies was retrieved from the IMGT website; the CDR regions in the caninized template were replaced with the CDR regions of rabbit; the sequences were analyzed using bioinformatics software; and back mutations were performed on several amino acids. It was expected to obtain several caninized antibodies with affinity and activity comparable to those of the parental antibodies.

### Example 5: Construction and Preparation of Chimeric Antibodies 38E2-1, 47C1-1 and 35C9-1

### 1. Construction and Transient Expression of Chimeric Antibodies 38E2-1, 47C1-1 and 35C9-1

In accordance with the caninization engineering strategy described in Example 4, the heavy chain variable regions and light chain variable regions of the three molecules (38E2-1, 47C1-1 and 35C9-1) were spliced to the heavy chain constant region and light chain constant region of canine IgGB, respectively, to obtain the complete heavy and light chain sequences of these three molecules. The amino acid sequences and DNA sequences of the heavy chain and light chain of canine IgGB are available from the GenBank database. The GenBank accession number for the amino acid sequence of the canine IgGB heavy chain is AAL35302.1 (SEQ ID NO: 161), and that for DNA sequence is AF354265.1 (SEQ ID NO: 162); the GenBank accession number for the amino acid sequence of the canine antibody k light chain is ABY57289.1, and that for DNA sequence is EU305402.1. The amino acid and nucleotide sequence number information for the chimeric antibodies is shown in Table 11.

TABLE 11
Information of Sequence Number of The Chimeric Antibodies

| | | Chimeric antibodies | | |
|---|---|---|---|---|
| | | 38E2-1 | 47C1-1 | 35C9-1 |
| Amino acid | HC-CDR1 | 3 | 2 | 1 |
| sequence number | HC-CDR2 | 13 | 12 | 11 |
| (SEQ ID NO: X) | HC-CDR3 | 23 | 22 | 21 |
| | Heavy chain | 163 | 164 | 165 |
| | LC-CDR1 | 33 | 32 | 31 |
| | LC-CDR2 | 43 | 42 | 41 |
| | LC-CDR3 | 53 | 52 | 51 |
| | Light chain | 166 | 167 | 168 |
| Nucleotide | HC-CDR1 | 83 | 82 | 81 |
| sequence number | HC-CDR2 | 93 | 92 | 91 |
| (SEQ ID NO: X) | HC-CDR3 | 103 | 102 | 101 |
| | Heavy chain | 169 | 170 | 171 |
| | LC-CDR1 | 113 | 112 | 111 |
| | LC-CDR2 | 123 | 122 | 121 |
| | LC-CDR3 | 133 | 132 | 131 |
| | Light chain | 172 | 173 | 174 |

Sequence synthesis and transient transfection expression in CHO cells were performed in accordance with the method described in Example 1.5, and one-step affinity purification was conducted to evaluate the purity of the chimeric antibodies. The experimental results are shown in FIG. 10. As could be seen from FIG. 10, the purity of the chimeric antibodies determined by SDS-PAGE was over 95%, with the target molecular weight at approximately 150 kDa. The results of Western Blot assay are shown in FIG. 11. As could be seen from FIG. 11, all chimeric antibody candidates were able to bind to canine IL-31.

### 2. Affinity Evaluation of Chimeric Antibodies

### 2.1. ELISA Assay

1 μg/mL canine IL-31 was added to a 96-well plate at 100 L/well and coated overnight at 4° C.; after washing the plate 3 times with 0.1% PBST washing buffer at 300 μL/well, blocking buffer was added and incubated at 37° C. for 1 h; after washing the plate 3 times, the serially diluted chimeric antibody was added and incubated at 37° C. for 1 h; after washing the plate 3 times, Goat-anti-Canine-IgG (H+L)-HRP was added and incubated at 37° C. for 1 h; after washing the plate 3 times, color development was performed with TMB chromogenic solution, the reaction was terminated with 1 M H₂SO₄ solution, and the absorbance was read at 450 nm using a microplate reader.

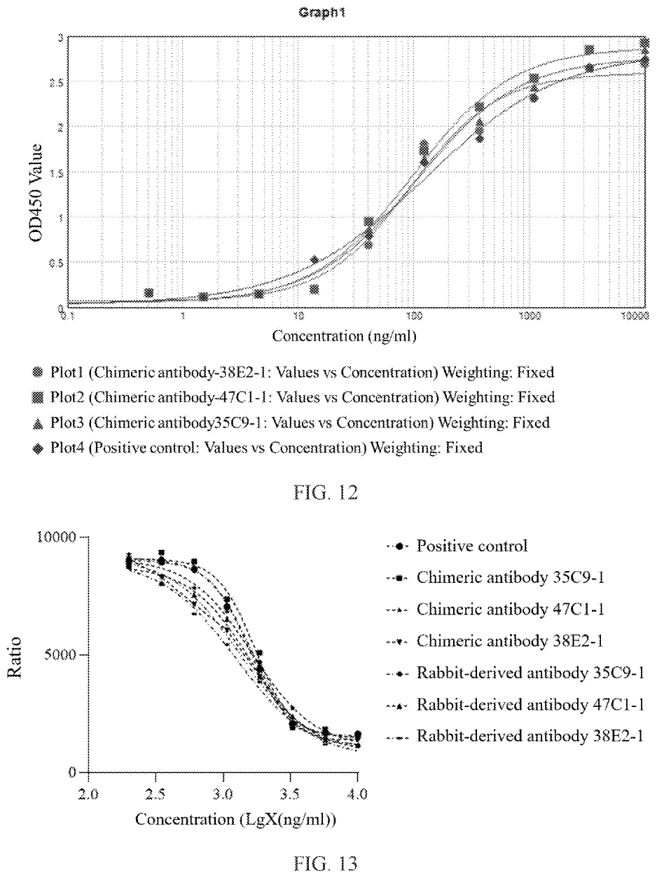

The detection results are shown in Table 12 and FIG. 12. As could be seen from Table 12, the binding activities of the three chimeric antibodies to canine IL-31 were higher than that of the positive control, with 38E2-1 the best, followed by 47C1-1, and 35C9-1 the weakest.

TABLE 12
ELISA Assay of Chimeric Antibodies

| | | EC₅₀ Value |
|---|---|---|
| No. | Antibody | (ng/mL) |
| 1 | Chimeric Antibody 38E2-1 | 93.99 |
| 2 | Chimeric Antibody 35C9-1 | 105.50 |
| 3 | Chimeric Antibody 47C1-1 | 98.45 |
| 4 | Anti-Canine IL-31 Caninized Monoclonal | 125.70 |
| | Antibody Positive Control | |

### 2.2. BLI Affinity Assay

The purified antibody was immobilized on a ProA biosensor, and canine IL-31 protein was used as the mobile phase to perform BLI affinity assay. As shown in Table 13, the affinity of the chimeric antibodies was not different or slightly reduced (within 3-fold) compared with the corresponding rabbit-derived antibodies, and all were higher than that of the positive control.

TABLE 13
BLI Affinity Assay of Chimeric Antibodies

| | Affinity assay KD |
|---|---|
| Antibody | (M) |
| Rabbit-derived Antibody 38E2-1 | 8.15E−10 |
| Chimeric Antibody 38E2-1 | 2.49E−09 |
| Rabbit-derived Antibody 47C1-1 | 3.75E−09 |
| Chimeric Antibody 47C1-1 | 8.32E−09 |
| Rabbit-derived Antibody 35C9-1 | 1.27E−08 |
| Chimeric Antibody 35C9-1 | 1.19E−08 |
| Anti-Canine IL-31 Caninized Monoclonal | 3.19E−08 |
| Antibody Positive Control | |

### 3. Evaluation of Cytobiological Activity of Chimeric Antibodies

### 3.1. In Vitro Activity Evaluation

The initial concentration of the purified chimeric antibody samples was diluted to 10 μg/mL, followed by 1.75-fold serial dilution to obtain a total of 8 concentration points. Each serially diluted sample was mixed with IL-31 protein in equal volume and co-incubated for 1 h. The in vitro activity was evaluated in accordance with the cytobiological activity assay described in Example 1.6. The detection results are shown in Table 14 and FIG. 13. As indicated by the above results, all three chimeric antibodies exhibited in vitro activity; among them, antibody 38E2-1 had a smaller IC₅₀ value and higher inhibition rate, showing the best in vitro activity.

TABLE 14
Cytobiological Activity Assay of Chimeric Antibodies

| | | IC₅₀ Value |
|---|---|---|
| No. | Antibody | (ng/mL) |
| 1 | Rabbit-derived Antibody 38E2-1 | 1250 |
| 2 | Chimeric Antibody 38E2-1 | 1323 |
| 3 | Rabbit-derived Antibody 47C1-1 | 1569 |
| 4 | Chimeric Antibody 47C1-1 | 1630 |
| 5 | Rabbit-derived Antibody 35C9-1 | 1638 |
| 6 | Chimeric Antibody 35C9-1 | 1714 |
| 7 | Anti-Canine IL-31 Caninized Monoclonal | 1548 |
| | Antibody Positive Control | |

### Example 6: In Vivo Pharmacodynamic Evaluation of Chimeric Antibodies

The in vivo efficacy of the chimeric antibodies was evaluated in accordance with the method described in Example 3. The applicant transiently expressed a certain amount of samples to assess the in vivo efficacy of chimeric antibodies 38E2-1, 47C1-1 and 35C9-1. A commercially available caninized anti-canine IL-31 monoclonal antibody was used as the positive control, and sterile PBS was used as the negative control. 5 experimental canines were selected for each group, requiring a total of 25 experimental canines. Abnormal experimental canines were eliminated via the canine pruritus model. On Day 0 (D0), experimental canines in the treatment groups (5 canines/group) were subcutaneously injected with chimeric antibody 38E2-1, chimeric antibody 47C1-1 and chimeric antibody 35C9-1 via the cervical region respectively, with the injection dose of each being 2 mg/kg; experimental canines in the positive control group (5 canines/group) were subcutaneously injected with 2 mg/kg of the caninized anti-canine IL-31 monoclonal antibody via the cervical region; experimental canines in the negative control group (5 canines/group) were subcutaneously injected with an equal volume of sterile PBS via the cervical region. With reference to relevant literatures, 3 μg/kg of canine IL-31 was administered to experimental canines via the intravenous route after 2.5 h. Clinical observation was initiated 30 min before administration and recorded continuously for 30 min; clinical observation was started again 30 min after administration and recorded continuously for 2 h, with the pruritus frequency of the animals recorded in both observation periods. The 30 min clinical observation before administration was used to establish a baseline. The PSI results for the cumulative 30 min before injection and the cumulative 2 h half an hour after injection were statistically analyzed; one-way analysis of variance in SPSS software was used to compare the significant differences in PSI between the treatment groups and the control group, respectively. When P>0.05, it indicated that there was no significant difference in PSI levels between the treatment groups and the control group; if P<0.05, it indicated that there were significant differences in PSI levels between the treatment groups and the control group.

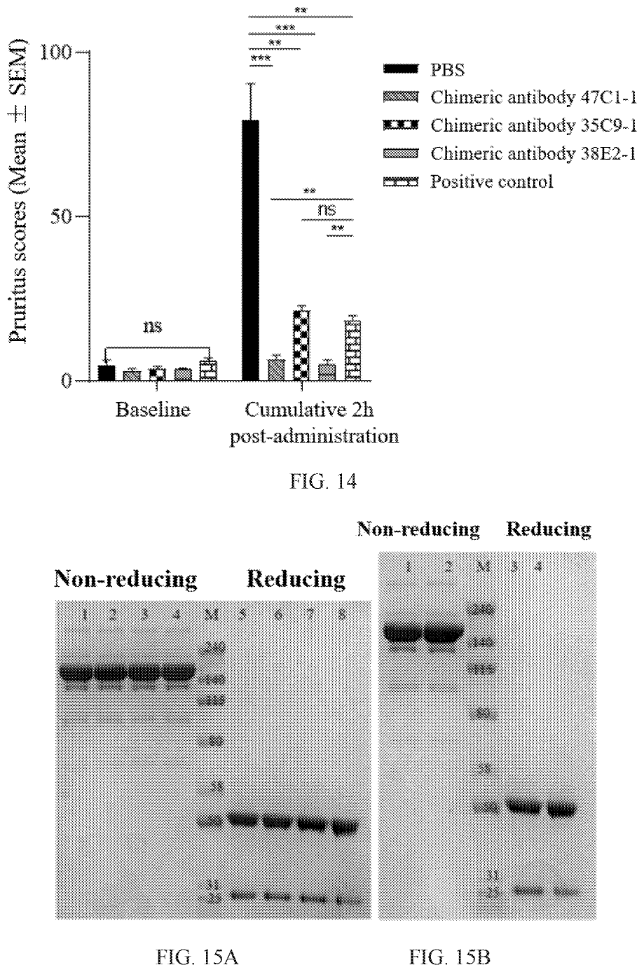

The statistical results of the experiment are shown in FIG. 14. As could be seen from FIG. 14, there were significant differences between the treatment groups and the negative control group. For chimeric antibody 38E2-1 compared with the positive control group, P=0.000302, indicating a significant difference. For chimeric antibody 35C9-1 compared with the positive control group, P>0.05, indicating no significant difference. For chimeric antibody 47C1-1 compared with the positive control group, P=0.000596, indicating a significant difference. Among the in vivo activities of the three chimeric antibodies, chimeric antibody 38E2-1 exhibited the best efficacy, followed by chimeric antibody 47C1-1 and chimeric antibody 35C9-1.

### Example 7: Construction and Preparation of Caninized Antibodies

### 1. Construction and Transient Expression of Caninized Antibodies 38E2-1, 47C1-1 and 35C9-1

Caninization of 38E2-1, 47C1-1 and 35C9-1 was performed in accordance with the caninization engineering strategy described in Example 4, and two caninized antibodies were obtained from each parental antibody, respectively, the sequence number information for the caninized antibodies is shown in Table 15.

TABLE 15
The sequence number information of the caninized antibodies

| | | Caninized antibodies | | | | | |
|---|---|---|---|---|---|---|---|
| | | 38E2-1- | 38E2-1- | 47C1-1- | 47C1-1- | 35C9-1- | 35C9-1- |
| | | H3L3 | H3L2 | H2L2 | H3L3 | H1L1 | H1L2 |
| Amino acid | HC-CDR1 | 3 | 3 | 2 | 2 | 1 | 1 |
| sequence | HC-CDR2 | 13 | 13 | 12 | 12 | 11 | 11 |
| number | HC-CDR3 | 23 | 23 | 22 | 22 | 21 | 21 |
| (SEQ ID | Heavy chain | 175 | 176 | 177 | 178 | 179 | 180 |
| NO: X) | LC-CDR1 | 33 | 33 | 32 | 32 | 31 | 31 |
| | LC-CDR2 | 43 | 43 | 42 | 42 | 41 | 41 |
| | LC-CDR3 | 53 | 53 | 52 | 52 | 51 | 51 |
| | Light chain | 181 | 182 | 183 | 184 | 185 | 186 |
| Nucleotide | HC-CDR1 | 83 | 83 | 82 | 82 | 81 | 81 |
| sequence | HC-CDR2 | 93 | 93 | 92 | 92 | 91 | 91 |
| number | HC-CDR3 | 103 | 103 | 102 | 102 | 101 | 101 |
| (SEQ ID | Heavy chain | 187 | 188 | 189 | 190 | 191 | 192 |
| NO: X) | LC-CDR1 | 113 | 113 | 112 | 112 | 111 | 111 |
| | LC-CDR2 | 123 | 123 | 122 | 122 | 121 | 121 |
| | LC-CDR3 | 133 | 133 | 132 | 132 | 131 | 131 |
| | Light chain | 193 | 194 | 195 | 196 | 197 | 198 |

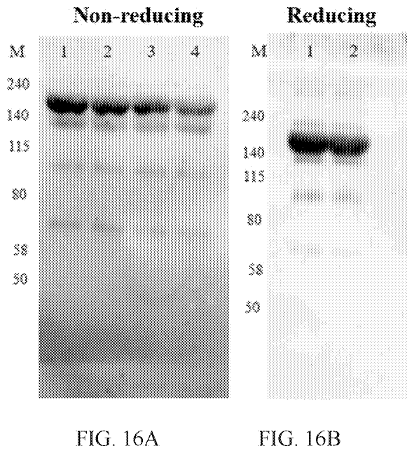

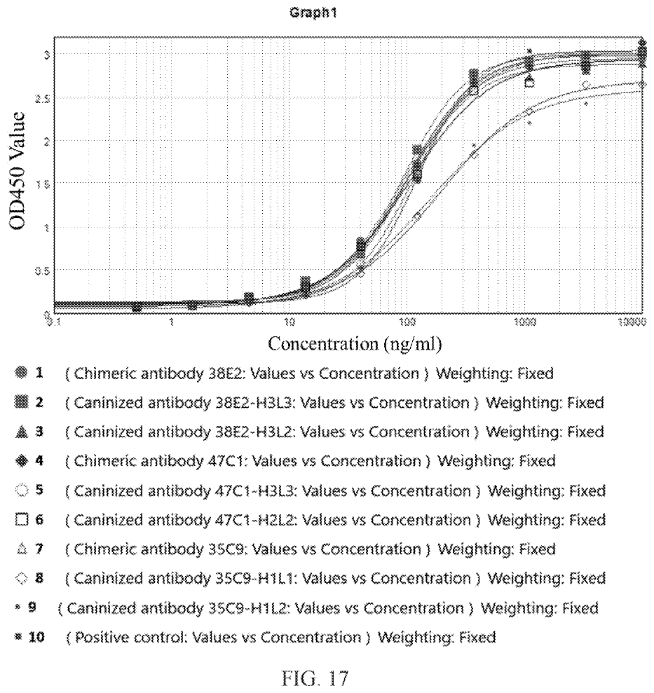

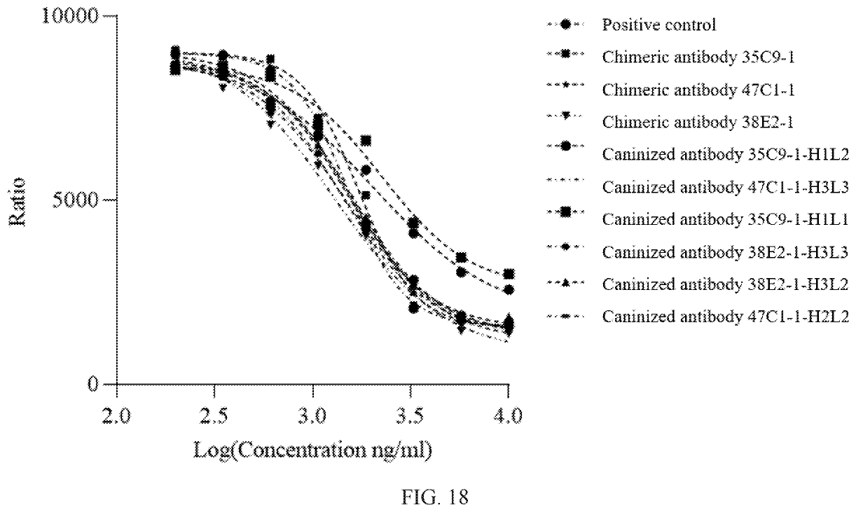

Sequence synthesis and transient transfection expression in CHO cells were performed in accordance with the method described in Example 1.5, and one-step affinity purification was conducted to evaluate the purity of the chimeric antibodies. The experimental results are shown in FIG. 15. As could be seen from FIG. 15, the purity of the chimeric antibodies determined by SDS-PAGE was over 95%, with the target molecular weight at approximately 150 kDa. The results of Western Blot assay are shown in FIG. 16. As could be seen from FIG. 16, all chimeric antibody candidates were able to bind to canine IL-31.

### 2. Affinity Evaluation of Caninized Antibodies

### (1) ELISA Assay

The caninized antibodies were determined in accordance with the ELISA method described in Example 4.2. The detection results are shown in FIG. 17 and Table 16. As indicated by the above results, the binding activities of the six caninized antibodies to canine IL-31 were slightly lower than those of the corresponding chimeric antibodies. The binding activities of the caninized antibodies 38E2-1 and 47C1-1 were not significantly different from those of the positive control, while the binding activities of the caninized antibodies 35C9-1-HIL1 and 35C9-1-H1L2 were inferior to that of the positive control.

TABLE 16
ELISA Assay of Caninized Antibodies

| | ELISA |
|---|---|
| Sample Name | (EC₅₀ Value, ng/ml) |
| Chimeric Antibody 38E2-1 | 92.85 |
| Caninized Antibody 38E2-1-H3L3 | 93.41 |
| Caninized Antibody 38E2-1-H3L2 | 97.75 |
| Chimeric Antibody 47C1-1 | 102.2 |
| Caninized Antibody 47C1-1-H2L2 | 113.4 |
| Caninized Antibody 47C1-1-H3L3 | 105.1 |
| Chimeric Antibody 35C9-1 | 102.7 |
| Caninized Antibody 35C9-1-H1L1 | 189.7 |
| Caninized Antibody 35C9-1-H1L2 | 157.6 |
| Anti-Canine IL-31 Caninized Monoclonal | 123.8 |
| Antibody Positive Control | |

### (2) BLI Affinity Assay

The caninized antibodies were determined in accordance with the BLI affinity assay method described in Example 4.2. As indicated by the results in Table 17, the affinity of the caninized antibodies was reduced by one order of magnitude compared with the rabbit-derived antibodies. However, the affinities of the modified 38E2-1 and 47C1-1 are still higher than that of the positive control, while the affinity of the modified 35C9-1 is not as good as that of the positive control.

TABLE 17
Affinity Assay of Caninized Antibodies

| | Affinity Assay KD |
|---|---|
| Name | (M) |
| Rabbit-derived Antibody 38E2-1 | 8.15E−10 |
| Chimeric Antibody 38E2-1 | 2.49E−09 |
| Caninized Antibody 38E2-1-H3L3 | 5.35E−09 |
| Caninized Antibody 38E2-1-H3L2 | 8.04E−09 |
| Rabbit-derived Antibody 47C1-1 | 3.75E−09 |
| Chimeric Antibody 47C1-1 | 8.32E−09 |
| Caninized Antibody 47C1-1-H2L2 | 1.51E−08 |
| Caninized Antibody 47C1-1-H3L3 | 1.55E−08 |
| Rabbit-derived Antibody 35C9-1 | 1.27E−08 |
| Chimeric Antibody 35C9-1 | 1.19E−08 |
| Caninized Antibody 35C9-1-H1L1 | 4.95E−07 |
| Caninized Antibody 35C9-1-H1L2 | 3.89E−07 |
| Anti-Canine IL-31 Caninized Monoclonal | 3.19E−08 |
| Antibody Positive Control | |

### 3. Evaluation of Cytobiological Activity of Caninized Antibodies

### (1) In Vitro Activity Evaluation

The purified caninized antibody samples were evaluated for in vitro activity in accordance with Example 4.3, and the results are shown in Table 18 and FIG. 18. As indicated by the above results, there was no significant difference between the caninized 38E2-1, 47C1-1 and their corresponding chimeric antibodies as well as the positive control. However, the cell activity of the caninized 35C9-1 decreased and was inferior to that of the positive control.

TABLE 18
Results of Cytobiological Activity Assay of Caninized Antibodies

| | In vitro Activity |
|---|---|
| Sample Name | (IC₅₀ Value ng/mL) |
| Chimeric Antibody 38E2 | 1296 |
| Caninized Antibody 38E2-1-H3L3 | 1397 |
| Caninized Antibody 38E2-1-H3L2 | 1500 |
| Chimeric Antibody 47C1 | 1558 |
| Caninized Antibody 47C1-1-H2L2 | 1448 |
| Caninized Antibody 47C1-1-H3L3 | 1612 |
| Chimeric Antibody 35C9 | 1711 |
| Caninized Antibody 35C9-1-H1L1 | 2256 |
| Caninized Antibody 35C9-1-H1L2 | 2045 |
| Anti-Canine IL-31 Caninized Monoclonal | 1508 |
| Antibody Positive Control | |

### Example 8: In Vivo Pharmacodynamic Evaluation of Caninized Antibodies

### 1. In Vivo Activity Evaluation of Caninized Antibodies

The in vivo pharmacodynamic evaluation of caninized antibodies was performed in accordance with the method described in Example 3. A certain amount of samples were transiently expressed to assess the in vivo efficacy of caninized antibodies 38E2-1, 47C1-1 and 35C9-1. A commercially available caninized anti-canine IL-31 monoclonal antibody was used as the positive control, and sterile PBS was used as the negative control. 5 experimental canines were selected for each group, requiring a total of 40 experimental canines. Abnormal experimental canines were eliminated via the canine pruritus model. On Day 0 (D0), experimental canines in the treatment groups (5 canines/group) were subcutaneously injected with caninized antibodies 38E2-1-H3L3, 38E2-1-H3L2, 47C1-1-H2L2, 47C1-1-H3L3, 35C9-1-H1L1 and 35C9-1-H1L2 via the cervical region respectively, with the injection dose of each being 2 mg/kg; experimental canines in the positive control group (5 canines/group) were subcutaneously injected with 2 mg/kg of the caninized anti-canine IL-31 monoclonal antibody via the cervical region; experimental canines in the negative control group (5 canines/group) were subcutaneously injected with an equal volume of sterile PBS via the cervical region. With reference to relevant literatures, 3 g/kg of canine IL-31 was administered to experimental canines via the intravenous route after 2.5 h. Clinical observation was initiated 30 min before administration and recorded continuously for 30 min; clinical observation was started again 30 min after administration and recorded continuously for 2 h, with the pruritus frequency of the animals recorded in both observation periods. The 30 min clinical observation before administration was used to establish a baseline. The PSI results for the cumulative 30 min before injection and the cumulative 2 h half an hour after injection were statistically analyzed; one-way analysis of variance in SPSS software was used to compare the significant differences in PSI between the treatment groups and the control group, respectively. When P>0.05, it indicated that there was no significant difference in PSI levels between the treatment groups and the control group; if P<0.05, it indicated that there were significant differences in PSI levels between the treatment groups and the control group.

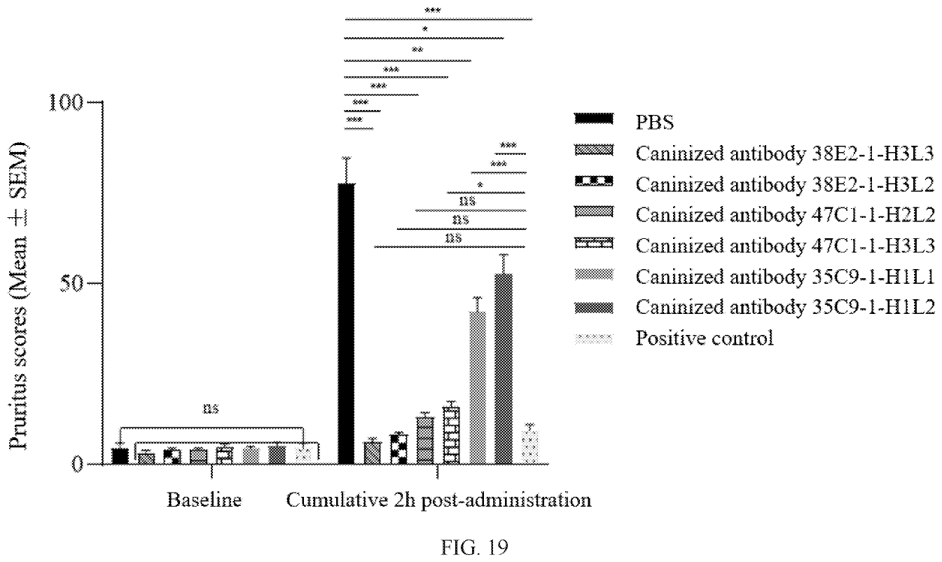

The statistical results of the experiment are shown in FIG. 19. As could be seen from FIG. 19, there were significant differences between the treatment groups and the negative control group. The pharmacodynamic effects of the caninized antibodies 38E2-1-H3L3, 38E2-1-H3L2, and 47C1-1-H2L2 were comparable to those of the positive control; there was a significant difference between the caninized 47C1-1-H3L3 and the positive control, and the caninized antibodies 35C9-1-H1L1 and 35C9-1-H1L2 showed extremely significant differences, with pharmacodynamic effects inferior to that of the positive control.

### 2. Efficacy Study of Caninized Antibody 38E2-1-H3L3

To determine the minimum effective dose and drug duration of the caninized antibody 38E2-1-H3L3, an efficacy study of this antibody was conducted in accordance with the method described in Example 3. The test groups were 0.5 mg/kg, 1 mg/kg, and 2 mg/kg caninized antibody 38E2-1-H3L3, respectively. A commercially available caninized anti-canine IL-31 monoclonal antibody was used as the positive control, and sterile PBS was used as the negative control. 5 experimental canines were selected for each group, requiring a total of 25 experimental canines. Abnormal experimental canines were eliminated via the canine pruritus model. On Day 0 (D0), experimental canines in the treatment groups (5 canines/group) were subcutaneously injected with 0.5 mg/kg, 1 mg/kg, and 2 mg/kg caninized antibody 38E2-1-H3L3 via the cervical region respectively; experimental canines in the positive control group (5 canines/group) were subcutaneously injected with 2 mg/kg caninized anti-canine IL-31 monoclonal antibody via the cervical region; experimental canines in the negative control group (5 canines/group) were subcutaneously injected with an equal volume of sterile PBS via the cervical region. With reference to relevant literatures, 3 μg/kg canine IL-31 was administered to experimental canines via the intravenous route on Day 1 (D1), Day 7 (D7), Day 14 (D14), Day 21 (D21), and Day 28 (D28). Clinical observation was initiated 30 min before each administration and recorded continuously for 30 min; clinical observation was started again 30 min after each administration and recorded continuously for 2 h, with the pruritus frequency of the animals recorded during clinical observation. The 30 min clinical observation before administration on D1 was used to establish a baseline. The PSI results for the cumulative 30 min before injection on D1 and the cumulative 2 h half an hour after injection on D1, D7, D14, D21, and D28 were statistically analyzed; one-way analysis of variance in SPSS software was used to compare the significant differences in PSI between the treatment groups and the control group, respectively. When P>0.05, it indicated that there was no significant difference in PSI levels between the treatment groups and the control group; if P<0.05, it indicated that there were significant differences in PSI levels between the treatment groups and the control group.

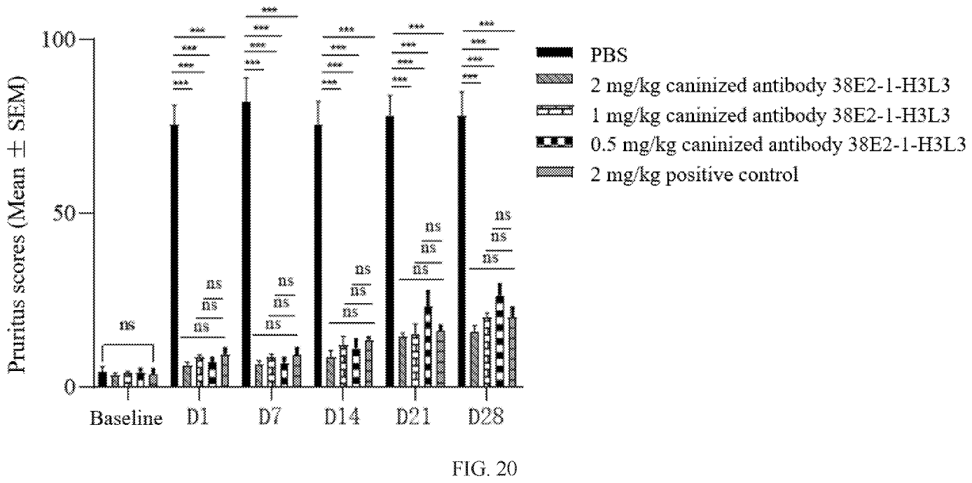

The statistical results of the experiment are shown in FIG. 20. As could be seen from FIG. 20, there were significant differences between the treatment groups and the negative control group. The pharmacodynamic effects of the caninized antibody 38E2-1-H3L3 at doses of 0.5 mg/kg, 1 mg/kg, and 2 mg/kg were comparable to those of the positive control on Day 28 post-administration. These results indicate that the minimum effective dose of the caninized antibody 38E2-1-H3L3 is 0.5 mg/kg, and the drug duration is at least 28 days.

### Example 9: Immunogenicity Evaluation of Caninized Antibody 38E2-1-H3L3

The production of anti-drug antibodies (ADAs) may affect drug activity and half-life. To evaluate the immunogenicity of the caninized antibody 38E2-1-H3L3, with reference to relevant patents and literature, 10 healthy experimental canines were selected and divided into a test group and a control group. Experimental canines in the test group (5 canines/group) were subcutaneously injected with 9 mg/kg caninized antibody 38E2-1-H3L3 via the cervical region on Day 0 (D0), Day 14 (D14), and Day 28 (D28) respectively; experimental canines in the negative control group (5 canines/group) were subcutaneously injected with an equal volume of sterile PBS via the cervical region. Blood samples were collected on DO, D7, D14, D21, and D28 respectively to detect the serum drug concentration and anti-drug antibodies.

### 1. Determination of Blood Drug Concentration

Canine IL-31 at 1 μg/mL was added to a 96-well plate at 100 μL/well and coated overnight at 4° C.; after washing the plate 3 times with 0.1% PBST washing buffer at 300 μL/well, blocking buffer containing 5% skimmed milk powder was added and incubated at 37° C. for 1 h; after washing the plate 3 times, negative serum was diluted 200-fold with PBS containing 1% skimmed milk powder as the diluent, then serially diluted caninized antibody was added and incubated at 37° C. for 1 h; after washing the plate 3 times, Goat-anti-Canine-IgG (H+L)-HRP diluted 1:2000 was added and incubated at 37° C. for 1 h; after washing the plate 3 times, color development was performed with TMB chromogenic solution, the reaction was terminated with 1 M H₂SO₄ solution, and the absorbance was read at 450 nm using a microplate reader.

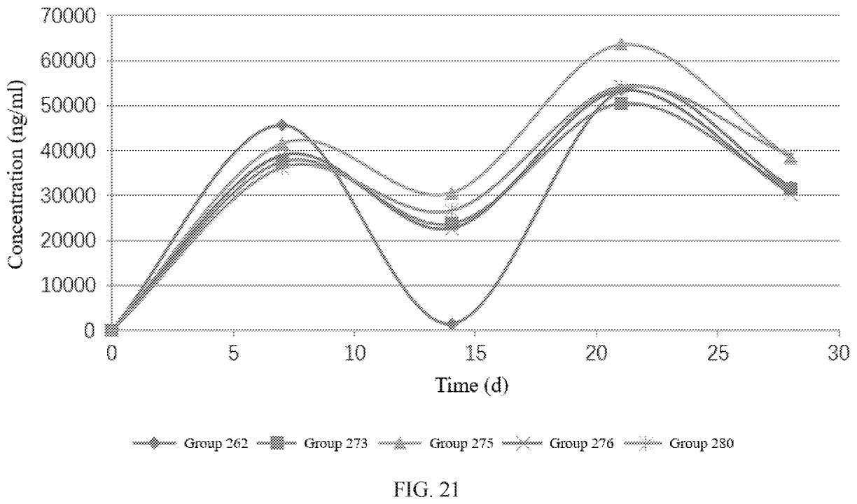

As could be seen from FIG. 21, the drug metabolism profiles in the test group were basically consistent.

### 2. Preparation of ADA-Positive Serum

(1) Caninized antibody 38E2-1-H3L3 was diluted to 1 mg/mL with physiological saline, mixed with an equal volume of adjuvant (Biodragon QuickAntibody-Mouse5W), and then used to immunize 5 mice (Balb/c female mice, 6 weeks old) by intramuscular injection at a dose of 100 μL per mouse.
(2) Three weeks later, caninized antibody 38E2-1-H3L3 was diluted to 1 mg/mL with physiological saline, mixed with an equal volume of adjuvant, and the 5 mice were immunized again by intramuscular injection at 100 μL per mouse.
(3) Blood was collected from each of the 5 mice approximately two weeks later. Serum was obtained by centrifugation at 3000 rpm for 5 min, and the serum titer of the immunized mice was determined by ELISA.

### 3. ELISA Determination of Positive Serum Titer

Caninized antibody 38E2-1-H3L3 at 1 μg/mL was added to a 96-well plate at 100 μL/well and coated overnight at 4° C.; after washing the plate 3 times with 0.1% PBST washing buffer at 300 μL/well, blocking buffer was added and incubated at 37° C. for 1 h; after washing the plate 3 times, the sera from the 5 immunized mice were first diluted 100-fold with blocking buffer, followed by 3-fold serial dilution for 10 points, and added to the 96-well plate at 100 μL/well for incubation at 37° C. for 1 h, with blocking buffer as the blank control; after washing the plate 3 times, Rabbit-anti-mouse-IgG (H+L)-HRP was added and incubated at 37° C. for 1 h; after washing the plate 3 times, color development was performed with TMB chromogenic solution, the reaction was terminated with 1 M H₂SO₄ solution, and the absorbance was read at 450 nm using a microplate reader.

According to the results in Table 19, the average value of the blank control group was approximately 0.45. Using a detection value twice that of 0.45 (i.e., greater than 0.9) as the positive criterion, the serum titers of immunized mice 1-5 were 1:24300, 1:72900, 1:72900, 1:218700, and 1:72900, respectively.

TABLE 19
Determination of Positive Serum Titer

| | 1:100 | 1:300 | 1:900 | 1:2700 | 1:8100 | 1:24300 | 1:72900 | 1:218700 | 1:656100 | 1:1968300 | 1:5904900 | Blank |
|---|---|---|---|---|---|---|---|---|---|---|---|---|
| 1 | 3.475 | 3.249 | 2.92 | 2.508 | 1.844 | 1.179 | 0.778 | 0.581 | 0.536 | 0.503 | 0.48 | 0.479 |
| 2 | 3.44 | 3.402 | 3.259 | 3.183 | 2.787 | 2.055 | 1.359 | 0.889 | 0.628 | 0.519 | 0.52 | 0.436 |
| 3 | 3.318 | 3.026 | 3.089 | 2.817 | 2.464 | 1.792 | 1.134 | 0.75 | 0.579 | 0.494 | 0.464 | 0.428 |
| 4 | 3.293 | 3.087 | 3.061 | 3.009 | 2.806 | 2.316 | 1.696 | 1.105 | 0.722 | 0.579 | 0.498 | 0.427 |
| 5 | 3.348 | 3.329 | 3.248 | 3.024 | 2.518 | 2.033 | 1.306 | 0.843 | 0.628 | 0.505 | 0.493 | 0.439 |

### 3. Determination of Anti-Drug Antibodies

Caninized antibody at 1 μg/mL was added to a 96-well plate at 100 μL/well and coated overnight at 4° C.; after washing the plate 3 times with 0.1% PBST washing buffer at 300 μL/well, blocking buffer containing 5% skimmed milk powder was added and incubated at 37° C. for 1 h; after washing the plate 3 times, mouse polyclonal positive serum containing anti-drug antibodies (serum titer 1:218700) was first 10-fold diluted and then subjected to 2-fold serial dilution for 10 points using PBS containing 1% skimmed milk powder as the dilution buffer; test serum and canine negative serum were both 10-fold diluted with sample diluent and incubated at 37° C. for 1 h; after washing the plate 3 times, biotin-labeled caninized antibody was added; after washing the plate 3 times, streptavidin-HRP was added; after washing the plate 3 times, color development was performed with TMB chromogenic solution, the reaction was terminated with 1 M H₂SO₄ solution, and the absorbance was read at 450 nm using a microplate reader.

Wherein, the results of anti-drug antibody detection for positive serum are shown in Table 20, and the results for the test group and negative control group are shown in Table 21. As could be seen from the above tables, no anti-drug antibodies were detected in any of the test samples, indicating that the immunogenicity risk of caninized antibody 38E2-1-H3L3 is very low.

TABLE 20
Results of Anti-Drug Antibody Assay for Positive Serum

| Titer | 32000 | 16000 | 8000 | 4000 | 2000 | 1000 | 500 | 250 | 125 | 62.5 | 31.25 |
|---|---|---|---|---|---|---|---|---|---|---|---|
| OD₄₅₀ | 3.625 | 3.663 | 3.688 | 3.668 | 3.675 | 3.640 | 3.458 | 3.564 | 1.309 | 0.435 | 0.163 |

TABLE 21
Results of Anti-Drug Antibody Assay for
Test Group and Negative Control Group

| | Experimental | OD₄₅₀ | | | | |
|---|---|---|---|---|---|---|
| Group | canines | D 0 | D 7 | D 14 | D 21 | D 28 |
| Test | 262 | 0.045 | 0.044 | 0.048 | 0.052 | 0.052 |
| Group | 273 | 0.051 | 0.047 | 0.047 | 0.047 | 0.042 |
| | 275 | 0.044 | 0.049 | 0.046 | 0.048 | 0.047 |
| | 276 | 0.050 | 0.053 | 0.054 | 0.050 | 0.044 |
| | 280 | 0.045 | 0.046 | 0.041 | 0.045 | 0.046 |
| Negative | 116 | 0.059 | 0.042 | 0.043 | 0.044 | 0.046 |
| Control | 197 | 0.046 | 0.051 | 0.045 | 0.045 | 0.042 |
| Group | 110 | 0.043 | 0.046 | 0.053 | 0.044 | 0.046 |
| | 126 | 0.049 | 0.046 | 0.047 | 0.046 | 0.049 |
| | 586 | 0.050 | 0.041 | 0.047 | 0.049 | 0.045 |

In summary, the anti-canine IL-31 monoclonal antibodies of the present disclosure can effectively alleviate clinical symptoms associated with canine atopic dermatitis, and the immunogenicity risk of the caninized antibody is very low.

The foregoing descriptions are merely examples of the present disclosure and do not limit the present disclosure in any form or substance. It should be noted that persons of ordinary skill in the art may make several improvements and additions without departing from the method of the present disclosure, and such improvements and additions shall also be regarded as the scope of protection of the present disclosure. Any person skilled in the art, without departing from the spirit and scope of the present disclosure, may make equivalent changes of slight alterations, modifications and evolutions by using the technical contents disclosed above, which are equivalent embodiments of the present disclosure. Meanwhile, any alterations, modifications and evolutions of equivalent changes made to the above embodiments according to the essential technology of the present disclosure shall still fall within the scope of the technical solutions of the present disclosure.

Information of Amino Acid Sequence

| | | Amino Acid Sequence | NO. |
|---|---|---|---|
| Rabbit-derived | HCDR | SYWMS | SEQ ID |
| 35C9-1 | 1 | | NO: 1 |
| antibody | HCDR | DIYATGSTYYANWATG | SEQ ID |
| | 2 | | NO: 11 |
| | HCDR | HSSRSGIDFDL | SEQ ID |
| | 3 | | NO: 21 |
| | VH | QSLEESGGRLVTPGTPLTLTCTASGFSLSSYWMSWVR | SEQ ID |
| | | QAPGKGLEWIGDIYATGSTYYANWATGRFTGSKTST | NO: 61 |
| | | TVDLTITSPTIEDTATYFCARHSSRSGIDFDLWGQGTL | |
| | | VTVSS | |
| | LCDR1 | QASQSIGNYLA | SEQ ID |
| | | | NO: 31 |
| | LCDR2 | RASTLAS | SEQ ID |
| | | | NO: 41 |
| | LCDR3 | QQGYDSNNVDNF | SEQ ID |
| | | | NO: 51 |
| | VL | AYDMTQTPASVEAAVGGTVTIKCQASQSIGNYLAWY | SEQ ID |
| | | QQKPGQPPKQLIYRASTLASGVPSRFKGSGSGTEFTL | NO: 71 |
| | | TISDLECADGATYYCQQGYDSNNVDNFFGGGTEVV | |
| | | VR | |
| Rabbit-derived | HCDR | TYAMN | SEQ ID |
| 47C1-1 | 1 | | NO: 2 |
| antibody | HCDR | IIYSNGDGYYASWAKG | SEQ ID |
| | 2 | | NO: 12 |
| | HCDR | GIYSQTTGYFNL | SEQ ID |
| | 3 | | NO: 22 |
| | VH | QSVEESGGRLVTPGTPLTLTCTVSGFSLSTYAMNWVR | SEQ ID |
| | | QAPGKGLEWIGIIYSNGDGYYASWAKGRYTISKTSST | NO: 62 |
| | | TVDLKVTSPTTEDTATYFCARGIYSQTTGYFNLWGQ | |
| | | GTLVTVSS | |
| | LCDR1 | QASESIGNTLA | SEQ ID |
| | | | NO: 32 |
| | LCDR2 | GASTLAS | SEQ ID |
| | | | NO: 42 |
| | LCDR3 | QSYWSSTTTFRDT | SEQ ID |
| | | | NO: 52 |
| | VL | ADIVMTQTPASVEAAVGGIVIIKCQASESIGNTLAWY | SEQ ID |
| | | QQKPGQPPKLLIYGASTLASGVSSRFKGSGSGTEVTL | NO: 72 |
| | | TISDLECADAATYYCQSYWSSTTTFRDTFGGGTEVV | |
| | | VK | |
| Rabbit-derived | HCDR | GYYMG | SEQ ID |
| 38E2-1 | 1 | | NO: 3 |
| antibody | HCDR | IIGGSSNTFYASWAKG | SEQ ID |
| | 2 | | NO: 13 |
| | HCDR | HGDTDYISGTYFDL | SEQ ID |
| | 3 | | NO: 23 |
| | VH | QSLEESGGRLVTPGTPLTLTCTASAFSLSGYYMGWV | SEQ ID |
| | | RQAPGKGLEYIGIIGGSSNTFYASWAKGRFTISKTPTT | NO: 63 |
| | | VDLKITSPTTEDTATYFCARHGDTDYISGTYFDLWGQ | |
| | | GTLVTVSS | |
| | LCDR1 | QASQSISIYLA | SEQ ID |
| | | | NO: 33 |
| | LCDR2 | RASTLAS | SEQ ID |
| | | | NO: 43 |
| | LCDR3 | QQGYEDSNVESL | SEQ ID |
| | | | NO: 53 |
| | VL | AYHMTQTPASVEVPVGGTVTIKCQASQSISIYLAWY | SEQ ID |
| | | QQKPGQPPKLLIYRASTLASGVPSRFIGSGSGTEFSLT | NO: 73 |
| | | ISDLECADAATYYCQQGYEDSNVESLFGGGTEVVVK | |
| Rabbit-derived | HCDR | SDYYMC | SEQ ID |
| 5H4-2 | 1 | | NO: 4 |
| antibody | HCDR | CITTSTVGTWYASWAKG | SEQ ID |
| | 2 | | NO: 14 |
| | HCDR | EDGTYADYFNL | SEQ ID |
| | 3 | | NO: 24 |
| | VH | QSLEESGGDLVKPGASLTLTCTASGFTLSSDYYMCW | SEQ ID |
| | | VRQAPGKGLELIACITTSTVGTWYASWAKGRFTISKT | NO: 64 |
| | | SSTTVTLQMTSLTAADTATYFCGREDGTYADYFNLW | |
| | | GPGTLVTVSS | |
| | LCDR1 | QASQSISSWLA | SEQ ID |
| | | | NO: 34 |
| | LCDR2 | RASTLAS | SEQ ID |
| | | | NO: 44 |
| | LCDR3 | QQGYRSSNLDNA | SEQ ID |
| | | | NO: 54 |
| | VL | AYDMTQTPASVEVAVGGTVTIKCQASQSISSWLAWY | SEQ ID |
| | | QQKPGQRPKLLIYRASTLASGVSSRFKGSGSGTQFTL | NO: 74 |
| | | TISGVECADAATYYCQQGYRSSNLDNAFGGGTEVV | |
| | | VK | |
| Rabbit-derived | HCDR | SYWMS | SEQ ID |
| 19D1-1 | 1 | | NO: 5 |
| antibody | HCDR | DIYASGSEYYASWVNG | SEQ ID |
| | 2 | | NO: 15 |
| | HCDR | HSSRSGIDFDL | SEQ ID |
| | 3 | | NO: 25 |
| | VH | QSLEESGGRLVTPGTPLTLTCTASGFSLSSYWMSWVR | SEQ ID |
| | | QAPGKGLEWIGDIYASGSEYYASWVNGRFTGSKTST | NO: 65 |
| | | TVDLKITSPTIEDTATYFCARHSSRSGIDFDLWGQGTL | |
| | | VTVSS | |
| | LCDR1 | QASQSISNYLA | SEQ ID |
| | | | NO: 35 |
| | LCDR2 | RASTLAS | SEQ ID |
| | | | NO: 45 |
| | LCDR3 | QQGYDSNNVDNL | SEQ ID |
| | | | NO: 55 |
| | VL | AYDMTQTPASVEAAVGGTVTIKCQASQSISNYLAWY | SEQ ID |
| | | QQKPGQPPKQLIYRASTLASGVPSRFKGSGSGTEFTL | NO: 75 |
| | | TMSDLECADAATYYCQQGYDSNNVDNLFGGGTEV | |
| | | VVK | |
| Rabbit-derived | HCDR | GYYMS | SEQ ID |
| 18E8-1 | 1 | | NO: 6 |
| antibody | HCDR | IIGGSGNTFYASWAKG | SEQ ID |
| | 2 | | NO: 16 |
| | HCDR | HGDTDYISGTYFDL | SEQ ID |
| | 3 | | NO: 26 |
| | VH | QSLEESGGRLVTPGTPLTLTCTASAFSLSGYYMGWV | SEQ ID |
| | | RQAPGKGLEYIGIIGGSSNTFYASWAKGRFTISKTPTT | NO: 66 |
| | | VDLKITSPTTEDTATYFCARHGDTDYISGTYFDLWGQ | |
| | | GTLVTVSS | |
| | LCDR1 | QASQSISIYLA | SEQ ID |
| | | | NO: 36 |
| | LCDR2 | KASTLAS | SEQ ID |
| | | | NO: 46 |
| | LCDR3 | QQGYEDSNVESL | SEQ ID |
| | | | NO: 56 |
| | VL | AYHMTQTPASVEVPVGGTVTIKCQASQSISIYLAWY | SEQ ID |
| | | QQKPGQPPKLLIYKASTLASGVPSRFIGSGSGTEFSLT | NO: 76 |
| | | ISDLECADAATYYCQQGYEDSNVESLFGGGTEVVVK | |
| Rabbit-derived | HCDR | SYAMG | SEQ ID |
| 19C7-1 | 1 | | NO: 7 |
| antibody | HCDR | IINSSGSTWYATWVKG | SEQ ID |
| | 2 | | NO: 17 |
| | HCDR | NRDPFYITDMDL | SEQ ID |
| | 3 | | NO: 27 |
| | VH | QSVEESGGRLVTPGTPLTLTCTVSGFSLNSYAMGWV | SEQ ID |
| | | RQAPGKGLEYIGIINSSGSTWYATWVKGRFTISKTST | |
| | | TVDLKMTSVTTEDTATYFCARNRDPFYITDMDLWG | NO: 67 |
| | | QGTLVTVSS | |
| | LCDR1 | QASQNIYSYLS | SEQ ID |
| | | | NO: 37 |
| | LCDR2 | RASTLAS | SEQ ID |
| | | | NO: 47 |
| | LCDR3 | QQGDSYSDVDNI | SEQ ID |
| | | | NO: 57 |
| | VL | QVLTQTPSSVSAAVGGTVTINCQASQNIYSYLSWYQ | SEQ ID |
| | | QKPGQPPKLLIYRASTLASGVPSRFKGSGSGTEFTLTI | NO: 77 |
| | | SGVECADAATYYCQQGDSYSDVDNIFGGGTEVVVK | |
| Rabbit-derived | HCDR | NSYMG | SEQ ID |
| 44B4-1 | 1 | | NO: 8 |
| antibody | HCDR | FIDTGGTTYYASWAKG | SEQ ID |
| | 2 | | NO: 18 |
| | HCDR | GPDDYFFNL | SEQ ID |
| | 3 | | NO: 28 |
| | VH | QSLEESGGRLVTPGTPLTLTCTASGFSLSNSYMGWVR | SEQ ID |
| | | QAPGKGLEYIGFIDTGGTTYYASWAKGRFTISRTSTT | NO: 68 |
| | | VDLKMTSPTTEDTATYFCAKGPDDYFFNLWGQGTLV | |
| | | TVSS | |
| | LCDR1 | QASESISSWLA | SEQ ID |
| | | | NO: 38 |
| | LCDR2 | KASTLES | SEQ ID |
| | | | NO: 48 |
| | LCDR3 | QVYYFSYNSYNF | SEQ ID |
| | | | NO: 58 |
| | VL | ADIVMTQTPASVEAAVGGTITINCQASESISSWLAWY | SEQ ID |
| | | QQKPGQPPKLLIYKASTLESGVPSRFKGSGSGTEFTL | NO: 78 |
| | | TISGVQCADAATYYCQVYYFSYNSYNFFGGGTEVV | |
| | | VK | |
| Rabbit-derived | HCDR | GYYMN | SEQ ID |
| 30A11-1 | 1 | | NO: 9 |
| antibody | HCDR | FIYSPSTTYYANWAKG | SEQ ID |
| | 2 | | NO: 19 |
| | HCDR | GHTTEIRLDL | SEQ ID |
| | 3 | | NO: 29 |
| | VH | QEQLKESGGGLVTPGGTLTLTCTASGFTISGYYMNW | SEQ ID |
| | | VRQAPGKGLEWIGFIYSPSTTYYANWAKGRFTISKTS | NO: 69 |
| | | TTVDLKITSPTTEDTATYFCARGHTTEIRLDLWGQGT | |
| | | LVTVSS | |
| | LCDR1 | QSSESVYKNNRLS | SEQ ID |
| | | | NO: 39 |
| | LCDR2 | DASTLAS | SEQ ID |
| | | | NO: 49 |
| | LCDR3 | LGEFRDSSDNS | SEQ ID |
| | | | NO: 59 |
| | VL | AVLTQTPSPVSAAVGGTVSISCQSSESVYKNNRLSWF | SEQ ID |
| | | QQKPGQPPKLLIFDASTLASGVSSRFKGSGSGTQFTL | NO: 79 |
| | | TISGVQCDDAATYYCLGEFRDSSDNSFGGGTEVVVK | |
| Rabbit-derived | HCDR | SYAMI | SEQ ID |
| 30G5-1 | 1 | | NO: 10 |
| antibody | HCDR | TIDPSDNTDYASWAKG | SEQ ID |
| | 2 | | NO: 20 |
| | HCDR | HSYDDYGRDVWNL | SEQ ID |
| | 3 | | NO: 30 |
| | VH | QSLEESGGRLVTPGGSLTLTCTVSGFSLSSYAMIWVR | SEQ ID |
| | | QAPGEGLEWIGTIDPSDNTDYASWAKGRFTISKTSST | NO: 70 |
| | | TVDLKLTSLTTEDTATYFCASHSYDDYGRDVWNLW | |
| | | GQGTLVTVSS | |
| | LCDR1 | QASQSVDNNNDLS | SEQ ID |
| | | | NO: 40 |
| | LCDR2 | YTSSLPS | SEQ ID |
| | | | NO: 50 |
| | LCDR3 | VGYKSSTTDATA | SEQ ID |
| | | | NO: 60 |
| | VL | IVMTQTPSSKSVPVGDTVTINCQASQSVDNNNDLSW | SEQ ID |
| | | YQQKPGQPPKLLIYYTSSLPSGVPSRFKGSGSGTQFT | NO: 80 |
| | | LTISDLECDDAATYYCVGYKSSTTDATAFGGGTEVM | |
| | | VK | |
| IgGB | | MESVLFWVFLVTILKGVQGEVRLVESGGTLVKPGGS | SEQ ID |
| | | LKLSCVASGFTFRRYSMDWVRQAPGKSLQWVAGIN | NO: 161 |
| | | GDGTGTSYSQTVKGRFTISRDNAKNTLYLQINSLRAE | |
| | | DSAVYYCAKSWSRNGDLDYWGQGTLVTVSSASTTA | |
| | | PSVFPLAPSCGSTSGSTVALACLVSGYFPEPVTVSWN | |
| | | SGSLTSGVHTFPSVLQSSGLYSLSSMVTVPSSRWPSET | |
| | | FTCNVAHPASKTKVDKPVPKRENGRVPRPPDCPKCP | |
| | | APEMLGGPSVFIFPPKPKDTLLIARTPEVTCVVVDLD | |
| | | PEDPEVQISWFVDGKQMQTAKTQPREEQFNGTYRV | |
| | | VSVLPIGHQDWLKGKQFTCKVNNKALPSPIERTISKA | |
| | | RGQAHQPSVYVLPPSREELSKNTVSLTCLIKDFFPPDI | |
| | | DVEWQSNGQQEPESKYRTTPPQLDEDGSYFLYSKLS | |
| | | VDKSRWQRGDTFICAVMHEALHNHYTQESLSHSPG | |
| | | K | |
| Chimeric | VH | QSLEESGGRLVTPGTPLTLTCTASAFSLSGYYMGWV | SEQ ID |
| Antibody | | RQAPGKGLEYIGIIGGSSNTFYASWAKGRFTISKTPTT | NO: 163 |
| 38E2-1 | | VDLKITSPTTEDTATYFCARHGDTDYISGTYFDLWGQ | |
| | | GTLVTVSSASTTAPSVFPLAPSCGSTSGSTVALACLVS | |
| | | GYFPEPVTVSWNSGSLTSGVHTFPSVLQSSGLYSLSS | |
| | | MVTVPSSRWPSETFTCNVAHPASKTKVDKPVPKREN | |
| | | GRVPRPPDCPKCPAPEMLGGPSVFIFPPKPKDTLLIAR | |
| | | TPEVTCVVVDLDPEDPEVQISWFVDGKQMQTAKTQ | |
| | | PREEQFNGTYRVVSVLPIGHQDWLKGKQFTCKVNN | |
| | | KALPSPIERTISKARGQAHQPSVYVLPPSREELSKNTV | |
| | | SLTCLIKDFFPPDIDVEWQSNGQQEPESKYRTTPPQL | |
| | | DEDGSYFLYSKLSVDKSRWQRGDTFICAVMHEALHN | |
| | | HYTQESLSHSPG | |
| | VL | AYHMTQTPASVEVPVGGTVTIKCQASQSISIYLAWY | SEQ ID |
| | | QQKPGQPPKLLIYRASTLASGVPSRFIGSGSGTEFSLT | NO: 166 |
| | | ISDLECADAATYYCQQGYEDSNVESLFGGGTEVVVK | |
| | | RNDAQPAVYLFQPSPDQLHTGSASVVCLLNSFYPKDI | |
| | | NVKWKVDGVIQDTGIQESVTEQDKDSTYSLSSTLTM | |
| | | SSTEYLSHELYSCEITHKSLPSTLIKSFQRSEC | |
| Chimeric | VH | QSLEESGGRLVTPGTPLTLTCTASGFSLSSYWMSWVR | SEQ ID |
| Antibody | | QAPGKGLEWIGDIYATGSTYYANWATGRFTGSKTST | NO: 164 |
| 35C9-1 | | TVDLTITSPTIEDTATYFCARHSSRSGIDFDLWGQGTL | |
| | | VTVSSASTTAPSVFPLAPSCGSTSGSTVALACLVSGYF | |
| | | PEPVTVSWNSGSLTSGVHTFPSVLQSSGLYSLSSMVT | |
| | | VPSSRWPSETFTCNVAHPASKTKVDKPVPKRENGRV | |
| | | PRPPDCPKCPAPEMLGGPSVFIFPPKPKDTLLIARTPE | |
| | | VTCVVVDLDPEDPEVQISWFVDGKQMQTAKTQPRE | |
| | | EQFNGTYRVVSVLPIGHQDWLKGKQFTCKVNNKAL | |
| | | PSPIERTISKARGQAHQPSVYVLPPSREELSKNTVSLT | |
| | | CLIKDFFPPDIDVEWQSNGQQEPESKYRTTPPQLDED | |
| | | GSYFLYSKLSVDKSRWQRGDTFICAVMHEALHNHYT | |
| | | QESLSHSPG | |
| | VL | AYDMTQTPASVEAAVGGTVTIKCQASQSIGNYLAWY | SEQ ID |
| | | QQKPGQPPKQLIYRASTLASGVPSRFKGSGSGTEFTL | NO: 167 |
| | | TISDLECADGATYYCQQGYDSNNVDNFFGGGTEVV | |
| | | VRRNDAQPAVYLFQPSPDQLHTGSASVVCLLNSFYP | |
| | | KDINVKWKVDGVIQDTGIQESVTEQDKDSTYSLSST | |
| | | LTMSSTEYLSHELYSCEITHKSLPSTLIKSFQRSEC | |
| Chimeric | VH | QSVEESGGRLVTPGTPLTLTCTVSGFSLSTYAMNWVR | SEQ ID |
| Antibody | | QAPGKGLEWIGIIYSNGDGYYASWAKGRYTISKTSST | NO: 165 |
| 47C1-1 | | TVDLKVTSPTTEDTATYFCARGIYSQTTGYFNLWGQ | |
| | | GTLVTVSSASTTAPSVFPLAPSCGSTSGSTVALACLVS | |
| | | GYFPEPVTVSWNSGSLTSGVHTFPSVLQSSGLYSLSS | |
| | | MVTVPSSRWPSETFTCNVAHPASKTKVDKPVPKREN | |
| | | GRVPRPPDCPKCPAPEMLGGPSVFIFPPKPKDTLLIAR | |
| | | TPEVTCVVVDLDPEDPEVQISWFVDGKQMQTAKTQ | |
| | | PREEQFNGTYRVVSVLPIGHQDWLKGKQFTCKVNN | |
| | | KALPSPIERTISKARGQAHQPSVYVLPPSREELSKNTV | |
| | | SLTCLIKDFFPPDIDVEWQSNGQQEPESKYRTTPPQL | |
| | | DEDGSYFLYSKLSVDKSRWQRGDTFICAVMHEALHN | |
| | | HYTQESLSHSPG | |
| | VL | ADIVMTQTPASVEAAVGGIVIIKCQASESIGNTLAWY | SEQ ID |
| | | QQKPGQPPKLLIYGASTLASGVSSRFKGSGSGTEVTL | NO: 168 |
| | | TISDLECADAATYYCQSYWSSTTTFRDTFGGGTEVV | |
| | | VKRNDAQPAVYLFQPSPDQLHTGSASVVCLLNSFYP | |
| | | KDINVKWKVDGVIQDTGIQESVTEQDKDSTYSLSST | |
| | | LTMSSTEYLSHELYSCEITHKSLPSTLIKSFQRSEC | |
| Caninized | VH | EVQLVESGGDLVKPGGSLRLSCVASAFSLSGYYMGW | SEQ ID |
| 38E2-H3L3 | | VRQAPGKGLEWISIIGGSSNTFYASWAKGRFTISKDN | NO: 175 |
| antibody | | SKNTLYLQMNSLRAEDTAVYFCARHGDTDYISGTYF | |
| | | DLWGQGTLVTVSSASTTAPSVFPLAPSCGSTSGSTVA | |
| | | LACLVSGYFPEPVTVSWNSGSLTSGVHTFPSVLQSSG | |
| | | LYSLSSMVTVPSSRWPSETFTCNVAHPASKTKVDKPV | |
| | | PKRENGRVPRPPDCPKCPAPEMLGGPSVFIFPPKPKD | |
| | | TLLIARTPEVTCVVVDLDPEDPEVQISWFVDGKQMQ | |
| | | TAKTQPREEQFNGTYRVVSVLPIGHQDWLKGKQFTC | |
| | | KVNNKALPSPIERTISKARGQAHQPSVYVLPPSREEL | |
| | | SKNTVSLTCLIKDFFPPDIDVEWQSNGQQEPESKYRT | |
| | | TPPQLDEDGSYFLYSKLSVDKSRWQRGDTFICAVMH | |
| | | EALHNHYTQESLSHSPG | |
| | VL | EIVMTQSPASLSLSQGEKVTITCQASQSISIYLAWYQQ | SEQ ID |
| | | KPGQPPKLLIYRASTLASGVPSRFSGSGSGTDFSFTIT | NO: 181 |
| | | SLQPEDVATYYCQQGYEDSNVESLFGQGTKVEIKRN | |
| | | DAQPAVYLFQPSPDQLHTGSASVVCLLNSFYPKDINV | |
| | | KWKVDGVIQDTGIQESVTEQDKDSTYSLSSTLTMSS | |
| | | TEYLSHELYSCEITHKSLPSTLIKSFQRSEC | |
| Caninized | VH | EVQLVESGGDLVKPGGSLRLSCVASAFSLSGYYMGW | SEQ ID |
| 38E2-H3L2 | | VRQAPGKGLEWISIIGGSSNTFYASWAKGRFTISKDN | NO: 176 |
| antibody | | SKNTLYLQMNSLRAEDTAVYFCARHGDTDYISGTYF | |
| | | DLWGQGTLVTVSSASTTAPSVFPLAPSCGSTSGSTVA | |
| | | LACLVSGYFPEPVTVSWNSGSLTSGVHTFPSVLQSSG | |
| | | LYSLSSMVTVPSSRWPSETFTCNVAHPASKTKVDKPV | |
| | | PKRENGRVPRPPDCPKCPAPEMLGGPSVFIFPPKPKD | |
| | | TLLIARTPEVTCVVVDLDPEDPEVQISWFVDGKQMQ | |
| | | TAKTQPREEQFNGTYRVVSVLPIGHQDWLKGKQFTC | |
| | | KVNNKALPSPIERTISKARGQAHQPSVYVLPPSREEL | |
| | | SKNTVSLTCLIKDFFPPDIDVEWQSNGQQEPESKYRT | |
| | | TPPQLDEDGSYFLYSKLSVDKSRWQRGDTFICAVMH | |
| | | EALHNHYTQESLSHSPG | |
| | VL | EIVMTQSPASLSLSQGEKVTITCQASQSISIYLAWYQQ | SEQ ID |
| | | KPGQPPKLLIYRASTLASGVPSRFSGSGSGTDFSFTIS | NO: 182 |
| | | SLQPEDVATYYCQQGYEDSNVESLFGQGTKVEIKRN | |
| | | DAQPAVYLFQPSPDQLHTGSASVVCLLNSFYPKDINV | |
| | | KWKVDGVIQDTGIQESVTEQDKDSTYSLSSTLTMSS | |
| | | TEYLSHELYSCEITHKSLPSTLIKSFQRSEC | |
| Caninized | VH | METGLRWLLLVAVLKGVQCEVQLVESGGDLVKPGG | SEQ ID |
| 47C1-1-H2L2 | | SLRLSCVASGFSLSTYAMNWVRQAPGKGLEWISIIYS | NO: 177 |
| antibody | | NGDGYYASWAKGRFTISRDNAKNTLYLQMNSLRAE | |
| | | DTAVYYCARGIYSQTTGYFNLWGQGTLVTVSSASTT | |
| | | APSVFPLAPSCGSTSGSTVALACLVSGYFPEPVTVSW | |
| | | NSGSLTSGVHTFPSVLQSSGLYSLSSMVTVPSSRWPS | |
| | | ETFTCNVAHPASKTKVDKPVPKRENGRVPRPPDCPK | |
| | | CPAPEMLGGPSVFIFPPKPKDTLLIARTPEVTCVVVDL | |
| | | DPEDPEVQISWFVDGKQMQTAKTQPREEQFNGTYR | |
| | | VVSVLPIGHQDWLKGKQFTCKVNNKALPSPIERTISK | |
| | | ARGQAHQPSVYVLPPSREELSKNTVSLTCLIKDFFPP | |
| | | DIDVEWQSNGQQEPESKYRTTPPQLDEDGSYFLYSK | |
| | | LSVDKSRWQRGDTFICAVMHEALHNHYTQESLSHSP | |
| | | G | |
| | VL | MDTRAPTQLLGLLLLWLPGARCEIVMTQTPASLSLS | SEQ ID |
| | | QEGKVTITCQASESIGNTLAWYQQKPGQPPKLLIYG | NO: 183 |
| | | ASTLASGVPSRFSGSGSGTDFTFTINSLQPEDVATYYC | |
| | | QSYWSSTTTFRDTFGQGTKVEIKRNDAQPAVYLFQP | |
| | | SPDQLHTGSASVVCLLNSFYPKDINVKWKVDGVIQD | |
| | | TGIQESVTEQDKDSTYSLSSTLTMSSTEYLSHELYSCE | |
| | | ITHKSLPSTLIKSFQRSEC | |
| Caninized | VH | METGLRWLLLVAVLKGVQCEVQLVESGGDLVKPGG | SEQ ID |
| 47C1-1-H3L3 | | SLRLSCVASGFSLSTYAMNWVRQAPGKGLEWISIIYS | NO: 178 |
| antibody | | NGDGYYASWAKGRFTISRDNSKNTLYLQMNSLRAE | |
| | | DTAVYYCARGIYSQTTGYFNLWGQGTLVTVSSASTT | |
| | | APSVFPLAPSCGSTSGSTVALACLVSGYFPEPVTVSW | |
| | | NSGSLTSGVHTFPSVLQSSGLYSLSSMVTVPSSRWPS | |
| | | ETFTCNVAHPASKTKVDKPVPKRENGRVPRPPDCPK | |
| | | CPAPEMLGGPSVFIFPPKPKDTLLIARTPEVTCVVVDL | |
| | | DPEDPEVQISWFVDGKQMQTAKTQPREEQFNGTYR | |
| | | VVSVLPIGHQDWLKGKQFTCKVNNKALPSPIERTISK | |
| | | ARGQAHQPSVYVLPPSREELSKNTVSLTCLIKDFFPP | |
| | | DIDVEWQSNGQQEPESKYRTTPPQLDEDGSYFLYSK | |
| | | LSVDKSRWQRGDTFICAVMHEALHNHYTQESLSHSP | |
| | | G | |
| | VL | MDTRAPTQLLGLLLLWLPGARCEIVMTQTPASLSLS | SEQ ID |
| | | QEGKVTITCQASESIGNTLAWYQQKPGQPPKLLIYG | NO: 184 |
| | | ASTLASGVPSRFSGSGSGTDFTFTINSLQPEDVATYYC | |
| | | QSYWSSTTTFRDTFGQGTKVEIKRNDAQPAVYLFQP | |
| | | SPDQLHTGSASVVCLLNSFYPKDINVKWKVDGVIQD | |
| | | TGIQESVTEQDKDSTYSLSSTLTMSSTEYLSHELYSCE | |
| | | ITHKSLPSTLIKSFQRSEC | |
| Caninized | VH | EVQLVESGGDLVKPGGSLRLSCVASGFTFSSYWMSW | SEQ ID |
| 35C9-1-H1L1 | | VRQAPGKGLQWLSDIYATGSTYYANWATGRFTGSRD | NO: 179 |
| antibody | | NAKNTLYLQMNSLTIEDTATYFCARHSSRSGIDFDLW | |
| | | GQGTLVTVSSASTTAPSVFPLAPSCGSTSGSTVALAC | |
| | | LVSGYFPEPVTVSWNSGSLTSGVHTFPSVLQSSGLYS | |
| | | LSSMVTVPSSRWPSETFTCNVAHPASKTKVDKPVPK | |
| | | RENGRVPRPPDCPKCPAPEMLGGPSVFIFPPKPKDTL | |
| | | LIARTPEVTCVVVDLDPEDPEVQISWFVDGKQMQTA | |
| | | KTQPREEQFNGTYRVVSVLPIGHQDWLKGKQFTCK | |
| | | VNNKALPSPIERTISKARGQAHQPSVYVLPPSREELS | |
| | | KNTVSLTCLIKDFFPPDIDVEWQSNGQQEPESKYRTT | |
| | | PPQLDEDGSYFLYSKLSVDKSRWQRGDTFICAVMHE | |
| | | ALHNHYTQESLSHSPG | |
| | VL | EIVMTQSPASLSLSQEEKVTITCQASQSIGNYLAWYQ | SEQ ID |
| | | QKPGQAPKQLIYRASTLASGVPSRFKGSGSGTEFTLT | NO: 185 |
| | | ISDLECADVAVYYCQQGYDSNNVDNFFGQGTKVEIK | |
| | | RNDAQPAVYLFQPSPDQLHTGSASVVCLLNSFYPKDI | |
| | | NVKWKVDGVIQDTGIQESVTEQDKDSTYSLSSTLTM | |
| | | SSTEYLSHELYSCEITHKSLPSTLIKSFQRSEC | |
| Caninized | VH | EVQLVESGGDLVKPGGSLRLSCVASGFTFSSYWMSW | SEQ ID |
| 35C9-1-H1L2 | | VRQAPGKGLQWLSDIYATGSTYYANWATGRFTGSRD | NO: 180 |
| antibody | | NAKNTLYLQMNSLTIEDTATYFCARHSSRSGIDFDLW | |
| | | GQGTLVTVSSASTTAPSVFPLAPSCGSTSGSTVALAC | |
| | | LVSGYFPEPVTVSWNSGSLTSGVHTFPSVLQSSGLYS | |
| | | LSSMVTVPSSRWPSETFTCNVAHPASKTKVDKPVPK | |
| | | RENGRVPRPPDCPKCPAPEMLGGPSVFIFPPKPKDTL | |
| | | LIARTPEVTCVVVDLDPEDPEVQISWFVDGKQMQTA | |
| | | KTQPREEQFNGTYRVVSVLPIGHQDWLKGKQFTCK | |
| | | VNNKALPSPIERTISKARGQAHQPSVYVLPPSREELS | |
| | | KNTVSLTCLIKDFFPPDIDVEWQSNGQQEPESKYRTT | |
| | | PPQLDEDGSYFLYSKLSVDKSRWQRGDTFICAVMHE | |
| | | ALHNHYTQESLSHSPG | |
| | VL | EIVMTQSPASLSLSQEEKVTITCQASQSIGNYLAWYQ | SEQ ID |
| | | QKPGQAPKQLIYRASTLASGVPSRFSGSGSGTDFTLTI | NO: 186 |
| | | SDLECADVAVYYCQQGYDSNNVDNFFGQGTKVEIK | |
| | | RNDAQPAVYLFQPSPDQLHTGSASVVCLLNSFYPKDI | |
| | | NVKWKVDGVIQDTGIQESVTEQDKDSTYSLSSTLTM | |
| | | SSTEYLSHELYSCEITHKSLPSTLIKSFQRSEC | |
| Canine IL-31 | | APTHQLPPSDVRKIILELQPLSRGLLEDYQKKETGVP | SEQ ID |
| | | ESNRTLLLCLTSDSQPPRLNSSAILPYFRAIRPLSDKNI | NO: 199 |
| | | IDKIIEQLDKLKFQHEPETEISVPADTFECKSFILTILQQ | |
| | | FSACLESVFKSLNSGPQHHHHHH* | |

Information of Nucleotide Sequence

| | | Nucleotide sequence | No. |
|---|---|---|---|
| Rabbit-derived | HCDR1 | AGCTACTGGATGAGC | SEQ ID |
| 35C9-1 | | | NO: 81 |
| antibody | HCDR2 | GACATTTATGCTACTGGTAGCACATACTACGCGAACT | SEQ ID |
| | | GGGCGACAGGC | NO: 91 |
| | HCDR3 | CATAGTAGTAGGAGTGGTATTGATTTTGACTTG | SEQ ID |
| | | | NO: |
| | | | 101 |
| | VH | CAGTCGCTGGAGGAGTCCGGGGGTCGCCTGGTCACG | SEQ ID |
| | | CCTGGGACACCCCTGACACTCACCTGCACAGCCTCT | NO: |
| | | GGATTCTCCCTCAGTAGCTACTGGATGAGCTGGGTCC | 141 |
| | | GCCAGGCTCCAGGGAAGGGGCTGGAATGGATCGGAG | |
| | | ACATTTATGCTACTGGTAGCACATACTACGCGAACTG | |
| | | GGCGACAGGCCGATTCACCGGCTCCAAAACCTCGAC | |
| | | CACGGTGGATCTGACAATCACCAGTCCGACAATCGA | |
| | | GGACACGGCCACCTATTTCTGTGCCAGACATAGTAGT | |
| | | AGGAGTGGTATTGATTTTGACTTGTGGGGCCAAGGCA | |
| | | CCCTGGTCACCGTCTCCTCA | |
| | LCDR1 | CAGGCCAGTCAGAGCATTGGTAACTACTTAGCC | SEQ ID |
| | | | NO: |
| | | | 111 |
| | LCDR2 | AGGGCATCCACTCTGGCATCT | SEQ ID |
| | | | NO: |
| | | | 121 |
| | LCDR3 | CAACAGGGTTATGATAGTAATAATGTTGATAATTTT | SEQ ID |
| | | | NO: |
| | | | 131 |
| | VL | GCCTATGATATGACCCAGACTCCAGCCTCCGTGGAGG | SEQ ID |
| | | CAGCTGTGGGAGGCACAGTCACCATCAAGTGCCAGG | NO: |
| | | CCAGTCAGAGCATTGGTAACTACTTAGCCTGGTATCA | 151 |
| | | GCAGAAACCAGGGCAGCCTCCCAAGCAACTGATCTA | |
| | | CAGGGCATCCACTCTGGCATCTGGGGTCCCATCGCGG | |
| | | TTCAAAGGCAGTGGATCTGGGACAGAGTTCACTCTC | |
| | | ACCATCAGCGACCTGGAGTGTGCCGATGGTGCCACTT | |
| | | ACTACTGTCAACAGGGTTATGATAGTAATAATGTTGAT | |
| | | AATTTTTTCGGCGGAGGGACCGAGGTGGTGGTCAGA | |
| Rabbit-derived | HCDR1 | ACCTATGCGATGAAC | SEQ ID |
| 47C1-1 | | | NO: 82 |
| antibody | HCDR2 | ATCATTTATAGTAATGGTGATGGATATTACGCGAGCTG | SEQ ID |
| | | GGCGAAAGGC | NO: 92 |
| | HCDR3 | GGCATTTATTCTCAGACTACTGGCTACTTTAACTTG | SEQ ID |
| | | | NO: |
| | | | 102 |
| | VH | CAGTCGGTGGAGGAGTCCGGGGGTCGCCTGGTCACG | SEQ ID |
| | | CCTGGGACACCCCTGACACTCACCTGCACAGTCTCTG | NO: |
| | | GATTCTCCCTCAGTACCTATGCGATGAACTGGGTCCG | 142 |
| | | CCAGGCTCCAGGGAAGGGGCTGGAATGGATCGGAAT | |
| | | CATTTATAGTAATGGTGATGGATATTACGCGAGCTGGG | |
| | | CGAAAGGCCGATATACCATCTCCAAAACCTCGTCGAC | |
| | | CACGGTGGATCTGAAAGTCACCAGTCCGACAACCGA | |
| | | GGACACGGCCACCTATTTCTGTGCCAGAGGCATTTAT | |
| | | TCTCAGACTACTGGCTACTTTAACTTGTGGGGCCAAG | |
| | | GCACCCTGGTCACCGTCTCATCA | |
| | LCDR1 | CAGGCCAGTGAGAGCATTGGCAATACATTAGCC | SEQ ID |
| | | | NO: |
| | | | 112 |
| | LCDR2 | GGTGCATCCACTCTGGCATCT | SEQ ID |
| | | | NO: |
| | | | 122 |
| | LCDR3 | CAAAGTTATTGGAGTAGTACTACAACTTTCAGGGATA | SEQ ID |
| | | CT | NO: |
| | | | 132 |
| | VL | GCTGACATTGTGATGACCCAGACTCCAGCCTCCGTGG | SEQ ID |
| | | AGGCAGCTGTGGGAGGCATAGTCATCATCAAGTGCC | NO: |
| | | AGGCCAGTGAGAGCATTGGCAATACATTAGCCTGGTA | 152 |
| | | TCAGCAGAAACCAGGGCAGCCTCCCAAGCTCCTGAT | |
| | | TTATGGTGCATCCACTCTGGCATCTGGGGTCTCATCGC | |
| | | GGTTCAAAGGCAGTGGATCTGGGACAGAGGTCACTC | |
| | | TCACCATCAGCGACCTGGAGTGTGCCGATGCTGCCAC | |
| | | TTACTATTGTCAAAGTTATTGGAGTAGTACTACAACTT | |
| | | TCAGGGATACTTTCGGCGGAGGGACCGAGGTGGTGG | |
| | | TCAAA | |
| Rabbit-derived | HCDR1 | GGCTACTACATGGGC | SEQ ID |
| 38E2-1 | | | NO: 83 |
| antibody | HCDR2 | ATCATTGGTGGTAGTAGTAACACATTCTACGCGAGCT | SEQ ID |
| | | GGGCGAAAGGC | NO: 93 |
| | HCDR3 | CATGGGGATACCGATTATATTTCTGGTACCTACTTTGA | SEQ ID |
| | | CTTG | NO: |
| | | | 103 |
| | VH | CAGTCGCTGGAGGAGTCCGGGGGTCGCCTGGTCACG | SEQ ID |
| | | CCTGGGACACCCCTGACACTCACCTGCACAGCCTCT | NO: |
| | | GCATTCTCCCTCAGTGGCTACTACATGGGCTGGGTCC | 143 |
| | | GCCAGGCTCCAGGGAAGGGGCTGGAATACATCGGAA | |
| | | TCATTGGTGGTAGTAGTAACACATTCTACGCGAGCTG | |
| | | GGCGAAAGGCCGATTCACCATCTCCAAAACCCCGAC | |
| | | CACGGTGGATCTGAAAATCACCAGTCCGACAACCGA | |
| | | GGACACGGCCACCTATTTCTGTGCCAGACATGGGGAT | |
| | | ACCGATTATATTTCTGGTACCTACTTTGACTTGTGGGG | |
| | | CCAAGGCACCCTGGTCACCGTCTCCTCA | |
| | LCDR1 | CAGGCCAGTCAGAGCATTAGTATCTACTTAGCC | SEQ ID |
| | | | NO: |
| | | | 113 |
| | LCDR2 | AGGGCATCCACTCTGGCATCT | SEQ ID |
| | | | NO: |
| | | | 123 |
| | LCDR3 | CAACAGGGTTATGAGGATAGTAATGTTGAGAGTCTT | SEQ ID |
| | | | NO: |
| | | | 133 |
| | VL | GCCTATCATATGACCCAGACTCCAGCCTCTGTGGAGG | SEQ ID |
| | | TGCCTGTGGGAGGCACAGTCACCATCAAGTGCCAGG | NO: |
| | | CCAGTCAGAGCATTAGTATCTACTTAGCCTGGTATCAG | 153 |
| | | CAGAAACCAGGGCAGCCTCCCAAGCTCCTGATCTAC | |
| | | AGGGCATCCACTCTGGCATCTGGGGTCCCATCGCGGT | |
| | | TCATAGGCAGTGGATCTGGGACAGAGTTCAGTCTCAC | |
| | | CATCAGCGACCTGGAGTGTGCCGATGCTGCCACTTAC | |
| | | TACTGTCAACAGGGTTATGAGGATAGTAATGTTGAGA | |
| | | GTCTTTTCGGCGGAGGGACCGAGGTGGTGGTCAAA | |
| Rabbit-derived | HCDR1 | AGCGACTACTACATGTGC | SEQ ID |
| 5H4-2 | | | NO: 84 |
| antibody | HCDR2 | TGCATTACTACTAGTACTGTTGGCACATGGTACGCGA | SEQ ID |
| | | GCTGGGCGAAAGGC | NO: 94 |
| | HCDR3 | GAAGATGGTACTTATGCTGATTATTTCAACTTG | SEQ ID |
| | | | NO: |
| | | | 104 |
| | VH | CAGTCGTTGGAGGAGTCCGGGGGAGACCTGGTCAAG | SEQ ID |
| | | CCTGGGGCATCCCTGACACTCACCTGCACAGCCTCTG | NO: |
| | | GATTCACCCTCAGTAGCGACTACTACATGTGCTGGGT | 144 |
| | | CCGCCAGGCTCCAGGGAAGGGGCTGGAATTGATCGC | |
| | | ATGCATTACTACTAGTACTGTTGGCACATGGTACGCGA | |
| | | GCTGGGCGAAAGGCCGATTCACCATCTCCAAAACCT | |
| | | CGTCGACCACGGTGACTCTGCAAATGACCAGTCTGA | |
| | | CAGCCGCGGACACGGCCACCTATTTCTGTGGGAGAG | |
| | | AAGATGGTACTTATGCTGATTATTTCAACTTGTGGGGC | |
| | | CCAGGCACCCTGGTCACCGTCTCCTCA | |
| | LCDR1 | CAGGCCAGTCAGAGCATTAGCAGTTGGTTAGCC | SEQ ID |
| | | | NO: |
| | | | 114 |
| | LCDR2 | AGGGCATCCACTCTGGCATCT | SEQ ID |
| | | | NO: |
| | | | 124 |
| | LCDR3 | CAACAGGGTTATCGTAGTAGTAATTTAGATAATGCT | SEQ ID |
| | | | NO: |
| | | | 134 |
| | VL | GCCTATGATATGACCCAGACTCCAGCCTCTGTGGAGG | SEQ ID |
| | | TAGCTGTGGGAGGCACAGTCACCATCAAGTGCCAGG | NO: |
| | | CCAGTCAGAGCATTAGCAGTTGGTTAGCCTGGTATCA | 154 |
| | | GCAGAAACCAGGGCAGCGTCCCAAGCTCCTGATCTA | |
| | | CAGGGCATCCACTCTGGCATCTGGGGTCTCATCGCGA | |
| | | TTCAAAGGCAGTGGATCTGGGACACAGTTCACTCTC | |
| | | ACCATCAGCGGCGTGGAGTGTGCCGATGCTGCCACTT | |
| | | ACTACTGTCAACAGGGTTATCGTAGTAGTAATTTAGAT | |
| | | AATGCTTTCGGCGGAGGGACCGAGGTGGTGGTCAAA | |
| Rabbit-derived | HCDR1 | AGTTACTGGATGAGC | SEQ ID |
| 19D1-1 | | | NO: 85 |
| antibody | HCDR2 | GACATTTATGCTAGTGGTAGTGAATACTACGCGAGCT | SEQ ID |
| | | GGGTGAATGGC | NO: 95 |
| | HCDR3 | CATAGTAGTAGGAGTGGTATTGATTTTGACTTG | SEQ ID |
| | | | NO: |
| | | | 105 |
| | VH | CAGTCGCTGGAGGAGTCCGGGGGTCGCCTGGTCACG | SEQ ID |
| | | CCTGGGACACCCCTGACACTCACCTGCACAGCCTCT | NO: |
| | | GGATTCTCCCTCAGTAGTTACTGGATGAGCTGGGTCC | 145 |
| | | GCCAGGCTCCAGGGAAGGGGCTGGAATGGATCGGAG | |
| | | ACATTTATGCTAGTGGTAGTGAATACTACGCGAGCTG | |
| | | GGTGAATGGCCGATTCACCGGCTCCAAAACCTCGAC | |
| | | CACGGTGGATCTGAAAATCACCAGTCCGACAATCGA | |
| | | GGACACGGCCACCTATTTCTGTGCCAGACATAGTAGT | |
| | | AGGAGTGGTATTGATTTTGACTTGTGGGGCCAAGGCA | |
| | | CCCTGGTCACCGTCTCCTCA | |
| | LCDR1 | CAGGCCAGTCAGAGCATTAGTAACTACTTAGCC | SEQ ID |
| | | | NO: |
| | | | 115 |
| | LCDR2 | AGGGCATCCACTCTGGCATCT | SEQ ID |
| | | | NO: |
| | | | 125 |
| | LCDR3 | CAACAGGGTTATGATAGTAATAATGTTGATAATCTT | SEQ ID |
| | | | NO: |
| | | | 135 |
| | | GCCTATGATATGACCCAGACTCCAGCCTCCGTGGAGG | SEQ ID |
| | | CAGCTGTGGGAGGCACAGTCACCATCAAGTGCCAGG | |
| | | CCAGTCAGAGCATTAGTAACTACTTAGCCTGGTATCA | |
| | | GCAGAAACCAGGGCAGCCCCCCAAGCAACTGATCTA | |
| | VL | CAGGGCATCCACTCTGGCATCTGGGGTCCCATCGCGG | NO: |
| | | TTCAAAGGCAGTGGATCTGGGACAGAGTTCACTCTC | |
| | | ACCATGAGCGACCTGGAGTGTGCCGATGCTGCCACTT | |
| | | ACTACTGTCAACAGGGTTATGATAGTAATAATGTTGAT | 155 |
| | | AATCTTTTCGGCGGAGGGACCGAGGTGGTGGTCAAA | |
| Rabbit-derived | HCDR1 | GGCTACTACATGAGC | SEQ ID |
| 18E8-1 | | | NO: 86 |
| antibody | HCDR2 | ATCATTGGTGGTAGTGGTAACACATTCTACGCGAGCT | SEQ ID |
| | | GGGCGAAAGGC | NO: 96 |
| | HCDR3 | CATGGGGATACCGATTATATTTCTGGTACCTACTTTGA | SEQ ID |
| | | CTTG | NO: |
| | | | 106 |
| | VH | CAGTCGCTGGAGGAGTCCGGGGGTCGCCTGGTCACG | SEQ ID |
| | | CCTGGGACACCCCTGACACTCACCTGCACAGCCTCT | NO: |
| | | GGGTTCTCCCTCAGTGGCTACTACATGAGCTGGGTCC | 146 |
| | | GCCAGGCTCCAGGGAAGGGGCTGGAATACATCGGAA | |
| | | TCATTGGTGGTAGTGGTAACACATTCTACGCGAGCTG | |
| | | GGCGAAAGGCCGATTCACCATCTCCAAAACCCCGAC | |
| | | CACGGTGGATCTGAAAATCACCAGTCCGACAACCGA | |
| | | GGACACGGCCACCTATTTCTGTGCCAGACATGGGGAT | |
| | | ACCGATTATATTTCTGGTACCTACTTTGACTTGTGGGG | |
| | | CCAAGGCACCCTGGTCACCGTCTCCTCA | |
| | LCDR1 | CAGGCCAGTCAGAGCATTAGTATCTACTTAGCC | SEQ ID |
| | | | NO: |
| | | | 116 |
| | LCDR2 | AAGGCTTCCACTCTGGCATCT | SEQ ID |
| | | | NO |
| | | | 126 |
| | LCDR3 | CAACAGGGTTATGAGGATAGTAATGTTGAGAGTCTT | SEQ ID |
| | | | NO: |
| | | | 136 |
| | VL | GCCTATCATATGACCCAGACTCCAGCCTCTGTGGAGG | SEQ ID |
| | | TGCCTGTGGGAGGCACAGTCACCATCAAGTGCCAGG | NO: |
| | | CCAGTCAGAGCATTAGTATCTACTTAGCCTGGTATCAG | 156 |
| | | CAGAAACCAGGGCAGCCTCCCAAGCTCCTGATCTAC | |
| | | AAGGCTTCCACTCTGGCATCTGGGGTCCCATCGCGGT | |
| | | TCATAGGCAGTGGATCTGGGACAGAGTTCAGTCTCAC | |
| | | CATCAGCGACCTGGAGTGTGCCGATGCTGCCACTTAC | |
| | | TACTGTCAACAGGGTTATGAGGATAGTAATGTTGAGA | |
| | | GTCTTTTCGGCGGAGGGACCGAGGTGGTGGTCAAA | |
| Rabbit-derived | HCDR1 | AGCTATGCAATGGGC | SEQ ID |
| 19C7-1 | | | NO: 87 |
| antibody | HCDR2 | ATCATTAATAGTAGTGGTAGCACATGGTACGCGACCT | SEQ ID |
| | | GGGTGAAAGGC | NO: 97 |
| | HCDR3 | AATAGAGATCCTTTTTACATTACTGATATGGACTTG | SEQ ID |
| | | | NO: |
| | | | 107 |
| | VH | CAGTCGGTGGAGGAGTCCGGGGGTCGCCTGGTCACG | SEQ ID |
| | | CCTGGGACACCCCTGACACTCACCTGCACAGTCTCTG | NO: |
| | | GATTCTCCCTCAATAGCTATGCAATGGGCTGGGTCCG | 147 |
| | | CCAGGCTCCAGGGAAGGGGCTGGAATACATCGGAAT | |
| | | CATTAATAGTAGTGGTAGCACATGGTACGCGACCTGG | |
| | | GTGAAAGGCCGATTCACCATCTCCAAAACCTCGACC | |
| | | ACGGTGGATCTGAAAATGACCAGTGTGACAACCGAG | |
| | | GACACGGCCACCTATTTCTGTGCCAGAAATAGAGATC | |
| | | CTTTTTACATTACTGATATGGACTTGTGGGGCCAAGGC | |
| | | ACCCTGGTCACCGTCTCCTCA | |
| | LCDR1 | CAGGCCAGTCAGAACATTTACAGCTACTTATCC | SEQ ID |
| | | | NO: |
| | | | 117 |
| | LCDR2 | AGGGCATCCACTCTGGCATCT | SEQ ID |
| | | | NO: |
| | | | 127 |
| | LCDR3 | CAACAGGGTGATAGTTATAGTGATGTTGATAATATT | SEQ ID |
| | | | NO: |
| | | | 137 |
| | VL | CAAGTGCTGACCCAGACTCCATCCTCCGTGTCTGCAG | SEQ ID |
| | | CTGTGGGAGGCACAGTCACCATCAATTGCCAGGCCA | NO: |
| | | GTCAGAACATTTACAGCTACTTATCCTGGTATCAGCAG | 157 |
| | | AAACCAGGGCAGCCTCCCAAGCTCCTGATCTACAGG | |
| | | GCATCCACTCTGGCATCTGGGGTCCCATCGCGGTTCA | |
| | | AAGGCAGTGGATCTGGGACAGAGTTCACTCTCACCA | |
| | | TCAGCGGCGTGGAGTGTGCCGATGCTGCCACTTACTA | |
| | | CTGTCAACAGGGTGATAGTTATAGTGATGTTGATAATA | |
| | | TTTTCGGCGGAGGGACCGAGGTGGTGGTCAAA | |
| Rabbit-derived | HCDR1 | AACTCGTACATGGGC | SEQ ID |
| 44B4-1 | | | NO: 88 |
| antibody | HCDR2 | TTTATTGACACTGGTGGTACCACATACTACGCGAGCT | SEQ ID |
| | | GGGCGAAAGGC | NO: 98 |
| | HCDR3 | GGGCCTGATGATTATTTCTTTAATTTG | SEQ ID |
| | | | NO: |
| | | | 108 |
| | VH | CAGTCGCTGGAGGAGTCCGGGGGTCGCCTGGTCACG | SEQ ID |
| | | CCTGGGACACCCCTGACACTCACCTGCACAGCCTCT | NO: |
| | | GGATTCTCCCTCAGTAACTCGTACATGGGCTGGGTCC | 148 |
| | | GCCAGGCTCCAGGGAAGGGGCTGGAATACATCGGAT | |
| | | TTATTGACACTGGTGGTACCACATACTACGCGAGCTG | |
| | | GGCGAAAGGCCGATTCACCATCTCCAGAACCTCGAC | |
| | | CACGGTGGATCTGAAAATGACCAGTCCGACAACCGA | |
| | | GGACACGGCCACCTATTTCTGTGCCAAAGGGCCTGAT | |
| | | GATTATTTCTTTAATTTGTGGGGCCAAGGCACCCTGGT | |
| | | CACCGTCTCCTCA | |
| | LCDR1 | CAAGCCAGTGAGAGCATTAGCAGTTGGTTAGCC | SEQ ID |
| | | | NO: |
| | | | 118 |
| | LCDR2 | AAGGCATCCACTCTGGAATCT | SEQ ID |
| | | | NO: |
| | | | 128 |
| | LCDR3 | CAGGTCTATTATTTTAGTTATAATAGTTACAATTTT | SEQ ID |
| | | | NO: |
| | | | 138 |
| | VL | GCTGACATTGTGATGACCCAGACTCCAGCCTCCGTGG | SEQ ID |
| | | AGGCAGCTGTGGGAGGCACAATCACCATCAATTGCC | NO: |
| | | AAGCCAGTGAGAGCATTAGCAGTTGGTTAGCCTGGTA | 158 |
| | | TCAGCAGAAACCAGGGCAGCCTCCCAAGCTCCTGAT | |
| | | CTACAAGGCATCCACTCTGGAATCTGGGGTCCCATCG | |
| | | CGGTTCAAAGGCAGTGGATCTGGGACAGAGTTCACT | |
| | | CTCACCATCAGCGGCGTGCAGTGTGCCGATGCTGCCA | |
| | | CTTACTACTGTCAGGTCTATTATTTTAGTTATAATAGTT | |
| | | ACAATTTTTTCGGCGGAGGGACCGAGGTGGTGGTCA | |
| | | AG | |
| Rabbit-derived | HCDR1 | GGCTACTACATGAAC | SEQ ID |
| 30A11-1 | | | NO: 89 |
| antibody | HCDR2 | TTCATTTATAGTCCTAGTACCACATACTACGCGAACTG | SEQ ID |
| | | GGCGAAAGGC | NO: 99 |
| | HCDR3 | GGTCATACTACTGAAATTCGGTTGGATCTC | SEQ ID |
| | | | NO: |
| | | | 109 |
| | VH | CAGGAGCAGCTGAAGGAGTCCGGAGGAGGCCTGGT | SEQ ID |
| | | AACGCCTGGAGGAACCCTGACACTCACCTGCACAGC | NO: |
| | | CTCTGGATTCACCATCAGTGGCTACTACATGAACTGG | 149 |
| | | GTCCGCCAGGCTCCAGGGAAGGGGCTGGAATGGATC | |
| | | GGATTCATTTATAGTCCTAGTACCACATACTACGCGAA | |
| | | CTGGGCGAAAGGCCGATTCACCATCTCCAAAACCTC | |
| | | GACCACCGTGGATCTGAAAATCACCAGTCCGACAAC | |
| | | CGAGGACACGGCCACCTATTTTTGTGCCAGAGGTCAT | |
| | | ACTACTGAAATTCGGTTGGATCTCTGGGGCCAGGGCA | |
| | | CCCTGGTCACCGTCTCCTCA | |
| | LCDR1 | CAGTCCAGTGAGAGTGTTTATAAGAACAACCGCTTAT | SEQ ID |
| | | CC | NO: |
| | | | 119 |
| | LCDR2 | GATGCATCCACTCTGGCATCT | SEQ ID |
| | | | NO: |
| | | | 129 |
| | LCDR3 | CTAGGCGAATTTAGGGATAGTAGTGATAATAGT | SEQ ID |
| | | | NO: |
| | | | 139 |
| | VL | GCCGTGCTGACCCAGACTCCATCTCCCGTGTCTGCAG | SEQ ID |
| | | CTGTGGGAGGCACAGTCAGCATCAGTTGCCAGTCCA | NO: |
| | | GTGAGAGTGTTTATAAGAACAACCGCTTATCCTGGTT | 159 |
| | | TCAGCAGAAACCAGGGCAGCCTCCCAAACTCCTGAT | |
| | | CTTTGATGCATCCACTCTGGCATCTGGGGTCTCATCGC | |
| | | GATTCAAAGGCAGTGGATCTGGGACACAGTTCACTCT | |
| | | CACCATCAGTGGCGTGCAGTGTGACGATGCTGCCACT | |
| | | TACTACTGTCTAGGCGAATTTAGGGATAGTAGTGATAA | |
| | | TAGTTTCGGCGGAGGGACCGAGGTGGTGGTCAAA | |
| Rabbit-derived | HCDR1 | AGCTATGCAATGATC | SEQ ID |
| 30G5-1 | | | NO: 90 |
| antibody | HCDR2 | ACCATTGATCCTAGTGATAACACCGACTACGCGAGTT | SEQ ID |
| | | GGGCAAAAGGC | NO: |
| | | | 100 |
| | HCDR3 | CATTCCTACGATGACTATGGTAGGGACGTCTGGAACT | SEQ ID |
| | | TG | NO: |
| | | | 110 |
| | VH | CAGTCGCTGGAGGAGTCCGGGGGTCGCCTGGTAACG | SEQ ID |
| | | CCTGGAGGATCCCTGACACTCACCTGCACAGTCTCTG | NO: |
| | | GATTCTCCCTCAGTAGCTATGCAATGATCTGGGTCCGC | 150 |
| | | CAGGCTCCAGGGGAGGGGCTGGAATGGATCGGAACC | |
| | | ATTGATCCTAGTGATAACACCGACTACGCGAGTTGGG | |
| | | CAAAAGGCCGATTCACCATCTCCAAAACCTCGTCGAC | |
| | | CACGGTGGATCTGAAATTGACCAGTCTGACAACCGA | |
| | | GGACACGGCCACCTATTTCTGTGCCAGTCATTCCTAC | |
| | | GATGACTATGGTAGGGACGTCTGGAACTTGTGGGGCC | |
| | | AAGGCACCCTGGTCACCGTCTCCTCA | |
| | LCDR1 | CAGGCCAGTCAGAGTGTTGATAATAACAACGACTTAT | SEQ ID |
| | | CC | NO: |
| | | | 120 |
| | LCDR2 | TATACATCCAGCCTGCCATCT | SEQ ID |
| | | | NO: |
| | | | 130 |
| | LCDR3 | GTAGGGTATAAAAGTAGTACTACTGATGCTACTGCT | SEQ ID |
| | | | NO: |
| | | | 140 |
| | VL | ATCGTGATGACCCAGACTCCATCTTCCAAGTCTGTCC | SEQ ID |
| | | CTGTGGGAGACACAGTCACCATCAATTGCCAGGCCA | NO: |
| | | GTCAGAGTGTTGATAATAACAACGACTTATCCTGGTAT | 160 |
| | | CAGCAGAAACCAGGGCAGCCTCCCAAGCTCCTGATC | |
| | | TATTATACATCCAGCCTGCCATCTGGGGTCCCATCGCG | |
| | | GTTCAAAGGCAGTGGATCTGGGACACAGTTCACTCT | |
| | | CACCATCAGCGACCTGGAGTGTGACGATGCTGCCACT | |
| | | TACTACTGTGTAGGGTATAAAAGTAGTACTACTGATGC | |
| | | TACTGCTTTCGGCGGAGGGACCGAGGTGATGGTCAA | |
| | | A | |
| IgGB | | AGTGCTCAGGACACCACACAGACAAATCACCATGGA | SEQ ID |
| | | GTCTGTGCTCTTCTGGGTTTTCCTTGTCACTATTTTAA | NO: |
| | | AAGGTGTCCAGGGTGAGGTACGTTTGGTGGAGTCTG | 162 |
| | | GAGGAACCCTGGTGAAGCCTGGGGGGTCCCTGAAAC | |
| | | TCTCTTGTGTGGCCTCTGGATTCACCTTCAGAAGATA | |
| | | CTCCATGGACTGGGTCCGCCAGGCTCCAGGCAAGAG | |
| | | CCTGCAGTGGGTCGCCGGGATTAACGGTGATGGCAC | |
| | | AGGAACATCCTATTCACAGACTGTGAAGGGCCGATTC | |
| | | ACCATCTCCAGAGACAACGCCAAGAACACCCTCTATC | |
| | | TGCAGATAAACAGCCTGAGAGCCGAAGACTCTGCTG | |
| | | TGTATTATTGTGCCAAGAGCTGGTCTCGTAATGGGGAT | |
| | | CTTGACTACTGGGGCCAGGGAACCCTGGTCACCGTC | |
| | | TCCTCAGCCTCCACCACGGCCCCCTCGGTTTTCCCAC | |
| | | TGGCCCCCAGCTGCGGGTCCACTTCCGGCTCCACGGT | |
| | | GGCCCTGGCCTGCCTGGTGTCAGGCTACTTCCCCGAG | |
| | | CCTGTAACTGTGTCCTGGAATTCCGGCTCCTTGACCA | |
| | | GCGGTGTGCACACCTTCCCGTCCGTCCTGCAGTCCTC | |
| | | AGGGCTCTACTCCCTCAGCAGCATGGTGACAGTGCCC | |
| | | TCCAGCAGGTGGCCCAGCGAGACCTTCACCTGCAAC | |
| | | GTGGCCCACCCGGCCAGCAAAACTAAAGTAGACAAG | |
| | | CCAGTGCCCAAAAGAGAAAATGGAAGAGTTCCTCGC | |
| | | CCACCTGATTGTCCCAAATGCCCAGCCCCTGAAATGC | |
| | | TGGGAGGGCCTTCGGTCTTCATCTTTCCCCCGAAACC | |
| | | CAAGGACACCCTCTTGATTGCCCGAACACCTGAGGT | |
| | | CACATGTGTGGTGGTGGATCTGGACCCAGAAGACCC | |
| | | TGAGGTGCAGATCAGCTGGTTCGTGGACGGTAAGCA | |
| | | GATGCAAACAGCCAAGACTCAGCCTCGTGAGGAGCA | |
| | | GTTCAATGGCACCTACCGTGTGGTCAGTGTCCTCCCC | |
| | | ATTGGGCACCAGGACTGGCTCAAGGGGAAGCAGTTC | |
| | | ACGTGCAAAGTCAACAACAAAGCCCTCCCATCCCCG | |
| | | ATCGAGAGGACCATCTCCAAGGCCAGAGGGCAAGCC | |
| | | CATCAGCCCAGTGTGTATGTCCTGCCGCCATCCCGGG | |
| | | AGGAGTTGAGCAAGAACACAGTCAGCTTGACATGCC | |
| | | TGATCAAAGACTTCTTCCCACCTGACATTGATGTGGA | |
| | | GTGGCAGAGCAATGGACAGCAGGAGCCTGAGAGCA | |
| | | AGTACCGCACGACCCCGCCCCAGCTGGACGAGGACG | |
| | | GGTCCTACTTCCTGTACAGCAAGCTCTCTGTGGACAA | |
| | | GAGCCGCTGGCAGCGGGGAGACACCTTCATATGTGC | |
| | | GGTGATGCATGAAGCTCTACACAACCACTACACACAG | |
| | | GAATCCCTCTCCCATTCTCCGGGTAAATGA | |
| Chimeric | VH | CAAAGTCTAGAAGAAAGTGGTGGTCGACTAGTCACA | SEQ ID |
| Antibody | | CCCGGAACCCCTCTGACACTGACCTGTACCGCCTCTG | NO: |
| 38E2-1 | | GCTTTTCTCTGTCCTCCTACTGGATGTCCTGGGTGCG | 169 |
| | | GCAGGCTCCTGGCAAGGGCCTGGAATGGATCGGCGA | |
| | | CATCTACGCTACCGGCTCCACCTACTACGCCAACTGG | |
| | | GCCACCGGCAGATTCACCGGATCCAAGACCTCTACCA | |
| | | CCGTGGATCTCACAATCACCTCTCCTACCATCGAGGA | |
| | | CACCGCTACATATTTCTGCGCCAGACACTCTAGCCGG | |
| | | TCCGGCATCGACTTCGACCTGTGGGGCCAGGGCACC | |
| | | CTGGTGACCGTGTCCAGCGCTTCCACCACAGCTCCTT | |
| | | CTGTGTTCCCTCTGGCTCCCTCCTGCGGCTCTACCTCC | |
| | | GGATCCACAGTGGCTCTGGCCTGCCTGGTTTCCGGCT | |
| | | ACTTTCCCGAACCTGTCACCGTCTCTTGGAACTCCGG | |
| | | CTCCCTGACCTCTGGCGTGCACACCTTCCCTAGCGTG | |
| | | CTGCAGAGCTCTGGCCTGTACTCCCTGTCCTCCATGG | |
| | | TGACCGTGCCAAGCTCTCGGTGGCCTTCCGAGACATT | |
| | | TACATGCAACGTGGCCCACCCTGCTTCGAAAACAAA | |
| | | AGTGGACAAGCCTGTGCCAAAACGGGAGAATGGCAG | |
| | | AGTGCCTCGGCCTCCCGACTGTCCTAAGTGCCCTGCC | |
| | | CCTGAGATGCTGGGCGGGCCTTCTGTGTTCATCTTCC | |
| | | CACCTAAGCCCAAGGATACCCTGCTGATCGCCAGAAC | |
| | | TCCTGAGGTGACCTGTGTGGTGGTCGACCTGGATCCT | |
| | | GAGGACCCCGAAGTGCAGATCTCTTGGTTCGTGGAC | |
| | | GGCAAACAGATGCAGACCGCCAAGACCCAGCCTAGA | |
| | | GAGGAACAGTTCAACGGCACCTACCGCGTCGTGTCC | |
| | | GTGCTGCCTATCGGCCACCAGGACTGGCTGAAGGGC | |
| | | AAGCAGTTTACCTGCAAGGTGAACAACAAGGCTCTG | |
| | | CCTTCTCCCATCGAGCGGACCATCTCCAAGGCCCGGG | |
| | | GACAGGCCCATCAACCCAGCGTGTACGTGTTGCCCCC | |
| | | AAGCAGAGAAGAGCTCTCTAAGAACACCGTGAGCCT | |
| | | GACCTGTCTGATCAAGGACTTCTTCCCTCCTGACATC | |
| | | GACGTGGAATGGCAGTCCAACGGCCAGCAGGAACCT | |
| | | GAGTCCAAGTACAGAACCACCCCTCCTCAGCTGGAC | |
| | | GAGGATGGCTCCTACTTCCTGTATTCCAAGCTGTCCG | |
| | | TGGATAAGTCCAGATGGCAGAGAGGCGACACCTTCA | |
| | | TTTGCGCCGTGATGCACGAGGCCCTGCACAACCACTA | |
| | | CACCCAAGAGTCTCTGTCTCACTCTCCTGGCTGA | |
| | VL | GCATATGATATGACTCAAACTCCAGCAAGTGTCGAGG | SEQ ID |
| | | CCGCCGTGGGCGGCACCGTGACCATCAAGTGCCAGG | NO: |
| | | CCTCTCAGTCCATCGGCAACTACCTGGCCTGGTATCA | 172 |
| | | GCAGAAGCCTGGCCAGCCTCCTAAGCAGCTGATCTAC | |
| | | AGAGCCAGCACACTGGCTTCTGGCGTGCCCAGCAGA | |
| | | TTTAAAGGCTCTGGCTCCGGCACAGAGTTCACCCTGA | |
| | | CCATCTCCGACCTGGAATGTGCTGATGGCGCTACCTA | |
| | | CTACTGCCAACAGGGCTACGACTCCAACAATGTGGA | |
| | | CAACTTCTTCGGCGGAGGAACCGAGGTGGTGGTGCG | |
| | | GCGGAACGACGCCCAGCCTGCCGTGTACCTGTTCCA | |
| | | GCCCTCTCCAGATCAGCTGCACACAGGATCTGCTTCT | |
| | | GTGGTGTGCCTGCTGAACTCCTTCTACCCTAAAGACA | |
| | | TCAACGTGAAGTGGAAGGTGGACGGCGTGATCCAGG | |
| | | ATACCGGCATCCAAGAGTCCGTGACCGAACAGGACA | |
| | | AGGACTCCACCTACTCTCTGTCCAGCACACTGACCAT | |
| | | GTCCAGCACCGAGTATCTCAGCCATGAACTGTACTCC | |
| | | TGCGAGATCACCCACAAGTCCCTGCCTTCTACCCTGA | |
| | | TCAAGTCTTTTCAGAGATCCGAGTGTTGA | |
| Chimeric | VH | CAAAGTCTAGAAGAAAGTGGTGGTCGACTAGTCACA | SEQ ID |
| Antibody | | CCCGGAACCCCTCTGACACTGACCTGTACCGCCTCTG | NO: |
| 35C9-1 | | GCTTTTCTCTGTCCTCCTACTGGATGTCCTGGGTGCG | 170 |
| | | GCAGGCTCCTGGCAAGGGCCTGGAATGGATCGGCGA | |
| | | CATCTACGCTACCGGCTCCACCTACTACGCCAACTGG | |
| | | GCCACCGGCAGATTCACCGGATCCAAGACCTCTACCA | |
| | | CCGTGGATCTCACAATCACCTCTCCTACCATCGAGGA | |
| | | CACCGCTACATATTTCTGCGCCAGACACTCTAGCCGG | |
| | | TCCGGCATCGACTTCGACCTGTGGGGCCAGGGCACC | |
| | | CTGGTGACCGTGTCCAGCGCTTCCACCACAGCTCCTT | |
| | | CTGTGTTCCCTCTGGCTCCCTCCTGCGGCTCTACCTCC | |
| | | GGATCCACAGTGGCTCTGGCCTGCCTGGTTTCCGGCT | |
| | | ACTTTCCCGAACCTGTCACCGTCTCTTGGAACTCCGG | |
| | | CTCCCTGACCTCTGGCGTGCACACCTTCCCTAGCGTG | |
| | | CTGCAGAGCTCTGGCCTGTACTCCCTGTCCTCCATGG | |
| | | TGACCGTGCCAAGCTCTCGGTGGCCTTCCGAGACATT | |
| | | TACATGCAACGTGGCCCACCCTGCTTCGAAAACAAA | |
| | | AGTGGACAAGCCTGTGCCAAAACGGGAGAATGGCAG | |
| | | AGTGCCTCGGCCTCCCGACTGTCCTAAGTGCCCTGCC | |
| | | CCTGAGATGCTGGGCGGGCCTTCTGTGTTCATCTTCC | |
| | | CACCTAAGCCCAAGGATACCCTGCTGATCGCCAGAAC | |
| | | TCCTGAGGTGACCTGTGTGGTGGTCGACCTGGATCCT | |
| | | GAGGACCCCGAAGTGCAGATCTCTTGGTTCGTGGAC | |
| | | GGCAAACAGATGCAGACCGCCAAGACCCAGCCTAGA | |
| | | GAGGAACAGTTCAACGGCACCTACCGCGTCGTGTCC | |
| | | GTGCTGCCTATCGGCCACCAGGACTGGCTGAAGGGC | |
| | | AAGCAGTTTACCTGCAAGGTGAACAACAAGGCTCTG | |
| | | CCTTCTCCCATCGAGCGGACCATCTCCAAGGCCCGGG | |
| | | GACAGGCCCATCAACCCAGCGTGTACGTGTTGCCCCC | |
| | | AAGCAGAGAAGAGCTCTCTAAGAACACCGTGAGCCT | |
| | | GACCTGTCTGATCAAGGACTTCTTCCCTCCTGACATC | |
| | | GACGTGGAATGGCAGTCCAACGGCCAGCAGGAACCT | |
| | | GAGTCCAAGTACAGAACCACCCCTCCTCAGCTGGAC | |
| | | GAGGATGGCTCCTACTTCCTGTATTCCAAGCTGTCCG | |
| | | TGGATAAGTCCAGATGGCAGAGAGGCGACACCTTCA | |
| | | TTTGCGCCGTGATGCACGAGGCCCTGCACAACCACTA | |
| | | CACCCAAGAGTCTCTGTCTCACTCTCCTGGCTGA | |
| | VL | GCATATGATATGACTCAAACTCCAGCAAGTGTCGAGG | SEQ ID |
| | | CCGCCGTGGGCGGCACCGTGACCATCAAGTGCCAGG | NO: |
| | | CCTCTCAGTCCATCGGCAACTACCTGGCCTGGTATCA | 173 |
| | | GCAGAAGCCTGGCCAGCCTCCTAAGCAGCTGATCTAC | |
| | | AGAGCCAGCACACTGGCTTCTGGCGTGCCCAGCAGA | |
| | | TTTAAAGGCTCTGGCTCCGGCACAGAGTTCACCCTGA | |
| | | CCATCTCCGACCTGGAATGTGCTGATGGCGCTACCTA | |
| | | CTACTGCCAACAGGGCTACGACTCCAACAATGTGGA | |
| | | CAACTTCTTCGGCGGAGGAACCGAGGTGGTGGTGCG | |
| | | GCGGAACGACGCCCAGCCTGCCGTGTACCTGTTCCA | |
| | | GCCCTCTCCAGATCAGCTGCACACAGGATCTGCTTCT | |
| | | GTGGTGTGCCTGCTGAACTCCTTCTACCCTAAAGACA | |
| | | TCAACGTGAAGTGGAAGGTGGACGGCGTGATCCAGG | |
| | | ATACCGGCATCCAAGAGTCCGTGACCGAACAGGACA | |
| | | AGGACTCCACCTACTCTCTGTCCAGCACACTGACCAT | |
| | | GTCCAGCACCGAGTATCTCAGCCATGAACTGTACTCC | |
| | | TGCGAGATCACCCACAAGTCCCTGCCTTCTACCCTGA | |
| | | TCAAGTCTTTTCAGAGATCCGAGTGTTGA | |
| Chimeric | VH | CAGTCTGTGGAAGAATCCGGCGGCAGACTGGTGACA | SEQ ID |
| Antibody | | CCTGGCACCCCTCTGACCCTAACCTGCACAGTGTCTG | NO: |
| 47C1-1 | | GCTTCAGCCTCAGCACCTATGCCATGAACTGGGTGCG | 171 |
| | | GCAAGCTCCTGGCAAGGGCCTGGAATGGATCGGCAT | |
| | | CATCTACTCCAACGGCGACGGCTACTACGCCTCCTGG | |
| | | GCCAAGGGCAGATACACAATTTCCAAAACCAGCTCC | |
| | | ACAACCGTGGATCTGAAGGTGACCTCTCCTACCACCG | |
| | | AAGATACCGCTACCTACTTCTGTGCTAGAGGCATCTA | |
| | | CAGTCAGACCACCGGCTATTTCAACCTGTGGGGCCA | |
| | | AGGCACCCTGGTCACCGTGTCTTCCGCCTCCACCACC | |
| | | GCCCCTTCCGTGTTTCCTCTGGCTCCTAGCTGCGGCT | |
| | | CTACCTCCGGCTCTACCGTGGCTCTGGCCTGCCTGGT | |
| | | ATCTGGCTACTTCCCTGAGCCTGTGACCGTGTCTTGG | |
| | | AACTCCGGAAGCCTGACCTCAGGAGTGCACACCTTT | |
| | | CCATCTGTCCTGCAGTCCTCTGGCCTGTACTCTCTGA | |
| | | GCTCCATGGTGACTGTGCCTTCATCCAGATGGCCTTC | |
| | | CGAGACCTTCACATGCAACGTGGCCCATCCTGCTTCC | |
| | | AAGACCAAGGTCGACAAGCCTGTGCCAAAGAGAGA | |
| | | GAACGGAAGAGTGCCCCGGCCTCCTGACTGCCCCAA | |
| | | GTGCCCTGCTCCAGAGATGCTGGGCGGCCCTAGCGT | |
| | | GTTCATCTTCCCACCTAAGCCCAAGGACACCCTGCTG | |
| | | ATCGCCAGAACCCCTGAAGTGACATGTGTGGTGGTG | |
| | | GACCTGGATCCCGAGGACCCTGAGGTGCAGATCTCCT | |
| | | GGTTCGTGGACGGCAAGCAGATGCAGACCGCTAAAA | |
| | | CACAGCCTCGCGAGGAGCAGTTTAATGGTACCTACAG | |
| | | AGTGGTCTCTGTGCTGCCTATCGGCCACCAGGACTGG | |
| | | CTGAAAGGAAAACAGTTCACCTGCAAGGTGAACAAC | |
| | | AAGGCCCTGCCTTCTCCCATCGAGCGGACCATCTCCA | |
| | | AGGCTAGAGGCCAGGCCCATCAGCCTTCTGTGTACGT | |
| | | GCTGCCCCCATCCCGGGAAGAGCTGTCCAAGAACAC | |
| | | CGTGTCCCTCACCTGTCTGATCAAGGACTTCTTCCCC | |
| | | CCCGACATCGACGTCGAATGGCAGTCTAATGGCCAGC | |
| | | AGGAACCCGAGTCCAAGTACCGGACCACCCCTCCTC | |
| | | AGCTGGACGAGGATGGCTCCTACTTCCTGTACTCCAA | |
| | | GCTGTCTGTGGACAAGTCCAGGTGGCAGCGGGGCGA | |
| | | TACATTCATCTGCGCCGTGATGCACGAGGCCCTGCAC | |
| | | AACCACTACACTCAAGAGTCCCTGTCTCACTCCCCTG | |
| | | GATGA | |
| | VL | GCCGACATCGTGATGACCCAGACCCCTGCCTCTGTGG | |
| | | AAGCCGCCGTGGGCGGCATCGTGATCATCAAGTGCC | |
| | | AGGCCTCCGAGTCCATCGGCAACACCCTGGCTTGGTA | |
| | | TCAGCAGAAGCCCGGTCAGCCTCCAAAGCTGCTGAT | |
| | | CTACGGCGCTAGCACCCTGGCCTCTGGCGTGTCCAGC | |
| | | AGATTCAAGGGCTCTGGCTCCGGCACCGAGGTGACA | SEQ ID |
| | | CTGACCATCTCCGACCTGGAATGTGCCGATGCTGCTA | NO: |
| | | CCTACTACTGCCAGTCCTACTGGTCCTCTACCACCAC | 174 |
| | | ATTTCGGGACACCTTCGGCGGAGGAACAGAGGTCGT | |
| | | GGTGAAACGGAACGACGCCCAGCCTGCCGTGTACCT | |
| | | GTTCCAGCCCTCTCCAGATCAGCTGCACACAGGATCT | |
| | | GCTTCTGTGGTGTGCCTGCTGAACTCCTTCTACCCTA | |
| | | AAGACATCAACGTGAAGTGGAAGGTGGACGGCGTGA | |
| | | TCCAGGATACCGGCATCCAAGAGTCCGTGACCGAAC | |
| | | AGGACAAGGACTCCACCTACTCTCTGTCCAGCACAC | |
| | | TGACCATGTCCAGCACCGAGTATCTCAGCCATGAACT | |
| | | GTACTCCTGCGAGATCACCCACAAGTCCCTGCCTTCT | |
| | | ACCCTGATCAAGTCTTTTCAGAGATCCGAGTGTTGA | |
| Caninized | VH | GAGGTGCAGCTGGTGGAGAGCGGCGGCGACCTGGTG | SEQ ID |
| 38E2-H3L3 | | AAGCCCGGCGGCAGCCTGCGGCTGAGCTGCGTGGCC | NO: |
| antibody | | AGCGCCTTCAGCCTGAGCGGCTACTACATGGGCTGGG | 187 |
| | | TGCGGCAGGCCCCCGGCAAGGGCCTGGAGTGGATCA | |
| | | GCATCATCGGCGGCAGCAGCAACACCTTCTACGCCA | |
| | | GCTGGGCCAAGGGCCGGTTCACCATCAGCAAGGACA | |
| | | ACAGCAAGAACACCCTGTACCTGCAGATGAACAGCC | |
| | | TGCGGGCCGAGGACACCGCCGTGTACTTCTGCGCCC | |
| | | GGCACGGCGACACCGACTACATCAGCGGCACCTACT | |
| | | TCGACCTGTGGGGCCAGGGCACCCTGGTGACCGTGA | |
| | | GCAGCGCCAGCACCACCGCCCCCAGCGTGTTCCCCC | |
| | | TGGCCCCCAGCTGCGGCAGCACCAGCGGCAGCACCG | |
| | | TGGCCCTGGCCTGCCTGGTGAGCGGCTACTTCCCCGA | |
| | | GCCCGTGACCGTGAGCTGGAACAGCGGCAGCCTGAC | |
| | | CAGCGGCGTGCACACCTTCCCCAGCGTGCTGCAGAG | |
| | | CAGCGGCCTGTACAGCCTGAGCAGCATGGTGACCGT | |
| | | GCCCAGCAGCCGGTGGCCCAGCGAGACCTTCACCTG | |
| | | CAACGTGGCCCACCCCGCCAGCAAGACCAAGGTGGA | |
| | | CAAGCCCGTGCCCAAGCGGGAGAACGGCCGGGTGCC | |
| | | CCGGCCCCCCGACTGCCCCAAGTGCCCCGCCCCCGA | |
| | | GATGCTGGGCGGCCCCAGCGTGTTCATCTTCCCCCCC | |
| | | AAGCCCAAGGACACCCTGCTGATCGCCCGGACCCCC | |
| | | GAGGTGACCTGCGTGGTGGTGGACCTGGACCCCGAG | |
| | | GACCCCGAGGTGCAGATCAGCTGGTTCGTGGACGGC | |
| | | AAGCAGATGCAGACCGCCAAGACCCAGCCCCGGGAG | |
| | | GAGCAGTTCAACGGCACCTACCGGGTGGTGAGCGTG | |
| | | CTGCCCATCGGCCACCAGGACTGGCTGAAGGGCAAG | |
| | | CAGTTCACCTGCAAGGTGAACAACAAGGCCCTGCCC | |
| | | AGCCCCATCGAGCGGACCATCAGCAAGGCCCGGGGC | |
| | | CAGGCCCACCAGCCCAGCGTGTACGTGCTGCCCCCC | |
| | | AGCCGGGAGGAGCTGAGCAAGAACACCGTGAGCCT | |
| | | GACCTGCCTGATCAAGGACTTCTTCCCCCCCGACATC | |
| | | GACGTGGAGTGGCAGAGCAACGGCCAGCAGGAGCC | |
| | | CGAGAGCAAGTACCGGACCACCCCCCCCCAGCTGGA | |
| | | CGAGGACGGCAGCTACTTCCTGTACAGCAAGCTGAG | |
| | | CGTGGACAAGAGCCGGTGGCAGCGGGGCGACACCTT | |
| | | CATCTGCGCCGTGATGCACGAGGCCCTGCACAACCA | |
| | | CTACACCCAGGAGAGCCTGAGCCACAGCCCCGGC | |
| | VL | GAGATCGTGATGACCCAGAGCCCCGCCAGCCTGAGC | SEQ ID |
| | | CTGAGCCAGGGCGAGAAGGTGACCATCACCTGCCAG | NO: |
| | | GCCAGCCAGAGCATCAGCATCTACCTGGCCTGGTACC | 193 |
| | | AGCAGAAGCCCGGCCAGCCCCCCAAGCTGCTGATCT | |
| | | ACCGGGCCAGCACCCTGGCCAGCGGCGTGCCCAGCC | |
| | | GGTTCAGCGGCAGCGGCAGCGGCACCGACTTCAGCT | |
| | | TCACCATCACCAGCCTGCAGCCCGAGGACGTGGCCA | |
| | | CCTACTACTGCCAGCAGGGCTACGAGGACAGCAACG | |
| | | TGGAGAGCCTGTTCGGCCAGGGCACCAAGGTGGAGA | |
| | | TCAAGCGGAACGACGCCCAGCCCGCCGTGTACCTGT | |
| | | TCCAGCCCAGCCCCGACCAGCTGCACACCGGCAGCG | |
| | | CCAGCGTGGTGTGCCTGCTGAACAGCTTCTACCCCAA | |
| | | GGACATCAACGTGAAGTGGAAGGTGGACGGCGTGAT | |
| | | CCAGGACACCGGCATCCAGGAGAGCGTGACCGAGCA | |
| | | GGACAAGGACAGCACCTACAGCCTGAGCAGCACCCT | |
| | | GACCATGAGCAGCACCGAGTACCTGAGCCACGAGCT | |
| | | GTACAGCTGCGAGATCACCCACAAGAGCCTGCCCAG | |
| | | CACCCTGATCAAGAGCTTCCAGCGGAGCGAGTGC | |
| Caninized | VH | GAGGTGCAGCTGGTGGAGAGCGGCGGCGACCTGGTG | SEQ ID |
| 38E2-H3L2 | | AAGCCCGGCGGCAGCCTGCGGCTGAGCTGCGTGGCC | NO: |
| antibody | | AGCGCCTTCAGCCTGAGCGGCTACTACATGGGCTGGG | 188 |
| | | TGCGGCAGGCCCCCGGCAAGGGCCTGGAGTGGATCA | |
| | | GCATCATCGGCGGCAGCAGCAACACCTTCTACGCCA | |
| | | GCTGGGCCAAGGGCCGGTTCACCATCAGCAAGGACA | |
| | | ACAGCAAGAACACCCTGTACCTGCAGATGAACAGCC | |
| | | TGCGGGCCGAGGACACCGCCGTGTACTTCTGCGCCC | |
| | | GGCACGGCGACACCGACTACATCAGCGGCACCTACT | |
| | | TCGACCTGTGGGGCCAGGGCACCCTGGTGACCGTGA | |
| | | GCAGCGCCAGCACCACCGCCCCCAGCGTGTTCCCCC | |
| | | TGGCCCCCAGCTGCGGCAGCACCAGCGGCAGCACCG | |
| | | TGGCCCTGGCCTGCCTGGTGAGCGGCTACTTCCCCGA | |
| | | GCCCGTGACCGTGAGCTGGAACAGCGGCAGCCTGAC | |
| | | CAGCGGCGTGCACACCTTCCCCAGCGTGCTGCAGAG | |
| | | CAGCGGCCTGTACAGCCTGAGCAGCATGGTGACCGT | |
| | | GCCCAGCAGCCGGTGGCCCAGCGAGACCTTCACCTG | |
| | | CAACGTGGCCCACCCCGCCAGCAAGACCAAGGTGGA | |
| | | CAAGCCCGTGCCCAAGCGGGAGAACGGCCGGGTGCC | |
| | | CCGGCCCCCCGACTGCCCCAAGTGCCCCGCCCCCGA | |
| | | GATGCTGGGCGGCCCCAGCGTGTTCATCTTCCCCCCC | |
| | | AAGCCCAAGGACACCCTGCTGATCGCCCGGACCCCC | |
| | | GAGGTGACCTGCGTGGTGGTGGACCTGGACCCCGAG | |
| | | GACCCCGAGGTGCAGATCAGCTGGTTCGTGGACGGC | |
| | | AAGCAGATGCAGACCGCCAAGACCCAGCCCCGGGAG | |
| | | GAGCAGTTCAACGGCACCTACCGGGTGGTGAGCGTG | |
| | | CTGCCCATCGGCCACCAGGACTGGCTGAAGGGCAAG | |
| | | CAGTTCACCTGCAAGGTGAACAACAAGGCCCTGCCC | |
| | | AGCCCCATCGAGCGGACCATCAGCAAGGCCCGGGGC | |
| | | CAGGCCCACCAGCCCAGCGTGTACGTGCTGCCCCCC | |
| | | AGCCGGGAGGAGCTGAGCAAGAACACCGTGAGCCT | |
| | | GACCTGCCTGATCAAGGACTTCTTCCCCCCCGACATC | |
| | | GACGTGGAGTGGCAGAGCAACGGCCAGCAGGAGCC | |
| | | CGAGAGCAAGTACCGGACCACCCCCCCCCAGCTGGA | |
| | | CGAGGACGGCAGCTACTTCCTGTACAGCAAGCTGAG | |
| | | CGTGGACAAGAGCCGGTGGCAGCGGGGCGACACCTT | |
| | | CATCTGCGCCGTGATGCACGAGGCCCTGCACAACCA | |
| | | CTACACCCAGGAGAGCCTGAGCCACAGCCCCGGC | |
| | VL | GAGATCGTGATGACCCAGAGCCCCGCCAGCCTGAGC | SEQ ID |
| | | CTGAGCCAGGGCGAGAAGGTGACCATCACCTGCCAG | NO: |
| | | GCCAGCCAGAGCATCAGCATCTACCTGGCCTGGTACC | 194 |
| | | AGCAGAAGCCCGGCCAGCCCCCCAAGCTGCTGATCT | |
| | | ACCGGGCCAGCACCCTGGCCAGCGGCGTGCCCAGCC | |
| | | GGTTCAGCGGCAGCGGCAGCGGCACCGACTTCAGCT | |
| | | TCACCATCAGCAGCCTGCAGCCCGAGGACGTGGCCA | |
| | | CCTACTACTGCCAGCAGGGCTACGAGGACAGCAACG | |
| | | TGGAGAGCCTGTTCGGCCAGGGCACCAAGGTGGAGA | |
| | | TCAAGCGGAACGACGCCCAGCCCGCCGTGTACCTGT | |
| | | TCCAGCCCAGCCCCGACCAGCTGCACACCGGCAGCG | |
| | | CCAGCGTGGTGTGCCTGCTGAACAGCTTCTACCCCAA | |
| | | GGACATCAACGTGAAGTGGAAGGTGGACGGCGTGAT | |
| | | CCAGGACACCGGCATCCAGGAGAGCGTGACCGAGCA | |
| | | GGACAAGGACAGCACCTACAGCCTGAGCAGCACCCT | |
| | | GACCATGAGCAGCACCGAGTACCTGAGCCACGAGCT | |
| | | GTACAGCTGCGAGATCACCCACAAGAGCCTGCCCAG | |
| | | CACCCTGATCAAGAGCTTCCAGCGGAGCGAGTGC | |
| Caninized | VH | ATGGAGACCGGCCTGCGGTGGCTGCTGCTGGTGGCC | SEQ ID |
| 47C1-1-H2L2 | | GTGCTGAAGGGCGTGCAGTGCGAGGTGCAGCTGGTG | NO: |
| antibody | | GAGAGCGGCGGCGACCTGGTGAAGCCCGGCGGCAG | 189 |
| | | CCTGCGGCTGAGCTGCGTGGCCAGCGGCTTCAGCCT | |
| | | GAGCACCTACGCCATGAACTGGGTGCGGCAGGCCCC | |
| | | CGGCAAGGGCCTGGAGTGGATCAGCATCATCTACAG | |
| | | CAACGGCGACGGCTACTACGCCAGCTGGGCCAAGGG | |
| | | CCGGTTCACCATCAGCCGGGACAACGCCAAGAACAC | |
| | | CCTGTACCTGCAGATGAACAGCCTGCGGGCCGAGGA | |
| | | CACCGCCGTGTACTACTGCGCCCGGGGCATCTACAGC | |
| | | CAGACCACCGGCTACTTCAACCTGTGGGGCCAGGGC | |
| | | ACCCTGGTGACCGTGAGCAGCGCCAGCACCACCGCC | |
| | | CCCAGCGTGTTCCCCCTGGCCCCCAGCTGCGGCAGC | |
| | | ACCAGCGGCAGCACCGTGGCCCTGGCCTGCCTGGTG | |
| | | AGCGGCTACTTCCCCGAGCCCGTGACCGTGAGCTGG | |
| | | AACAGCGGCAGCCTGACCAGCGGCGTGCACACCTTC | |
| | | CCCAGCGTGCTGCAGAGCAGCGGCCTGTACAGCCTG | |
| | | AGCAGCATGGTGACCGTGCCCAGCAGCCGGTGGCCC | |
| | | AGCGAGACCTTCACCTGCAACGTGGCCCACCCCGCC | |
| | | AGCAAGACCAAGGTGGACAAGCCCGTGCCCAAGCG | |
| | | GGAGAACGGCCGGGTGCCCCGGCCCCCCGACTGCCC | |
| | | CAAGTGCCCCGCCCCCGAGATGCTGGGCGGCCCCAG | |
| | | CGTGTTCATCTTCCCCCCCAAGCCCAAGGACACCCTG | |
| | | CTGATCGCCCGGACCCCCGAGGTGACCTGCGTGGTG | |
| | | GTGGACCTGGACCCCGAGGACCCCGAGGTGCAGATC | |
| | | AGCTGGTTCGTGGACGGCAAGCAGATGCAGACCGCC | |
| | | AAGACCCAGCCCCGGGAGGAGCAGTTCAACGGCACC | |
| | | TACCGGGTGGTGAGCGTGCTGCCCATCGGCCACCAG | |
| | | GACTGGCTGAAGGGCAAGCAGTTCACCTGCAAGGTG | |
| | | AACAACAAGGCCCTGCCCAGCCCCATCGAGCGGACC | |
| | | ATCAGCAAGGCCCGGGGCCAGGCCCACCAGCCCAGC | |
| | | GTGTACGTGCTGCCCCCCAGCCGGGAGGAGCTGAGC | |
| | | AAGAACACCGTGAGCCTGACCTGCCTGATCAAGGAC | |
| | | TTCTTCCCCCCCGACATCGACGTGGAGTGGCAGAGC | |
| | | AACGGCCAGCAGGAGCCCGAGAGCAAGTACCGGAC | |
| | | CACCCCCCCCCAGCTGGACGAGGACGGCAGCTACTT | |
| | | CCTGTACAGCAAGCTGAGCGTGGACAAGAGCCGGTG | |
| | | GCAGCGGGGCGACACCTTCATCTGCGCCGTGATGCA | |
| | | CGAGGCCCTGCACAACCACTACACCCAGGAGAGCCT | |
| | | GAGCCACAGCCCCGGC | |
| | VL | ATGGACACCCGGGCCCCCACCCAGCTGCTGGGCCTG | SEQ ID |
| | | CTGCTGCTGTGGCTGCCCGGCGCCCGGTGCGAGATC | NO: |
| | | GTGATGACCCAGACCCCCGCCAGCCTGAGCCTGAGC | 195 |
| | | CAGGAGGGCAAGGTGACCATCACCTGCCAGGCCAGC | |
| | | GAGAGCATCGGCAACACCCTGGCCTGGTACCAGCAG | |
| | | AAGCCCGGCCAGCCCCCCAAGCTGCTGATCTACGGC | |
| | | GCCAGCACCCTGGCCAGCGGCGTGCCCAGCCGGTTC | |
| | | AGCGGCAGCGGCAGCGGCACCGACTTCACCTTCACC | |
| | | ATCAACAGCCTGCAGCCCGAGGACGTGGCCACCTAC | |
| | | TACTGCCAGAGCTACTGGAGCAGCACCACCACCTTCC | |
| | | GGGACACCTTCGGCCAGGGCACCAAGGTGGAGATCA | |
| | | AGCGGAACGACGCCCAGCCCGCCGTGTACCTGTTCC | |
| | | AGCCCAGCCCCGACCAGCTGCACACCGGCAGCGCCA | |
| | | GCGTGGTGTGCCTGCTGAACAGCTTCTACCCCAAGG | |
| | | ACATCAACGTGAAGTGGAAGGTGGACGGCGTGATCC | |
| | | AGGACACCGGCATCCAGGAGAGCGTGACCGAGCAG | |
| | | GACAAGGACAGCACCTACAGCCTGAGCAGCACCCTG | |
| | | ACCATGAGCAGCACCGAGTACCTGAGCCACGAGCTG | |
| | | TACAGCTGCGAGATCACCCACAAGAGCCTGCCCAGC | |
| | | ACCCTGATCAAGAGCTTCCAGCGGAGCGAGTGC | |
| Caninized | VH | ATGGAGACCGGCCTGCGGTGGCTGCTGCTGGTGGCC | SEQ ID |
| 47C1-1-H3L3 | | GTGCTGAAGGGCGTGCAGTGCGAGGTGCAGCTGGTG | NO: |
| antibody | | GAGAGCGGCGGCGACCTGGTGAAGCCCGGCGGCAG | 190 |
| | | CCTGCGGCTGAGCTGCGTGGCCAGCGGCTTCAGCCT | |
| | | GAGCACCTACGCCATGAACTGGGTGCGGCAGGCCCC | |
| | | CGGCAAGGGCCTGGAGTGGATCAGCATCATCTACAG | |
| | | CAACGGCGACGGCTACTACGCCAGCTGGGCCAAGGG | |
| | | CCGGTTCACCATCAGCCGGGACAACAGCAAGAACAC | |
| | | CCTGTACCTGCAGATGAACAGCCTGCGGGCCGAGGA | |
| | | CACCGCCGTGTACTACTGCGCCCGGGGCATCTACAGC | |
| | | CAGACCACCGGCTACTTCAACCTGTGGGGCCAGGGC | |
| | | ACCCTGGTGACCGTGAGCAGCGCCAGCACCACCGCC | |
| | | CCCAGCGTGTTCCCCCTGGCCCCCAGCTGCGGCAGC | |
| | | ACCAGCGGCAGCACCGTGGCCCTGGCCTGCCTGGTG | |
| | | AGCGGCTACTTCCCCGAGCCCGTGACCGTGAGCTGG | |
| | | AACAGCGGCAGCCTGACCAGCGGCGTGCACACCTTC | |
| | | CCCAGCGTGCTGCAGAGCAGCGGCCTGTACAGCCTG | |
| | | AGCAGCATGGTGACCGTGCCCAGCAGCCGGTGGCCC | |
| | | AGCGAGACCTTCACCTGCAACGTGGCCCACCCCGCC | |
| | | AGCAAGACCAAGGTGGACAAGCCCGTGCCCAAGCG | |
| | | GGAGAACGGCCGGGTGCCCCGGCCCCCCGACTGCCC | |
| | | CAAGTGCCCCGCCCCCGAGATGCTGGGCGGCCCCAG | |
| | | CGTGTTCATCTTCCCCCCCAAGCCCAAGGACACCCTG | |
| | | CTGATCGCCCGGACCCCCGAGGTGACCTGCGTGGTG | |
| | | GTGGACCTGGACCCCGAGGACCCCGAGGTGCAGATC | |
| | | AGCTGGTTCGTGGACGGCAAGCAGATGCAGACCGCC | |
| | | AAGACCCAGCCCCGGGAGGAGCAGTTCAACGGCACC | |
| | | TACCGGGTGGTGAGCGTGCTGCCCATCGGCCACCAG | |
| | | GACTGGCTGAAGGGCAAGCAGTTCACCTGCAAGGTG | |
| | | AACAACAAGGCCCTGCCCAGCCCCATCGAGCGGACC | |
| | | ATCAGCAAGGCCCGGGGCCAGGCCCACCAGCCCAGC | |
| | | GTGTACGTGCTGCCCCCCAGCCGGGAGGAGCTGAGC | |
| | | AAGAACACCGTGAGCCTGACCTGCCTGATCAAGGAC | |
| | | TTCTTCCCCCCCGACATCGACGTGGAGTGGCAGAGC | |
| | | AACGGCCAGCAGGAGCCCGAGAGCAAGTACCGGAC | |
| | | CACCCCCCCCCAGCTGGACGAGGACGGCAGCTACTT | |
| | | CCTGTACAGCAAGCTGAGCGTGGACAAGAGCCGGTG | |
| | | GCAGCGGGGCGACACCTTCATCTGCGCCGTGATGCA | |
| | | CGAGGCCCTGCACAACCACTACACCCAGGAGAGCCT | |
| | | GAGCCACAGCCCCGGC | |
| | VL | ATGGACACCCGGGCCCCCACCCAGCTGCTGGGCCTG | SEQ ID |
| | | CTGCTGCTGTGGCTGCCCGGCGCCCGGTGCGAGATC | NO: |
| | | GTGATGACCCAGACCCCCGCCAGCCTGAGCCTGAGC | 196 |
| | | CAGGAGGGCAAGGTGACCATCACCTGCCAGGCCAGC | |
| | | GAGAGCATCGGCAACACCCTGGCCTGGTACCAGCAG | |
| | | AAGCCCGGCCAGCCCCCCAAGCTGCTGATCTACGGC | |
| | | GCCAGCACCCTGGCCAGCGGCGTGCCCAGCCGGTTC | |
| | | AGCGGCAGCGGCAGCGGCACCGACTTCACCTTCACC | |
| | | ATCAACAGCCTGCAGCCCGAGGACGTGGCCACCTAC | |
| | | TACTGCCAGAGCTACTGGAGCAGCACCACCACCTTCC | |
| | | GGGACACCTTCGGCCAGGGCACCAAGGTGGAGATCA | |
| | | AGCGGAACGACGCCCAGCCCGCCGTGTACCTGTTCC | |
| | | AGCCCAGCCCCGACCAGCTGCACACCGGCAGCGCCA | |
| | | GCGTGGTGTGCCTGCTGAACAGCTTCTACCCCAAGG | |
| | | ACATCAACGTGAAGTGGAAGGTGGACGGCGTGATCC | |
| | | AGGACACCGGCATCCAGGAGAGCGTGACCGAGCAG | |
| | | GACAAGGACAGCACCTACAGCCTGAGCAGCACCCTG | |
| | | ACCATGAGCAGCACCGAGTACCTGAGCCACGAGCTG | |
| | | TACAGCTGCGAGATCACCCACAAGAGCCTGCCCAGC | |
| | | ACCCTGATCAAGAGCTTCCAGCGGAGCGAGTGC | |
| Caninized | VH | GAGGTGCAGCTGGTGGAGAGCGGCGGCGACCTGGTG | SEQ ID |
| 35C9-1-H1L1 | | AAGCCCGGCGGCAGCCTGCGGCTGAGCTGCGTGGCC | NO: |
| antibody | | AGCGGCTTCACCTTCAGCAGCTACTGGATGAGCTGG | 191 |
| | | GTGCGGCAGGCCCCCGGCAAGGGCCTGCAGTGGCTG | |
| | | AGCGACATCTACGCCACCGGCAGCACCTACTACGCCA | |
| | | ACTGGGCCACCGGCCGGTTCACCGGCAGCCGGGACA | |
| | | ACGCCAAGAACACCCTGTACCTGCAGATGAACAGCC | |
| | | TGACCATCGAGGACACCGCCACCTACTTCTGCGCCCG | |
| | | GCACAGCAGCCGGAGCGGCATCGACTTCGACCTGTG | |
| | | GGGCCAGGGCACCCTGGTGACCGTGAGCAGCGCCAG | |
| | | CACCACCGCCCCCAGCGTGTTCCCCCTGGCCCCCAGC | |
| | | TGCGGCAGCACCAGCGGCAGCACCGTGGCCCTGGCC | |
| | | TGCCTGGTGAGCGGCTACTTCCCCGAGCCCGTGACC | |
| | | GTGAGCTGGAACAGCGGCAGCCTGACCAGCGGCGTG | |
| | | CACACCTTCCCCAGCGTGCTGCAGAGCAGCGGCCTG | |
| | | TACAGCCTGAGCAGCATGGTGACCGTGCCCAGCAGC | |
| | | CGGTGGCCCAGCGAGACCTTCACCTGCAACGTGGCC | |
| | | CACCCCGCCAGCAAGACCAAGGTGGACAAGCCCGTG | |
| | | CCCAAGCGGGAGAACGGCCGGGTGCCCCGGCCCCCC | |
| | | GACTGCCCCAAGTGCCCCGCCCCCGAGATGCTGGGC | |
| | | GGCCCCAGCGTGTTCATCTTCCCCCCCAAGCCCAAGG | |
| | | ACACCCTGCTGATCGCCCGGACCCCCGAGGTGACCT | |
| | | GCGTGGTGGTGGACCTGGACCCCGAGGACCCCGAGG | |
| | | TGCAGATCAGCTGGTTCGTGGACGGCAAGCAGATGC | |
| | | AGACCGCCAAGACCCAGCCCCGGGAGGAGCAGTTCA | |
| | | ACGGCACCTACCGGGTGGTGAGCGTGCTGCCCATCG | |
| | | GCCACCAGGACTGGCTGAAGGGCAAGCAGTTCACCT | |
| | | GCAAGGTGAACAACAAGGCCCTGCCCAGCCCCATCG | |
| | | AGCGGACCATCAGCAAGGCCCGGGGCCAGGCCCACC | |
| | | AGCCCAGCGTGTACGTGCTGCCCCCCAGCCGGGAGG | |
| | | AGCTGAGCAAGAACACCGTGAGCCTGACCTGCCTGA | |
| | | TCAAGGACTTCTTCCCCCCCGACATCGACGTGGAGTG | |
| | | GCAGAGCAACGGCCAGCAGGAGCCCGAGAGCAAGT | |
| | | ACCGGACCACCCCCCCCCAGCTGGACGAGGACGGCA | |
| | | GCTACTTCCTGTACAGCAAGCTGAGCGTGGACAAGA | |
| | | GCCGGTGGCAGCGGGGCGACACCTTCATCTGCGCCG | |
| | | TGATGCACGAGGCCCTGCACAACCACTACACCCAGG | |
| | | AGAGCCTGAGCCACAGCCCCGGC | |
| | VL | GAGATCGTGATGACCCAGAGCCCCGCCAGCCTGAGC | SEQ ID |
| | | CTGAGCCAGGAGGAGAAGGTGACCATCACCTGCCAG | NO: |
| | | GCCAGCCAGAGCATCGGCAACTACCTGGCCTGGTAC | 197 |
| | | CAGCAGAAGCCCGGCCAGGCCCCCAAGCAGCTGATC | |
| | | TACCGGGCCAGCACCCTGGCCAGCGGCGTGCCCAGC | |
| | | CGGTTCAAGGGCAGCGGCAGCGGCACCGAGTTCACC | |
| | | CTGACCATCAGCGACCTGGAGTGCGCCGACGTGGCC | |
| | | GTGTACTACTGCCAGCAGGGCTACGACAGCAACAAC | |
| | | GTGGACAACTTCTTCGGCCAGGGCACCAAGGTGGAG | |
| | | ATCAAGCGGAACGACGCCCAGCCCGCCGTGTACCTG | |
| | | TTCCAGCCCAGCCCCGACCAGCTGCACACCGGCAGC | |
| | | GCCAGCGTGGTGTGCCTGCTGAACAGCTTCTACCCCA | |
| | | AGGACATCAACGTGAAGTGGAAGGTGGACGGCGTGA | |
| | | TCCAGGACACCGGCATCCAGGAGAGCGTGACCGAGC | |
| | | AGGACAAGGACAGCACCTACAGCCTGAGCAGCACCC | |
| | | TGACCATGAGCAGCACCGAGTACCTGAGCCACGAGC | |
| | | TGTACAGCTGCGAGATCACCCACAAGAGCCTGCCCA | |
| | | GCACCCTGATCAAGAGCTTCCAGCGGAGCGAGTGC | |
| Caninized | VH | GAGGTGCAGCTGGTGGAGAGCGGCGGCGACCTGGTG | SEQ ID |
| 35C9-1-H1L2 | | AAGCCCGGCGGCAGCCTGCGGCTGAGCTGCGTGGCC | NO: |
| antibody | | AGCGGCTTCACCTTCAGCAGCTACTGGATGAGCTGG | 192 |
| | | GTGCGGCAGGCCCCCGGCAAGGGCCTGCAGTGGCTG | |
| | | AGCGACATCTACGCCACCGGCAGCACCTACTACGCCA | |
| | | ACTGGGCCACCGGCCGGTTCACCGGCAGCCGGGACA | |
| | | ACGCCAAGAACACCCTGTACCTGCAGATGAACAGCC | |
| | | TGACCATCGAGGACACCGCCACCTACTTCTGCGCCCG | |
| | | GCACAGCAGCCGGAGCGGCATCGACTTCGACCTGTG | |
| | | GGGCCAGGGCACCCTGGTGACCGTGAGCAGCGCCAG | |
| | | CACCACCGCCCCCAGCGTGTTCCCCCTGGCCCCCAGC | |
| | | TGCGGCAGCACCAGCGGCAGCACCGTGGCCCTGGCC | |
| | | TGCCTGGTGAGCGGCTACTTCCCCGAGCCCGTGACC | |
| | | GTGAGCTGGAACAGCGGCAGCCTGACCAGCGGCGTG | |
| | | CACACCTTCCCCAGCGTGCTGCAGAGCAGCGGCCTG | |
| | | TACAGCCTGAGCAGCATGGTGACCGTGCCCAGCAGC | |
| | | CGGTGGCCCAGCGAGACCTTCACCTGCAACGTGGCC | |
| | | CACCCCGCCAGCAAGACCAAGGTGGACAAGCCCGTG | |
| | | CCCAAGCGGGAGAACGGCCGGGTGCCCCGGCCCCCC | |
| | | GACTGCCCCAAGTGCCCCGCCCCCGAGATGCTGGGC | |
| | | GGCCCCAGCGTGTTCATCTTCCCCCCCAAGCCCAAGG | |
| | | ACACCCTGCTGATCGCCCGGACCCCCGAGGTGACCT | |
| | | GCGTGGTGGTGGACCTGGACCCCGAGGACCCCGAGG | |
| | | TGCAGATCAGCTGGTTCGTGGACGGCAAGCAGATGC | |
| | | AGACCGCCAAGACCCAGCCCCGGGAGGAGCAGTTCA | |
| | | ACGGCACCTACCGGGTGGTGAGCGTGCTGCCCATCG | |
| | | GCCACCAGGACTGGCTGAAGGGCAAGCAGTTCACCT | |
| | | GCAAGGTGAACAACAAGGCCCTGCCCAGCCCCATCG | |
| | | AGCGGACCATCAGCAAGGCCCGGGGCCAGGCCCACC | |
| | | AGCCCAGCGTGTACGTGCTGCCCCCCAGCCGGGAGG | |
| | | AGCTGAGCAAGAACACCGTGAGCCTGACCTGCCTGA | |
| | | TCAAGGACTTCTTCCCCCCCGACATCGACGTGGAGTG | |
| | | GCAGAGCAACGGCCAGCAGGAGCCCGAGAGCAAGT | |
| | | ACCGGACCACCCCCCCCCAGCTGGACGAGGACGGCA | |
| | | GCTACTTCCTGTACAGCAAGCTGAGCGTGGACAAGA | |
| | | GCCGGTGGCAGCGGGGCGACACCTTCATCTGCGCCG | |
| | | TGATGCACGAGGCCCTGCACAACCACTACACCCAGG | |
| | | AGAGCCTGAGCCACAGCCCCGGC | |
| | VL | GAGATCGTGATGACCCAGAGCCCCGCCAGCCTGAGC | SEQ ID |
| | | CTGAGCCAGGAGGAGAAGGTGACCATCACCTGCCAG | NO: |
| | | GCCAGCCAGAGCATCGGCAACTACCTGGCCTGGTAC | 198 |
| | | CAGCAGAAGCCCGGCCAGGCCCCCAAGCAGCTGATC | |
| | | TACCGGGCCAGCACCCTGGCCAGCGGCGTGCCCAGC | |
| | | CGGTTCAGCGGCAGCGGCAGCGGCACCGACTTCACC | |
| | | CTGACCATCAGCGACCTGGAGTGCGCCGACGTGGCC | |
| | | GTGTACTACTGCCAGCAGGGCTACGACAGCAACAAC | |
| | | GTGGACAACTTCTTCGGCCAGGGCACCAAGGTGGAG | |
| | | ATCAAGCGGAACGACGCCCAGCCCGCCGTGTACCTG | |
| | | TTCCAGCCCAGCCCCGACCAGCTGCACACCGGCAGC | |
| | | GCCAGCGTGGTGTGCCTGCTGAACAGCTTCTACCCCA | |
| | | AGGACATCAACGTGAAGTGGAAGGTGGACGGCGTGA | |
| | | TCCAGGACACCGGCATCCAGGAGAGCGTGACCGAGC | |
| | | AGGACAAGGACAGCACCTACAGCCTGAGCAGCACCC | |
| | | TGACCATGAGCAGCACCGAGTACCTGAGCCACGAGC | |
| | | TGTACAGCTGCGAGATCACCCACAAGAGCCTGCCCA | |
| | | GCACCCTGATCAAGAGCTTCCAGCGGAGCGAGTGC | |
| Canine IL-31 | | GCCCCCACCCACCAGCTGCCCCCCAGCGACGTGCGG | SEQ ID |
| | | AAGATCATCCTGGAGCTGCAGCCCCTGAGCCGGGGC | NO: |
| | | CTGCTGGAGGACTACCAGAAGAAGGAGACCGGCGTG | 200 |
| | | CCCGAGAGCAACCGGACCCTGCTGCTGTGCCTGACC | |
| | | AGCGACAGCCAGCCCCCCCGGCTGAACAGCAGCGCC | |
| | | ATCCTGCCCTACTTCCGGGCCATCCGGCCCCTGAGCG | |
| | | ACAAGAACATCATCGACAAGATCATCGAGCAGCTGG | |
| | | ACAAGCTGAAGTTCCAGCACGAGCCCGAGACCGAGA | |
| | | TCAGCGTGCCCGCCGACACCTTCGAGTGCAAGAGCT | |
| | | TCATCCTGACCATCCTGCAGCAGTTCAGCGCCTGCCT | |
| | | GGAGAGCGTGTTCAAGAGCCTGAACAGCGGCCCCCA | |
| | | GCACCACCACCACCACCACTGA | |

What is claimed is:

1. An antibody that specifically binds canine IL-31 or an antigen-binding fragment thereof, comprising a heavy chain variable region and a light chain variable region, wherein:
the heavy chain variable region comprises: HCDR1 having the amino acid sequence set forth in SEQ ID NO: 3; HCDR2 having the amino acid sequence set forth in SEQ ID NO: 13; and HCDR3 having the amino acid sequence set forth in SEQ ID NO: 23;
the light chain variable region comprises: LCDR1 having the amino acid sequence set forth in SEQ ID NO: 33; LCDR2 having the amino acid sequence set forth in SEQ ID NO: 43; and LCDR3 having the amino acid sequence set forth in SEQ ID NO: 53.

2. The antibody or antigen-binding fragment thereof according to claim 1, wherein the antibody or antigen-binding fragment thereof is selected from any one of a rabbit-derived antibody or antigen-binding fragment thereof, a chimeric antibody or antigen-binding fragment thereof, or a caninized antibody or antigen-binding fragment thereof.

3. The antibody or antigen-binding fragment thereof according to claim 1, wherein the antibody that specifically binds canine IL-31 or antigen-binding fragment thereof comprises a heavy chain selected from the sequences set forth as follows, or a heavy chain having at least 80% identity as compared to the following sequences: SEQ ID NOS: 63, 163, 175 or 176; and/or
comprises a light chain selected from the sequences set forth as follows, or a light chain having at least 80% identity as compared to the following sequences: SEQ ID NOS: 73, 166, 181 or 182.

4. The antibody or antigen-binding fragment thereof according to claim 1, wherein the antibody that specifically binds canine IL-31 or antigen-binding fragment thereof comprises a heavy chain selected from the sequences set forth as follows, or a heavy chain having at least 85% identity as compared to the following sequences: SEQ ID NOS: 63, 163, 175 or 176; and/or
comprises a light chain selected from the sequences set forth as follows, or a light chain having at least 85% identity as compared to the following sequences: SEQ ID NOS: 73, 166, 181 or 182.

5. The antibody or antigen-binding fragment thereof according to claim 1, wherein the antibody that specifically binds canine IL-31 or antigen-binding fragment thereof comprises a heavy chain selected from the sequences set forth as follows, or a heavy chain having at least 90% identity as compared to the following sequences: SEQ ID NOS: 63, 163, 175 or 176; and/or
comprises a light chain selected from the sequences set forth as follows, or a light chain having at least 90% identity as compared to the following sequences: SEQ ID NOS: 73, 166, 181 or 182.

6. The antibody or antigen-binding fragment thereof according to claim 1, wherein the antibody that specifically binds canine IL-31 or antigen-binding fragment thereof comprises a heavy chain selected from the sequences set forth as follows, or a heavy chain having at least 95% identity as compared to the following sequences: SEQ ID NOS: 63, 163, 175 or 176; and/or
comprises a light chain selected from the sequences set forth as follows, or a light chain having at least 95% identity as compared to the following sequences: SEQ ID NOS: 73, 166, 181 or 182.

7. The antibody or antigen-binding fragment thereof according to claim 1, wherein the antibody that specifically binds canine IL-31 or antigen-binding fragment thereof comprises a heavy chain selected from the sequences set forth as follows, or a heavy chain having at least 99% identity as compared to the following sequences: SEQ ID NOS: 63, 163, 175 or 176; and/or
comprises a light chain selected from the sequences set forth as follows, or a light chain having at least 99% identity as compared to the following sequences: SEQ ID NOS: 73, 166, 181 or 182.

8. The antibody or antigen-binding fragment thereof according to claim 3, wherein:
(1) the heavy chain comprises the amino acid sequence set forth in SEQ ID NO: 63 or any variant thereof; the light chain comprises the amino acid sequence set forth in SEQ ID NO: 73 or any variant thereof; or,
(2) the heavy chain comprises the amino acid sequence set forth in SEQ ID NO: 163 or any variant thereof; the light chain comprises the amino acid sequence set forth in SEQ ID NO: 166 or any variant thereof; or,
(3) the heavy chain comprises the amino acid sequence set forth in SEQ ID NO: 175 or any variant thereof; the light chain comprises the amino acid sequence set forth in SEQ ID NO: 181 or any variant thereof; or,
(4) the heavy chain comprises the amino acid sequence set forth in SEQ ID NO: 176 or any variant thereof; the light chain comprises the amino acid sequence set forth in SEQ ID NO: 182 or any variant thereof.

9. A biological material selected from any one of the following:
(1) a polynucleotide encoding the antibody or antigen-binding fragment thereof according to claim 1; or
(2) an expression vector comprising the polynucleotide of (1); or
(3) a host cell comprising the polynucleotide of (1) or the expression vector of (2).

10. A pharmaceutical composition comprising the antibody or antigen-binding fragment thereof according to claim 1, as well as a pharmaceutically acceptable excipient, diluent or carrier.

11. A kit for detecting or quantifying canine IL-31 protein in a clinical or biological sample, comprising the antibody or antigen-binding fragment thereof according to claim 1, and optionally, one or more reagents for detecting the binding of the antibody or antigen-binding fragment thereof to canine IL-31 or an epitope thereof.

12. A use of the antibody or antigen-binding fragment thereof according to claim 1, a biological material, a pharmaceutical composition, or a kit in the manufacture of a medicament for treating pruritus or allergy in a canine; wherein the biological material is selected from: a polynucleotide encoding the antibody or antigen-binding fragment thereof, an expression vector comprising the polynucleotide, a host cell comprising the polynucleotide, or a host cell comprising the expression vector; the pharmaceutical composition comprises the antibody or antigen-binding fragment thereof, as well as a pharmaceutically acceptable excipient, diluent or carrier; and the kit is used for detecting or quantifying canine IL-31 protein in a clinical or biological sample and comprises the antibody or antigen-binding fragment thereof, and optionally, one or more reagents for detecting the binding of the antibody or antigen-binding fragment thereof to canine IL-31 or an epitope thereof.

13. The use according to claim 12, wherein the pruritus is atopic dermatitis, eczema, psoriasis, or scleroderma.

14. The use according to claim 12, wherein the pruritus is atopic dermatitis.


SEQUENCE LISTING (200 sequences)

SEQ ID NO: 1 - protein, 5 aa, synthetic construct
SYWMS

SEQ ID NO: 2 - protein, 5 aa, synthetic construct
TYAMN

SEQ ID NO: 3 - protein, 5 aa, synthetic construct
GYYMG

SEQ ID NO: 4 - protein, 6 aa, synthetic construct
SDYYMC

SEQ ID NO: 5 - protein, 5 aa, synthetic construct
SYWMS

SEQ ID NO: 6 - protein, 5 aa, synthetic construct
GYYMS

SEQ ID NO: 7 - protein, 5 aa, synthetic construct
SYAMG

SEQ ID NO: 8 - protein, 5 aa, synthetic construct
NSYMG

SEQ ID NO: 9 - protein, 5 aa, synthetic construct
GYYMN

SEQ ID NO: 10 - protein, 5 aa, synthetic construct
SYAMI

SEQ ID NO: 11 - protein, 16 aa, synthetic construct
DIYATGSTYYANWATG

SEQ ID NO: 12 - protein, 16 aa, synthetic construct
IIYSNGDGYYASWAKG

SEQ ID NO: 13 - protein, 16 aa, synthetic construct
IIGGSSNTFYASWAKG

SEQ ID NO: 14 - protein, 17 aa, synthetic construct
CITTSTVGTWYASWAKG

SEQ ID NO: 15 - protein, 16 aa, synthetic construct
DIYASGSEYYASWVNG

SEQ ID NO: 16 - protein, 16 aa, synthetic construct
IIGGSGNTFYASWAKG

SEQ ID NO: 17 - protein, 16 aa, synthetic construct
IINSSGSTWYATWVKG

SEQ ID NO: 18 - protein, 16 aa, synthetic construct
FIDTGGTTYYASWAKG

SEQ ID NO: 19 - protein, 16 aa, synthetic construct
FIYSPSTTYYANWAKG

SEQ ID NO: 20 - protein, 16 aa, synthetic construct
TIDPSDNTDYASWAKG

SEQ ID NO: 21 - protein, 11 aa, synthetic construct
HSSRSGIDFDL

SEQ ID NO: 22 - protein, 12 aa, synthetic construct
GIYSQTTGYFNL

SEQ ID NO: 23 - protein, 14 aa, synthetic construct
HGDTDYISGTYFDL

SEQ ID NO: 24 - protein, 11 aa, synthetic construct
EDGTYADYFNL

SEQ ID NO: 25 - protein, 11 aa, synthetic construct
HSSRSGIDFDL

SEQ ID NO: 26 - protein, 14 aa, synthetic construct
HGDTDYISGTYFDL

SEQ ID NO: 27 - protein, 12 aa, synthetic construct
NRDPFYITDMDL

SEQ ID NO: 28 - protein, 9 aa, synthetic construct
GPDDYFFNL

SEQ ID NO: 29 - protein, 10 aa, synthetic construct
GHTTEIRLDL

SEQ ID NO: 30 - protein, 13 aa, synthetic construct
HSYDDYGRDVWNL

SEQ ID NO: 31 - protein, 11 aa, synthetic construct
QASQSIGNYLA

SEQ ID NO: 32 - protein, 11 aa, synthetic construct
QASESIGNTLA

SEQ ID NO: 33 - protein, 11 aa, synthetic construct
QASQSISIYLA

SEQ ID NO: 34 - protein, 11 aa, synthetic construct
QASQSISSWLA

SEQ ID NO: 35 - protein, 11 aa, synthetic construct
QASQSISNYLA

SEQ ID NO: 36 - protein, 11 aa, synthetic construct
QASQSISIYLA

SEQ ID NO: 37 - protein, 11 aa, synthetic construct
QASQNIYSYLS

SEQ ID NO: 38 - protein, 11 aa, synthetic construct
QASESISSWLA

SEQ ID NO: 39 - protein, 13 aa, synthetic construct
QSSESVYKNNRLS

SEQ ID NO: 40 - protein, 13 aa, synthetic construct
QASQSVDNNNDLS

SEQ ID NO: 41 - protein, 7 aa, synthetic construct
RASTLAS

SEQ ID NO: 42 - protein, 7 aa, synthetic construct
GASTLAS

SEQ ID NO: 43 - protein, 7 aa, synthetic construct
RASTLAS

SEQ ID NO: 44 - protein, 7 aa, synthetic construct
RASTLAS

SEQ ID NO: 45 - protein, 7 aa, synthetic construct
RASTLAS

SEQ ID NO: 46 - protein, 7 aa, synthetic construct
KASTLAS

SEQ ID NO: 47 - protein, 7 aa, synthetic construct
RASTLAS

SEQ ID NO: 48 - protein, 7 aa, synthetic construct
KASTLES

SEQ ID NO: 49 - protein, 7 aa, synthetic construct
DASTLAS

SEQ ID NO: 50 - protein, 7 aa, synthetic construct
YTSSLPS

SEQ ID NO: 51 - protein, 12 aa, synthetic construct
QQGYDSNNVDNF

SEQ ID NO: 52 - protein, 13 aa, synthetic construct
QSYWSSTTTFRDT

SEQ ID NO: 53 - protein, 12 aa, synthetic construct
QQGYEDSNVESL

SEQ ID NO: 54 - protein, 12 aa, synthetic construct
QQGYRSSNLDNA

SEQ ID NO: 55 - protein, 12 aa, synthetic construct
QQGYDSNNVDNL

SEQ ID NO: 56 - protein, 12 aa, synthetic construct
QQGYEDSNVESL

SEQ ID NO: 57 - protein, 12 aa, synthetic construct
QQGDSYSDVDNI

SEQ ID NO: 58 - protein, 12 aa, synthetic construct
QVYYFSYNSYNF

SEQ ID NO: 59 - protein, 11 aa, synthetic construct
LGEFRDSSDNS

SEQ ID NO: 60 - protein, 12 aa, synthetic construct
VGYKSSTTDATA

SEQ ID NO: 61 - protein, 116 aa, synthetic construct
QSLEESGGRLVTPGTPLTLTCTASGFSLSSYWMSWVRQAPGKGLEWIGDIYATGSTYYANWATGRFTGSKTSTTVDLTITSPTIEDTATYFCARHSSRSGIDFDLWGQGTLVTVSS

SEQ ID NO: 62 - protein, 118 aa, synthetic construct
QSVEESGGRLVTPGTPLTLTCTVSGFSLSTYAMNWVRQAPGKGLEWIGIIYSNGDGYYASWAKGRYTISKTSSTTVDLKVTSPTTEDTATYFCARGIYSQTTGYFNLWGQGTLVTVSS

SEQ ID NO: 63 - protein, 119 aa, synthetic construct
QSLEESGGRLVTPGTPLTLTCTASAFSLSGYYMGWVRQAPGKGLEYIGIIGGSSNTFYASWAKGRFTISKTPTTVDLKITSPTTEDTATYFCARHGDTDYISGTYFDLWGQGTLVTVSS

SEQ ID NO: 64 - protein, 119 aa, synthetic construct
QSLEESGGDLVKPGASLTLTCTASGFTLSSDYYMCWVRQAPGKGLELIACITTSTVGTWYASWAKGRFTISKTSSTTVTLQMTSLTAADTATYFCGREDGTYADYFNLWGPGTLVTVSS

SEQ ID NO: 65 - protein, 116 aa, synthetic construct
QSLEESGGRLVTPGTPLTLTCTASGFSLSSYWMSWVRQAPGKGLEWIGDIYASGSEYYASWVNGRFTGSKTSTTVDLKITSPTIEDTATYFCARHSSRSGIDFDLWGQGTLVTVSS

SEQ ID NO: 66 - protein, 119 aa, synthetic construct; identical to SEQ ID NO: 63

SEQ ID NO: 67 - protein, 117 aa, synthetic construct
QSVEESGGRLVTPGTPLTLTCTVSGFSLNSYAMGWVRQAPGKGLEYIGIINSSGSTWYATWVKGRFTISKTSTTVDLKMTSVTTEDTATYFCARNRDPFYITDMDLWGQGTLVTVSS

SEQ ID NO: 68 - protein, 114 aa, synthetic construct
QSLEESGGRLVTPGTPLTLTCTASGFSLSNSYMGWVRQAPGKGLEYIGFIDTGGTTYYASWAKGRFTISRTSTTVDLKMTSPTTEDTATYFCAKGPDDYFFNLWGQGTLVTVSS

SEQ ID NO: 69 - protein, 116 aa, synthetic construct
QEQLKESGGGLVTPGGTLTLTCTASGFTISGYYMNWVRQAPGKGLEWIGFIYSPSTTYYANWAKGRFTISKTSTTVDLKITSPTTEDTATYFCARGHTTEIRLDLWGQGTLVTVSS

SEQ ID NO: 70 - protein, 119 aa, synthetic construct
QSLEESGGRLVTPGGSLTLTCTVSGFSLSSYAMIWVRQAPGEGLEWIGTIDPSDNTDYASWAKGRFTISKTSSTTVDLKLTSLTTEDTATYFCASHSYDDYGRDVWNLWGQGTLVTVSS

SEQ ID NO: 71 - protein, 110 aa, synthetic construct
AYDMTQTPASVEAAVGGTVTIKCQASQSIGNYLAWYQQKPGQPPKQLIYRASTLASGVPSRFKGSGSGTEFTLTISDLECADGATYYCQQGYDSNNVDNFFGGGTEVVVR

SEQ ID NO: 72 - protein, 112 aa, synthetic construct
ADIVMTQTPASVEAAVGGIVIIKCQASESIGNTLAWYQQKPGQPPKLLIYGASTLASGVSSRFKGSGSGTEVTLTISDLECADAATYYCQSYWSSTTTFRDTFGGGTEVVVK

SEQ ID NO: 73 - protein, 110 aa, synthetic construct
AYHMTQTPASVEVPVGGTVTIKCQASQSISIYLAWYQQKPGQPPKLLIYRASTLASGVPSRFIGSGSGTEFSLTISDLECADAATYYCQQGYEDSNVESLFGGGTEVVVK

SEQ ID NO: 74 - protein, 110 aa, synthetic construct
AYDMTQTPASVEVAVGGTVTIKCQASQSISSWLAWYQQKPGQRPKLLIYRASTLASGVSSRFKGSGSGTQFTLTISGVECADAATYYCQQGYRSSNLDNAFGGGTEVVVK

SEQ ID NO: 75 - protein, 110 aa, synthetic construct
AYDMTQTPASVEAAVGGTVTIKCQASQSISNYLAWYQQKPGQPPKQLIYRASTLASGVPSRFKGSGSGTEFTLTMSDLECADAATYYCQQGYDSNNVDNLFGGGTEVVVK

SEQ ID NO: 76 - protein, 110 aa, synthetic construct
AYHMTQTPASVEVPVGGTVTIKCQASQSISIYLAWYQQKPGQPPKLLIYKASTLASGVPSRFIGSGSGTEFSLTISDLECADAATYYCQQGYEDSNVESLFGGGTEVVVK

SEQ ID NO: 77 - protein, 109 aa, synthetic construct
QVLTQTPSSVSAAVGGTVTINCQASQNIYSYLSWYQQKPGQPPKLLIYRASTLASGVPSRFKGSGSGTEFTLTISGVECADAATYYCQQGDSYSDVDNIFGGGTEVVVK

SEQ ID NO: 78 - protein, 111 aa, synthetic construct
ADIVMTQTPASVEAAVGGTITINCQASESISSWLAWYQQKPGQPPKLLIYKASTLESGVPSRFKGSGSGTEFTLTISGVQCADAATYYCQVYYFSYNSYNFFGGGTEVVVK

SEQ ID NO: 79 - protein, 110 aa, synthetic construct
AVLTQTPSPVSAAVGGTVSISCQSSESVYKNNRLSWFQQKPGQPPKLLIFDASTLASGVSSRFKGSGSGTQFTLTISGVQCDDAATYYCLGEFRDSSDNSFGGGTEVVVK

SEQ ID NO: 80 - protein, 111 aa, synthetic construct
IVMTQTPSSKSVPVGDTVTINCQASQSVDNNNDLSWYQQKPGQPPKLLIYYTSSLPSGVPSRFKGSGSGTQFTLTISDLECDDAATYYCVGYKSSTTDATAFGGGTEVMVK

SEQ ID NO: 81 - DNA, 15 bp, synthetic construct
  source 1..15: mol_type = other DNA
agctactggatgagc

SEQ ID NO: 82 - DNA, 15 bp, synthetic construct
  source 1..15: mol_type = other DNA
acctatgcgatgaac

SEQ ID NO: 83 - DNA, 15 bp, synthetic construct
  source 1..15: mol_type = other DNA
ggctactacatgggc

SEQ ID NO: 84 - DNA, 18 bp, synthetic construct
  source 1..18: mol_type = other DNA
agcgactactacatgtgc

SEQ ID NO: 85 - DNA, 15 bp, synthetic construct
  source 1..15: mol_type = other DNA
agttactggatgagc

SEQ ID NO: 86 - DNA, 15 bp, synthetic construct
  source 1..15: mol_type = other DNA
ggctactacatgagc

SEQ ID NO: 87 - DNA, 15 bp, synthetic construct
  source 1..15: mol_type = other DNA
agctatgcaatgggc

SEQ ID NO: 88 - DNA, 15 bp, synthetic construct
  source 1..15: mol_type = other DNA
aactcgtacatgggc

SEQ ID NO: 89 - DNA, 15 bp, synthetic construct
  source 1..15: mol_type = other DNA
ggctactacatgaac

SEQ ID NO: 90 - DNA, 15 bp, synthetic construct
  source 1..15: mol_type = other DNA
agctatgcaatgatc

SEQ ID NO: 91 - DNA, 48 bp, synthetic construct
  source 1..48: mol_type = other DNA
gacatttatgctactggtagcacatactacgcgaactgggcgacaggc

SEQ ID NO: 92 - DNA, 48 bp, synthetic construct
  source 1..48: mol_type = other DNA
atcatttatagtaatggtgatggatattacgcgagctgggcgaaaggc

SEQ ID NO: 93 - DNA, 48 bp, synthetic construct
  source 1..48: mol_type = other DNA
atcattggtggtagtagtaacacattctacgcgagctgggcgaaaggc

SEQ ID NO: 94 - DNA, 51 bp, synthetic construct
  source 1..51: mol_type = other DNA
tgcattactactagtactgttggcacatggtacgcgagctgggcgaaaggc

SEQ ID NO: 95 - DNA, 48 bp, synthetic construct
  source 1..48: mol_type = other DNA
gacatttatgctagtggtagtgaatactacgcgagctgggtgaatggc

SEQ ID NO: 96 - DNA, 48 bp, synthetic construct
  source 1..48: mol_type = other DNA
atcattggtggtagtggtaacacattctacgcgagctgggcgaaaggc

SEQ ID NO: 97 - DNA, 48 bp, synthetic construct
  source 1..48: mol_type = other DNA
atcattaatagtagtggtagcacatggtacgcgacctgggtgaaaggc

SEQ ID NO: 98 - DNA, 48 bp, synthetic construct
  source 1..48: mol_type = other DNA
tttattgacactggtggtaccacatactacgcgagctgggcgaaaggc

SEQ ID NO: 99 - DNA, 48 bp, synthetic construct
  source 1..48: mol_type = other DNA
ttcatttatagtcctagtaccacatactacgcgaactgggcgaaaggc

SEQ ID NO: 100 - DNA, 48 bp, synthetic construct
  source 1..48: mol_type = other DNA
accattgatcctagtgataacaccgactacgcgagttgggcaaaaggc

SEQ ID NO: 101 - DNA, 33 bp, synthetic construct
  source 1..33: mol_type = other DNA
caggccagtcagagcattggtaactacttagcc

SEQ ID NO: 102 - DNA, 36 bp, synthetic construct
  source 1..36: mol_type = other DNA
ggcatttattctcagactactggctactttaacttg

SEQ ID NO: 103 - DNA, 42 bp, synthetic construct
  source 1..42: mol_type = other DNA
catggggataccgattatatttctggtacctactttgacttg

SEQ ID NO: 104 - DNA, 33 bp, synthetic construct
  source 1..33: mol_type = other DNA
gaagatggtacttatgctgattatttcaacttg

SEQ ID NO: 105 - DNA, 33 bp, synthetic construct
  source 1..33: mol_type = other DNA
catagtagtaggagtggtattgattttgacttg

SEQ ID NO: 106 - DNA, 42 bp, synthetic construct; identical to SEQ ID NO: 103
  source 1..42: mol_type = other DNA

SEQ ID NO: 107 - DNA, 36 bp, synthetic construct
  source 1..36: mol_type = other DNA
aatagagatcctttttacattactgatatggacttg

SEQ ID NO: 108 - DNA, 27 bp, synthetic construct
  source 1..27: mol_type = other DNA
gggcctgatgattatttctttaatttg

SEQ ID NO: 109 - DNA, 30 bp, synthetic construct
  source 1..30: mol_type = other DNA
ggtcatactactgaaattcggttggatctc

SEQ ID NO: 110 - DNA, 39 bp, synthetic construct
  source 1..39: mol_type = other DNA
cattcctacgatgactatggtagggacgtctggaacttg

SEQ ID NO: 111 - DNA, 33 bp, synthetic construct; identical to SEQ ID NO: 101
  source 1..33: mol_type = other DNA

SEQ ID NO: 112 - DNA, 33 bp, synthetic construct
  source 1..33: mol_type = other DNA
caggccagtgagagcattggcaatacattagcc

SEQ ID NO: 113 - DNA, 33 bp, synthetic construct
  source 1..33: mol_type = other DNA
caggccagtcagagcattagtatctacttagcc

SEQ ID NO: 114 - DNA, 33 bp, synthetic construct
  source 1..33: mol_type = other DNA
caggccagtcagagcattagcagttggttagcc

SEQ ID NO: 115 - DNA, 33 bp, synthetic construct
  source 1..33: mol_type = other DNA
caggccagtcagagcattagtaactacttagcc

SEQ ID NO: 116 - DNA, 33 bp, synthetic construct; identical to SEQ ID NO: 113
  source 1..33: mol_type = other DNA

SEQ ID NO: 117 - DNA, 33 bp, synthetic construct
  source 1..33: mol_type = other DNA
caggccagtcagaacatttacagctacttatcc

SEQ ID NO: 118 - DNA, 33 bp, synthetic construct
  source 1..33: mol_type = other DNA
caagccagtgagagcattagcagttggttagcc

SEQ ID NO: 119 - DNA, 39 bp, synthetic construct
  source 1..39: mol_type = other DNA
cagtccagtgagagtgtttataagaacaaccgcttatcc

SEQ ID NO: 120 - DNA, 39 bp, synthetic construct
  source 1..39: mol_type = other DNA
caggccagtcagagtgttgataataacaacgacttatcc

SEQ ID NO: 121 - DNA, 21 bp, synthetic construct
  source 1..21: mol_type = other DNA
agggcatccactctggcatct

SEQ ID NO: 122 - DNA, 21 bp, synthetic construct
  source 1..21: mol_type = other DNA
ggtgcatccactctggcatct

SEQ ID NO: 123 - DNA, 21 bp, synthetic construct
  source 1..21: mol_type = other DNA
agggcatccactctggcatct

SEQ ID NO: 124 - DNA, 21 bp, synthetic construct
  source 1..21: mol_type = other DNA
agggcatccactctggcatct

SEQ ID NO: 125 - DNA, 21 bp, synthetic construct
  source 1..21: mol_type = other DNA
agggcatccactctggcatct

SEQ ID NO: 126 - DNA, 21 bp, synthetic construct
  source 1..21: mol_type = other DNA
aaggcttccactctggcatct

SEQ ID NO: 127 - DNA, 21 bp, synthetic construct
  source 1..21: mol_type = other DNA
agggcatccactctggcatct

SEQ ID NO: 128 - DNA, 21 bp, synthetic construct
  source 1..21: mol_type = other DNA
aaggcatccactctggaatct

SEQ ID NO: 129 - DNA, 21 bp, synthetic construct
  source 1..21: mol_type = other DNA
gatgcatccactctggcatct

SEQ ID NO: 130 - DNA, 21 bp, synthetic construct
  source 1..21: mol_type = other DNA
tatacatccagcctgccatct

SEQ ID NO: 131 - DNA, 36 bp, synthetic construct
  source 1..36: mol_type = other DNA
caacagggttatgatagtaataatgttgataatttt

SEQ ID NO: 132 - DNA, 39 bp, synthetic construct
  source 1..39: mol_type = other DNA
caaagttattggagtagtactacaactttcagggatact

SEQ ID NO: 133 - DNA, 36 bp, synthetic construct
  source 1..36: mol_type = other DNA
caacagggttatgaggatagtaatgttgagagtctt

SEQ ID NO: 134 - DNA, 36 bp, synthetic construct
  source 1..36: mol_type = other DNA
caacagggttatcgtagtagtaatttagataatgct

SEQ ID NO: 135 - DNA, 36 bp, synthetic construct
  source 1..36: mol_type = other DNA
caacagggttatgatagtaataatgttgataatctt

SEQ ID NO: 136 - DNA, 36 bp, synthetic construct; identical to SEQ ID NO: 133
  source 1..36: mol_type = other DNA

SEQ ID NO: 137 - DNA, 36 bp, synthetic construct
  source 1..36: mol_type = other DNA
caacagggtgatagttatagtgatgttgataatatt

SEQ ID NO: 138 - DNA, 36 bp, synthetic construct
  source 1..36: mol_type = other DNA
caggtctattattttagttataatagttacaatttt

SEQ ID NO: 139 - DNA, 33 bp, synthetic construct
  source 1..33: mol_type = other DNA
ctaggcgaatttagggatagtagtgataatagt

SEQ ID NO: 140 - DNA, 36 bp, synthetic construct
  source 1..36: mol_type = other DNA
gtagggtataaaagtagtactactgatgctactgct

SEQ ID NO: 141 - DNA, 348 bp, synthetic construct
  source 1..348: mol_type = other DNA
cagtcgctggaggagtccgggggtcgcctggtcacgcctgggacacccctgacactcacctgcacagcctctggattctccctcagtagctactggatgagctgggtccgccaggctccagggaaggggctggaatggatcggagacatttatgctactggtagcacatactacgcgaactgggcgacaggccgattcaccggctccaaaacctcgaccacggtggatctgacaatcaccagtccgacaatcgaggacacggccacctatttctgtgccagacatagtagtaggagtggtattgattttgacttgtggggccaaggcaccctggtcaccgtctcctca

SEQ ID NO: 142 - DNA, 354 bp, synthetic construct
  source 1..354: mol_type = other DNA
cagtcggtggaggagtccgggggtcgcctggtcacgcctgggacacccctgacactcacctgcacagtctctggattctccctcagtacctatgcgatgaactgggtccgccaggctccagggaaggggctggaatggatcggaatcatttatagtaatggtgatggatattacgcgagctgggcgaaaggccgatataccatctccaaaacctcgtcgaccacggtggatctgaaagtcaccagtccgacaaccgaggacacggccacctatttctgtgccagaggcatttattctcagactactggctactttaacttgtggggccaaggcaccctggtcaccgtctcatca

SEQ ID NO: 143 - DNA, 357 bp, synthetic construct
  source 1..357: mol_type = other DNA
cagtcgctggaggagtccgggggtcgcctggtcacgcctgggacacccctgacactcacctgcacagcctctgcattctccctcagtggctactacatgggctgggtccgccaggctccagggaaggggctggaatacatcggaatcattggtggtagtagtaacacattctacgcgagctgggcgaaaggccgattcaccatctccaaaaccccgaccacggtggatctgaaaatcaccagtccgacaaccgaggacacggccacctatttctgtgccagacatggggataccgattatatttctggtacctactttgacttgtggggccaaggcaccctggtcaccgtctcctca

SEQ ID NO: 144 - DNA, 357 bp, synthetic construct
  source 1..357: mol_type = other DNA
cagtcgttggaggagtccgggggagacctggtcaagcctggggcatccctgacactcacctgcacagcctctggattcaccctcagtagcgactactacatgtgctgggtccgccaggctccagggaaggggctggaattgatcgcatgcattactactagtactgttggcacatggtacgcgagctgggcgaaaggccgattcaccatctccaaaacctcgtcgaccacggtgactctgcaaatgaccagtctgacagccgcggacacggccacctatttctgtgggagagaagatggtacttatgctgattatttcaacttgtggggcccaggcaccctggtcaccgtctcctca

SEQ ID NO: 145 - DNA, 348 bp, synthetic construct
  source 1..348: mol_type = other DNA
cagtcgctggaggagtccgggggtcgcctggtcacgcctgggacacccctgacactcacctgcacagcctctggattctccctcagtagttactggatgagctgggtccgccaggctccagggaaggggctggaatggatcggagacatttatgctagtggtagtgaatactacgcgagctgggtgaatggccgattcaccggctccaaaacctcgaccacggtggatctgaaaatcaccagtccgacaatcgaggacacggccacctatttctgtgccagacatagtagtaggagtggtattgattttgacttgtggggccaaggcaccctggtcaccgtctcctca

SEQ ID NO: 146 - DNA, 357 bp, synthetic construct
  source 1..357: mol_type = other DNA
cagtcgctggaggagtccgggggtcgcctggtcacgcctgggacacccctgacactcacctgcacagcctctgggttctccctcagtggctactacatgagctgggtccgccaggctccagggaaggggctggaatacatcggaatcattggtggtagtggtaacacattctacgcgagctgggcgaaaggccgattcaccatctccaaaaccccgaccacggtggatctgaaaatcaccagtccgacaaccgaggacacggccacctatttctgtgccagacatggggataccgattatatttctggtacctactttgacttgtggggccaaggcaccctggtcaccgtctcctca

SEQ ID NO: 147 - DNA, 351 bp, synthetic construct
  source 1..351: mol_type = other DNA
cagtcggtggaggagtccgggggtcgcctggtcacgcctgggacacccctgacactcacctgcacagtctctggattctccctcaatagctatgcaatgggctgggtccgccaggctccagggaaggggctggaatacatcggaatcattaatagtagtggtagcacatggtacgcgacctgggtgaaaggccgattcaccatctccaaaacctcgaccacggtggatctgaaaatgaccagtgtgacaaccgaggacacggccacctatttctgtgccagaaatagagatcctttttacattactgatatggacttgtggggccaaggcaccctggtcaccgtctcctca

SEQ ID NO: 148 - DNA, 342 bp, synthetic construct
  source 1..342: mol_type = other DNA
cagtcgctggaggagtccgggggtcgcctggtcacgcctgggacacccctgacactcacctgcacagcctctggattctccctcagtaactcgtacatgggctgggtccgccaggctccagggaaggggctggaatacatcggatttattgacactggtggtaccacatactacgcgagctgggcgaaaggccgattcaccatctccagaacctcgaccacggtggatctgaaaatgaccagtccgacaaccgaggacacggccacctatttctgtgccaaagggcctgatgattatttctttaatttgtggggccaaggcaccctggtcaccgtctcctca

SEQ ID NO: 149 - DNA, 348 bp, synthetic construct
  source 1..348: mol_type = other DNA
caggagcagctgaaggagtccggaggaggcctggtaacgcctggaggaaccctgacactcacctgcacagcctctggattcaccatcagtggctactacatgaactgggtccgccaggctccagggaaggggctggaatggatcggattcatttatagtcctagtaccacatactacgcgaactgggcgaaaggccgattcaccatctccaaaacctcgaccaccgtggatctgaaaatcaccagtccgacaaccgaggacacggccacctatttttgtgccagaggtcatactactgaaattcggttggatctctggggccagggcaccctggtcaccgtctcctca

SEQ ID NO: 150 - DNA, 357 bp, synthetic construct
  source 1..357: mol_type = other DNA
cagtcgctggaggagtccgggggtcgcctggtaacgcctggaggatccctgacactcacctgcacagtctctggattctccctcagtagctatgcaatgatctgggtccgccaggctccaggggaggggctggaatggatcggaaccattgatcctagtgataacaccgactacgcgagttgggcaaaaggccgattcaccatctccaaaacctcgtcgaccacggtggatctgaaattgaccagtctgacaaccgaggacacggccacctatttctgtgccagtcattcctacgatgactatggtagggacgtctggaacttgtggggccaaggcaccctggtcaccgtctcctca

SEQ ID NO: 151 - DNA, 330 bp, synthetic construct
  source 1..330: mol_type = other DNA
gcctatgatatgacccagactccagcctccgtggaggcagctgtgggaggcacagtcaccatcaagtgccaggccagtcagagcattggtaactacttagcctggtatcagcagaaaccagggcagcctcccaagcaactgatctacagggcatccactctggcatctggggtcccatcgcggttcaaaggcagtggatctgggacagagttcactctcaccatcagcgacctggagtgtgccgatggtgccacttactactgtcaacagggttatgatagtaataatgttgataattttttcggcggagggaccgaggtggtggtcaga

SEQ ID NO: 152 - DNA, 336 bp, synthetic construct
  source 1..336: mol_type = other DNA
gctgacattgtgatgacccagactccagcctccgtggaggcagctgtgggaggcatagtcatcatcaagtgccaggccagtgagagcattggcaatacattagcctggtatcagcagaaaccagggcagcctcccaagctcctgatttatggtgcatccactctggcatctggggtctcatcgcggttcaaaggcagtggatctgggacagaggtcactctcaccatcagcgacctggagtgtgccgatgctgccacttactattgtcaaagttattggagtagtactacaactttcagggatactttcggcggagggaccgaggtggtggtcaaa

SEQ ID NO: 153 - DNA, 330 bp, synthetic construct
  source 1..330: mol_type = other DNA
gcctatcatatgacccagactccagcctctgtggaggtgcctgtgggaggcacagtcaccatcaagtgccaggccagtcagagcattagtatctacttagcctggtatcagcagaaaccagggcagcctcccaagctcctgatctacagggcatccactctggcatctggggtcccatcgcggttcataggcagtggatctgggacagagttcagtctcaccatcagcgacctggagtgtgccgatgctgccacttactactgtcaacagggttatgaggatagtaatgttgagagtcttttcggcggagggaccgaggtggtggtcaaa

SEQ ID NO: 154 - DNA, 330 bp, synthetic construct
  source 1..330: mol_type = other DNA
gcctatgatatgacccagactccagcctctgtggaggtagctgtgggaggcacagtcaccatcaagtgccaggccagtcagagcattagcagttggttagcctggtatcagcagaaaccagggcagcgtcccaagctcctgatctacagggcatccactctggcatctggggtctcatcgcgattcaaaggcagtggatctgggacacagttcactctcaccatcagcggcgtggagtgtgccgatgctgccacttactactgtcaacagggttatcgtagtagtaatttagataatgctttcggcggagggaccgaggtggtggtcaaa

SEQ ID NO: 155 - DNA, 330 bp, synthetic construct
  source 1..330: mol_type = other DNA
gcctatgatatgacccagactccagcctccgtggaggcagctgtgggaggcacagtcaccatcaagtgccaggccagtcagagcattagtaactacttagcctggtatcagcagaaaccagggcagccccccaagcaactgatctacagggcatccactctggcatctggggtcccatcgcggttcaaaggcagtggatctgggacagagttcactctcaccatgagcgacctggagtgtgccgatgctgccacttactactgtcaacagggttatgatagtaataatgttgataatcttttcggcggagggaccgaggtggtggtcaaa

SEQ ID NO: 156 - DNA, 330 bp, synthetic construct
  source 1..330: mol_type = other DNA
gcctatcatatgacccagactccagcctctgtggaggtgcctgtgggaggcacagtcaccatcaagtgccaggccagtcagagcattagtatctacttagcctggtatcagcagaaaccagggcagcctcccaagctcctgatctacaaggcttccactctggcatctggggtcccatcgcggttcataggcagtggatctgggacagagttcagtctcaccatcagcgacctggagtgtgccgatgctgccacttactactgtcaacagggttatgaggatagtaatgttgagagtcttttcggcggagggaccgaggtggtggtcaaa

SEQ ID NO: 157 - DNA, 327 bp, synthetic construct
  source 1..327: mol_type = other DNA
caagtgctgacccagactccatcctccgtgtctgcagctgtgggaggcacagtcaccatcaattgccaggccagtcagaacatttacagctacttatcctggtatcagcagaaaccagggcagcctcccaagctcctgatctacagggcatccactctggcatctggggtcccatcgcggttcaaaggcagtggatctgggacagagttcactctcaccatcagcggcgtggagtgtgccgatgctgccacttactactgtcaacagggtgatagttatagtgatgttgataatattttcggcggagggaccgaggtggtggtcaaa

SEQ ID NO: 158 - DNA, 333 bp, synthetic construct
  source 1..333: mol_type = other DNA
gctgacattgtgatgacccagactccagcctccgtggaggcagctgtgggaggcacaatcaccatcaattgccaagccagtgagagcattagcagttggttagcctggtatcagcagaaaccagggcagcctcccaagctcctgatctacaaggcatccactctggaatctggggtcccatcgcggttcaaaggcagtggatctgggacagagttcactctcaccatcagcggcgtgcagtgtgccgatgctgccacttactactgtcaggtctattattttagttataatagttacaattttttcggcggagggaccgaggtggtggtcaag

SEQ ID NO: 159 - DNA, 330 bp, synthetic construct
  source 1..330: mol_type = other DNA
gccgtgctgacccagactccatctcccgtgtctgcagctgtgggaggcacagtcagcatcagttgccagtccagtgagagtgtttataagaacaaccgcttatcctggtttcagcagaaaccagggcagcctcccaaactcctgatctttgatgcatccactctggcatctggggtctcatcgcgattcaaaggcagtggatctgggacacagttcactctcaccatcagtggcgtgcagtgtgacgatgctgccacttactactgtctaggcgaatttagggatagtagtgataatagtttcggcggagggaccgaggtggtggtcaaa

SEQ ID NO: 160 - DNA, 333 bp, synthetic construct
  source 1..333: mol_type = other DNA
atcgtgatgacccagactccatcttccaagtctgtccctgtgggagacacagtcaccatcaattgccaggccagtcagagtgttgataataacaacgacttatcctggtatcagcagaaaccagggcagcctcccaagctcctgatctattatacatccagcctgccatctggggtcccatcgcggttcaaaggcagtggatctgggacacagttcactctcaccatcagcgacctggagtgtgacgatgctgccacttactactgtgtagggtataaaagtagtactactgatgctactgctttcggcggagggaccgaggtgatggtcaaa

SEQ ID NO: 161 - protein, 473 aa, synthetic construct
MESVLFWVFLVTILKGVQGEVRLVESGGTLVKPGGSLKLSCVASGFTFRRYSMDWVRQAPGKSLQWVAGINGDGTGTSYSQTVKGRFTISRDNAKNTLYLQINSLRAEDSAVYYCAKSWSRNGDLDYWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPGK

SEQ ID NO: 162 - DNA, 1453 bp, synthetic construct
  source 1..1453: mol_type = other DNA
agtgctcaggacaccacacagacaaatcaccatggagtctgtgctcttctgggttttccttgtcactattttaaaaggtgtccagggtgaggtacgtttggtggagtctggaggaaccctggtgaagcctggggggtccctgaaactctcttgtgtggcctctggattcaccttcagaagatactccatggactgggtccgccaggctccaggcaagagcctgcagtgggtcgccgggattaacggtgatggcacaggaacatcctattcacagactgtgaagggccgattcaccatctccagagacaacgccaagaacaccctctatctgcagataaacagcctgagagccgaagactctgctgtgtattattgtgccaagagctggtctcgtaatggggatcttgactactggggccagggaaccctggtcaccgtctcctcagcctccaccacggccccctcggttttcccactggcccccagctgcgggtccacttccggctccacggtggccctggcctgcctggtgtcaggctacttccccgagcctgtaactgtgtcctggaattccggctccttgaccagcggtgtgcacaccttcccgtccgtcctgcagtcctcagggctctactccctcagcagcatggtgacagtgccctccagcaggtggcccagcgagaccttcacctgcaacgtggcccacccggccagcaaaactaaagtagacaagccagtgcccaaaagagaaaatggaagagttcctcgcccacctgattgtcccaaatgcccagcccctgaaatgctgggagggccttcggtcttcatctttcccccgaaacccaaggacaccctcttgattgcccgaacacctgaggtcacatgtgtggtggtggatctggacccagaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaacaacaaagccctcccatccccgatcgagaggaccatctccaaggccagagggcaagcccatcagcccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacaggaatccctctcccattctccgggtaaatga

SEQ ID NO: 163 - protein, 453 aa, synthetic construct
QSLEESGGRLVTPGTPLTLTCTASAFSLSGYYMGWVRQAPGKGLEYIGIIGGSSNTFYASWAKGRFTISKTPTTVDLKITSPTTEDTATYFCARHGDTDYISGTYFDLWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 164 - protein, 450 aa, synthetic construct
QSLEESGGRLVTPGTPLTLTCTASGFSLSSYWMSWVRQAPGKGLEWIGDIYATGSTYYANWATGRFTGSKTSTTVDLTITSPTIEDTATYFCARHSSRSGIDFDLWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 165 - protein, 452 aa, synthetic construct
QSVEESGGRLVTPGTPLTLTCTVSGFSLSTYAMNWVRQAPGKGLEWIGIIYSNGDGYYASWAKGRYTISKTSSTTVDLKVTSPTTEDTATYFCARGIYSQTTGYFNLWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 166 - protein, 216 aa, synthetic construct
AYHMTQTPASVEVPVGGTVTIKCQASQSISIYLAWYQQKPGQPPKLLIYRASTLASGVPSRFIGSGSGTEFSLTISDLECADAATYYCQQGYEDSNVESLFGGGTEVVVKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSEC

SEQ ID NO: 167 - protein, 216 aa, synthetic construct
AYDMTQTPASVEAAVGGTVTIKCQASQSIGNYLAWYQQKPGQPPKQLIYRASTLASGVPSRFKGSGSGTEFTLTISDLECADGATYYCQQGYDSNNVDNFFGGGTEVVVRRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSEC

SEQ ID NO: 168 - protein, 218 aa, synthetic construct
ADIVMTQTPASVEAAVGGIVIIKCQASESIGNTLAWYQQKPGQPPKLLIYGASTLASGVSSRFKGSGSGTEVTLTISDLECADAATYYCQSYWSSTTTFRDTFGGGTEVVVKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSEC

SEQ ID NO: 169 - DNA, 1353 bp, synthetic construct
  source 1..1353: mol_type = other DNA
caaagtctagaagaaagtggtggtcgactagtcacacccggaacccctctgacactgacctgtaccgcctctggcttttctctgtcctcctactggatgtcctgggtgcggcaggctcctggcaagggcctggaatggatcggcgacatctacgctaccggctccacctactacgccaactgggccaccggcagattcaccggatccaagacctctaccaccgtggatctcacaatcacctctcctaccatcgaggacaccgctacatatttctgcgccagacactctagccggtccggcatcgacttcgacctgtggggccagggcaccctggtgaccgtgtccagcgcttccaccacagctccttctgtgttccctctggctccctcctgcggctctacctccggatccacagtggctctggcctgcctggtttccggctactttcccgaacctgtcaccgtctcttggaactccggctccctgacctctggcgtgcacaccttccctagcgtgctgcagagctctggcctgtactccctgtcctccatggtgaccgtgccaagctctcggtggccttccgagacatttacatgcaacgtggcccaccctgcttcgaaaacaaaagtggacaagcctgtgccaaaacgggagaatggcagagtgcctcggcctcccgactgtcctaagtgccctgcccctgagatgctgggcgggccttctgtgttcatcttcccacctaagcccaaggataccctgctgatcgccagaactcctgaggtgacctgtgtggtggtcgacctggatcctgaggaccccgaagtgcagatctcttggttcgtggacggcaaacagatgcagaccgccaagacccagcctagagaggaacagttcaacggcacctaccgcgtcgtgtccgtgctgcctatcggccaccaggactggctgaagggcaagcagtttacctgcaaggtgaacaacaaggctctgccttctcccatcgagcggaccatctccaaggcccggggacaggcccatcaacccagcgtgtacgtgttgcccccaagcagagaagagctctctaagaacaccgtgagcctgacctgtctgatcaaggacttcttccctcctgacatcgacgtggaatggcagtccaacggccagcaggaacctgagtccaagtacagaaccacccctcctcagctggacgaggatggctcctacttcctgtattccaagctgtccgtggataagtccagatggcagagaggcgacaccttcatttgcgccgtgatgcacgaggccctgcacaaccactacacccaagagtctctgtctcactctcctggctga

SEQ ID NO: 170 - DNA, 1353 bp, synthetic construct; identical to SEQ ID NO: 169
  source 1..1353: mol_type = other DNA

SEQ ID NO: 171 - DNA, 1359 bp, synthetic construct
  source 1..1359: mol_type = other DNA
cagtctgtggaagaatccggcggcagactggtgacacctggcacccctctgaccctaacctgcacagtgtctggcttcagcctcagcacctatgccatgaactgggtgcggcaagctcctggcaagggcctggaatggatcggcatcatctactccaacggcgacggctactacgcctcctgggccaagggcagatacacaatttccaaaaccagctccacaaccgtggatctgaaggtgacctctcctaccaccgaagataccgctacctacttctgtgctagaggcatctacagtcagaccaccggctatttcaacctgtggggccaaggcaccctggtcaccgtgtcttccgcctccaccaccgccccttccgtgtttcctctggctcctagctgcggctctacctccggctctaccgtggctctggcctgcctggtatctggctacttccctgagcctgtgaccgtgtcttggaactccggaagcctgacctcaggagtgcacacctttccatctgtcctgcagtcctctggcctgtactctctgagctccatggtgactgtgccttcatccagatggccttccgagaccttcacatgcaacgtggcccatcctgcttccaagaccaaggtcgacaagcctgtgccaaagagagagaacggaagagtgccccggcctcctgactgccccaagtgccctgctccagagatgctgggcggccctagcgtgttcatcttcccacctaagcccaaggacaccctgctgatcgccagaacccctgaagtgacatgtgtggtggtggacctggatcccgaggaccctgaggtgcagatctcctggttcgtggacggcaagcagatgcagaccgctaaaacacagcctcgcgaggagcagtttaatggtacctacagagtggtctctgtgctgcctatcggccaccaggactggctgaaaggaaaacagttcacctgcaaggtgaacaacaaggccctgccttctcccatcgagcggaccatctccaaggctagaggccaggcccatcagccttctgtgtacgtgctgcccccatcccgggaagagctgtccaagaacaccgtgtccctcacctgtctgatcaaggacttcttcccccccgacatcgacgtcgaatggcagtctaatggccagcaggaacccgagtccaagtaccggaccacccctcctcagctggacgaggatggctcctacttcctgtactccaagctgtctgtggacaagtccaggtggcagcggggcgatacattcatctgcgccgtgatgcacgaggccctgcacaaccactacactcaagagtccctgtctcactcccctggatga

SEQ ID NO: 172 - DNA, 651 bp, synthetic construct
  source 1..651: mol_type = other DNA
gcatatgatatgactcaaactccagcaagtgtcgaggccgccgtgggcggcaccgtgaccatcaagtgccaggcctctcagtccatcggcaactacctggcctggtatcagcagaagcctggccagcctcctaagcagctgatctacagagccagcacactggcttctggcgtgcccagcagatttaaaggctctggctccggcacagagttcaccctgaccatctccgacctggaatgtgctgatggcgctacctactactgccaacagggctacgactccaacaatgtggacaacttcttcggcggaggaaccgaggtggtggtgcggcggaacgacgcccagcctgccgtgtacctgttccagccctctccagatcagctgcacacaggatctgcttctgtggtgtgcctgctgaactccttctaccctaaagacatcaacgtgaagtggaaggtggacggcgtgatccaggataccggcatccaagagtccgtgaccgaacaggacaaggactccacctactctctgtccagcacactgaccatgtccagcaccgagtatctcagccatgaactgtactcctgcgagatcacccacaagtccctgccttctaccctgatcaagtcttttcagagatccgagtgttga

SEQ ID NO: 173 - DNA, 651 bp, synthetic construct; identical to SEQ ID NO: 172
  source 1..651: mol_type = other DNA

SEQ ID NO: 174 - DNA, 657 bp, synthetic construct
  source 1..657: mol_type = other DNA
gccgacatcgtgatgacccagacccctgcctctgtggaagccgccgtgggcggcatcgtgatcatcaagtgccaggcctccgagtccatcggcaacaccctggcttggtatcagcagaagcccggtcagcctccaaagctgctgatctacggcgctagcaccctggcctctggcgtgtccagcagattcaagggctctggctccggcaccgaggtgacactgaccatctccgacctggaatgtgccgatgctgctacctactactgccagtcctactggtcctctaccaccacatttcgggacaccttcggcggaggaacagaggtcgtggtgaaacggaacgacgcccagcctgccgtgtacctgttccagccctctccagatcagctgcacacaggatctgcttctgtggtgtgcctgctgaactccttctaccctaaagacatcaacgtgaagtggaaggtggacggcgtgatccaggataccggcatccaagagtccgtgaccgaacaggacaaggactccacctactctctgtccagcacactgaccatgtccagcaccgagtatctcagccatgaactgtactcctgcgagatcacccacaagtccctgccttctaccctgatcaagtcttttcagagatccgagtgttga

SEQ ID NO: 175 - protein, 456 aa, synthetic construct
EVQLVESGGDLVKPGGSLRLSCVASAFSLSGYYMGWVRQAPGKGLEWISIIGGSSNTFYASWAKGRFTISKDNSKNTLYLQMNSLRAEDTAVYFCARHGDTDYISGTYFDLWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 176 - protein, 456 aa, synthetic construct; identical to SEQ ID NO: 175

SEQ ID NO: 177 - protein, 473 aa, synthetic construct
METGLRWLLLVAVLKGVQCEVQLVESGGDLVKPGGSLRLSCVASGFSLSTYAMNWVRQAPGKGLEWISIIYSNGDGYYASWAKGRFTISRDNAKNTLYLQMNSLRAEDTAVYYCARGIYSQTTGYFNLWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 178 - protein, 473 aa, synthetic construct
METGLRWLLLVAVLKGVQCEVQLVESGGDLVKPGGSLRLSCVASGFSLSTYAMNWVRQAPGKGLEWISIIYSNGDGYYASWAKGRFTISRDNSKNTLYLQMNSLRAEDTAVYYCARGIYSQTTGYFNLWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 179 - protein, 453 aa, synthetic construct
EVQLVESGGDLVKPGGSLRLSCVASGFTFSSYWMSWVRQAPGKGLQWLSDIYATGSTYYANWATGRFTGSRDNAKNTLYLQMNSLTIEDTATYFCARHSSRSGIDFDLWGQGTLVTVSSASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQESLSHSPG

SEQ ID NO: 180 - protein, 453 aa, synthetic construct; identical to SEQ ID NO: 179

SEQ ID NO: 181 - protein, 216 aa, synthetic construct
EIVMTQSPASLSLSQGEKVTITCQASQSISIYLAWYQQKPGQPPKLLIYRASTLASGVPSRFSGSGSGTDFSFTITSLQPEDVATYYCQQGYEDSNVESLFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSEC

SEQ ID NO: 182 - protein, 216 aa, synthetic construct
EIVMTQSPASLSLSQGEKVTITCQASQSISIYLAWYQQKPGQPPKLLIYRASTLASGVPSRFSGSGSGTDFSFTISSLQPEDVATYYCQQGYEDSNVESLFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSEC

SEQ ID NO: 183 - protein, 239 aa, synthetic construct
MDTRAPTQLLGLLLLWLPGARCEIVMTQTPASLSLSQEGKVTITCQASESIGNTLAWYQQKPGQPPKLLIYGASTLASGVPSRFSGSGSGTDFTFTINSLQPEDVATYYCQSYWSSTTTFRDTFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSEC

SEQ ID NO: 184 - protein, 239 aa, synthetic construct; identical to SEQ ID NO: 183

SEQ ID NO: 185 - protein, 216 aa, synthetic construct
EIVMTQSPASLSLSQEEKVTITCQASQSIGNYLAWYQQKPGQAPKQLIYRASTLASGVPSRFKGSGSGTEFTLTISDLECADVAVYYCQQGYDSNNVDNFFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSEC

SEQ ID NO: 186 - protein, 216 aa, synthetic construct
EIVMTQSPASLSLSQEEKVTITCQASQSIGNYLAWYQQKPGQAPKQLIYRASTLASGVPSRFSGSGSGTDFTLTISDLECADVAVYYCQQGYDSNNVDNFFGQGTKVEIKRNDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSEC

SEQ ID NO: 187 - DNA, 1368 bp, synthetic construct
  source 1..1368: mol_type = other DNA
gaggtgcagctggtggagagcggcggcgacctggtgaagcccggcggcagcctgcggctgagctgcgtggccagcgccttcagcctgagcggctactacatgggctgggtgcggcaggcccccggcaagggcctggagtggatcagcatcatcggcggcagcagcaacaccttctacgccagctgggccaagggccggttcaccatcagcaaggacaacagcaagaacaccctgtacctgcagatgaacagcctgcgggccgaggacaccgccgtgtacttctgcgcccggcacggcgacaccgactacatcagcggcacctacttcgacctgtggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcatggtgaccgtgcccagcagccggtggcccagcgagaccttcacctgcaacgtggcccaccccgccagcaagaccaaggtggacaagcccgtgcccaagcgggagaacggccgggtgccccggccccccgactgccccaagtgccccgcccccgagatgctgggcggccccagcgtgttcatcttcccccccaagcccaaggacaccctgctgatcgcccggacccccgaggtgacctgcgtggtggtggacctggaccccgaggaccccgaggtgcagatcagctggttcgtggacggcaagcagatgcagaccgccaagacccagccccgggaggagcagttcaacggcacctaccgggtggtgagcgtgctgcccatcggccaccaggactggctgaagggcaagcagttcacctgcaaggtgaacaacaaggccctgcccagccccatcgagcggaccatcagcaaggcccggggccaggcccaccagcccagcgtgtacgtgctgccccccagccgggaggagctgagcaagaacaccgtgagcctgacctgcctgatcaaggacttcttcccccccgacatcgacgtggagtggcagagcaacggccagcaggagcccgagagcaagtaccggaccacccccccccagctggacgaggacggcagctacttcctgtacagcaagctgagcgtggacaagagccggtggcagcggggcgacaccttcatctgcgccgtgatgcacgaggccctgcacaaccactacacccaggagagcctgagccacagccccggc

SEQ ID NO: 188 - DNA, 1368 bp, synthetic construct; identical to SEQ ID NO: 187
  source 1..1368: mol_type = other DNA

SEQ ID NO: 189 - DNA, 1419 bp, synthetic construct
  source 1..1419: mol_type = other DNA
atggagaccggcctgcggtggctgctgctggtggccgtgctgaagggcgtgcagtgcgaggtgcagctggtggagagcggcggcgacctggtgaagcccggcggcagcctgcggctgagctgcgtggccagcggcttcagcctgagcacctacgccatgaactgggtgcggcaggcccccggcaagggcctggagtggatcagcatcatctacagcaacggcgacggctactacgccagctgggccaagggccggttcaccatcagccgggacaacgccaagaacaccctgtacctgcagatgaacagcctgcgggccgaggacaccgccgtgtactactgcgcccggggcatctacagccagaccaccggctacttcaacctgtggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcatggtgaccgtgcccagcagccggtggcccagcgagaccttcacctgcaacgtggcccaccccgccagcaagaccaaggtggacaagcccgtgcccaagcgggagaacggccgggtgccccggccccccgactgccccaagtgccccgcccccgagatgctgggcggccccagcgtgttcatcttcccccccaagcccaaggacaccctgctgatcgcccggacccccgaggtgacctgcgtggtggtggacctggaccccgaggaccccgaggtgcagatcagctggttcgtggacggcaagcagatgcagaccgccaagacccagccccgggaggagcagttcaacggcacctaccgggtggtgagcgtgctgcccatcggccaccaggactggctgaagggcaagcagttcacctgcaaggtgaacaacaaggccctgcccagccccatcgagcggaccatcagcaaggcccggggccaggcccaccagcccagcgtgtacgtgctgccccccagccgggaggagctgagcaagaacaccgtgagcctgacctgcctgatcaaggacttcttcccccccgacatcgacgtggagtggcagagcaacggccagcaggagcccgagagcaagtaccggaccacccccccccagctggacgaggacggcagctacttcctgtacagcaagctgagcgtggacaagagccggtggcagcggggcgacaccttcatctgcgccgtgatgcacgaggccctgcacaaccactacacccaggagagcctgagccacagccccggc

SEQ ID NO: 190 - DNA, 1419 bp, synthetic construct
  source 1..1419: mol_type = other DNA
atggagaccggcctgcggtggctgctgctggtggccgtgctgaagggcgtgcagtgcgaggtgcagctggtggagagcggcggcgacctggtgaagcccggcggcagcctgcggctgagctgcgtggccagcggcttcagcctgagcacctacgccatgaactgggtgcggcaggcccccggcaagggcctggagtggatcagcatcatctacagcaacggcgacggctactacgccagctgggccaagggccggttcaccatcagccgggacaacagcaagaacaccctgtacctgcagatgaacagcctgcgggccgaggacaccgccgtgtactactgcgcccggggcatctacagccagaccaccggctacttcaacctgtggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcatggtgaccgtgcccagcagccggtggcccagcgagaccttcacctgcaacgtggcccaccccgccagcaagaccaaggtggacaagcccgtgcccaagcgggagaacggccgggtgccccggccccccgactgccccaagtgccccgcccccgagatgctgggcggccccagcgtgttcatcttcccccccaagcccaaggacaccctgctgatcgcccggacccccgaggtgacctgcgtggtggtggacctggaccccgaggaccccgaggtgcagatcagctggttcgtggacggcaagcagatgcagaccgccaagacccagccccgggaggagcagttcaacggcacctaccgggtggtgagcgtgctgcccatcggccaccaggactggctgaagggcaagcagttcacctgcaaggtgaacaacaaggccctgcccagccccatcgagcggaccatcagcaaggcccggggccaggcccaccagcccagcgtgtacgtgctgccccccagccgggaggagctgagcaagaacaccgtgagcctgacctgcctgatcaaggacttcttcccccccgacatcgacgtggagtggcagagcaacggccagcaggagcccgagagcaagtaccggaccacccccccccagctggacgaggacggcagctacttcctgtacagcaagctgagcgtggacaagagccggtggcagcggggcgacaccttcatctgcgccgtgatgcacgaggccctgcacaaccactacacccaggagagcctgagccacagccccggc

SEQ ID NO: 191 - DNA, 1359 bp, synthetic construct
  source 1..1359: mol_type = other DNA
gaggtgcagctggtggagagcggcggcgacctggtgaagcccggcggcagcctgcggctgagctgcgtggccagcggcttcaccttcagcagctactggatgagctgggtgcggcaggcccccggcaagggcctgcagtggctgagcgacatctacgccaccggcagcacctactacgccaactgggccaccggccggttcaccggcagccgggacaacgccaagaacaccctgtacctgcagatgaacagcctgaccatcgaggacaccgccacctacttctgcgcccggcacagcagccggagcggcatcgacttcgacctgtggggccagggcaccctggtgaccgtgagcagcgccagcaccaccgcccccagcgtgttccccctggcccccagctgcggcagcaccagcggcagcaccgtggccctggcctgcctggtgagcggctacttccccgagcccgtgaccgtgagctggaacagcggcagcctgaccagcggcgtgcacaccttccccagcgtgctgcagagcagcggcctgtacagcctgagcagcatggtgaccgtgcccagcagccggtggcccagcgagaccttcacctgcaacgtggcccaccccgccagcaagaccaaggtggacaagcccgtgcccaagcgggagaacggccgggtgccccggccccccgactgccccaagtgccccgcccccgagatgctgggcggccccagcgtgttcatcttcccccccaagcccaaggacaccctgctgatcgcccggacccccgaggtgacctgcgtggtggtggacctggaccccgaggaccccgaggtgcagatcagctggttcgtggacggcaagcagatgcagaccgccaagacccagccccgggaggagcagttcaacggcacctaccgggtggtgagcgtgctgcccatcggccaccaggactggctgaagggcaagcagttcacctgcaaggtgaacaacaaggccctgcccagccccatcgagcggaccatcagcaaggcccggggccaggcccaccagcccagcgtgtacgtgctgccccccagccgggaggagctgagcaagaacaccgtgagcctgacctgcctgatcaaggacttcttcccccccgacatcgacgtggagtggcagagcaacggccagcaggagcccgagagcaagtaccggaccacccccccccagctggacgaggacggcagctacttcctgtacagcaagctgagcgtggacaagagccggtggcagcggggcgacaccttcatctgcgccgtgatgcacgaggccctgcacaaccactacacccaggagagcctgagccacagccccggc

SEQ ID NO: 192 - DNA, 1359 bp, synthetic construct; identical to SEQ ID NO: 191
  source 1..1359: mol_type = other DNA

SEQ ID NO: 193 - DNA, 648 bp, synthetic construct
  source 1..648: mol_type = other DNA
gagatcgtgatgacccagagccccgccagcctgagcctgagccagggcgagaaggtgaccatcacctgccaggccagccagagcatcagcatctacctggcctggtaccagcagaagcccggccagccccccaagctgctgatctaccgggccagcaccctggccagcggcgtgcccagccggttcagcggcagcggcagcggcaccgacttcagcttcaccatcaccagcctgcagcccgaggacgtggccacctactactgccagcagggctacgaggacagcaacgtggagagcctgttcggccagggcaccaaggtggagatcaagcggaacgacgcccagcccgccgtgtacctgttccagcccagccccgaccagctgcacaccggcagcgccagcgtggtgtgcctgctgaacagcttctaccccaaggacatcaacgtgaagtggaaggtggacggcgtgatccaggacaccggcatccaggagagcgtgaccgagcaggacaaggacagcacctacagcctgagcagcaccctgaccatgagcagcaccgagtacctgagccacgagctgtacagctgcgagatcacccacaagagcctgcccagcaccctgatcaagagcttccagcggagcgagtgc

SEQ ID NO: 194 - DNA, 648 bp, synthetic construct
  source 1..648: mol_type = other DNA
gagatcgtgatgacccagagccccgccagcctgagcctgagccagggcgagaaggtgaccatcacctgccaggccagccagagcatcagcatctacctggcctggtaccagcagaagcccggccagccccccaagctgctgatctaccgggccagcaccctggccagcggcgtgcccagccggttcagcggcagcggcagcggcaccgacttcagcttcaccatcagcagcctgcagcccgaggacgtggccacctactactgccagcagggctacgaggacagcaacgtggagagcctgttcggccagggcaccaaggtggagatcaagcggaacgacgcccagcccgccgtgtacctgttccagcccagccccgaccagctgcacaccggcagcgccagcgtggtgtgcctgctgaacagcttctaccccaaggacatcaacgtgaagtggaaggtggacggcgtgatccaggacaccggcatccaggagagcgtgaccgagcaggacaaggacagcacctacagcctgagcagcaccctgaccatgagcagcaccgagtacctgagccacgagctgtacagctgcgagatcacccacaagagcctgcccagcaccctgatcaagagcttccagcggagcgagtgc

SEQ ID NO: 195 - DNA, 717 bp, synthetic construct
  source 1..717: mol_type = other DNA
atggacacccgggcccccacccagctgctgggcctgctgctgctgtggctgcccggcgcccggtgcgagatcgtgatgacccagacccccgccagcctgagcctgagccaggagggcaaggtgaccatcacctgccaggccagcgagagcatcggcaacaccctggcctggtaccagcagaagcccggccagccccccaagctgctgatctacggcgccagcaccctggccagcggcgtgcccagccggttcagcggcagcggcagcggcaccgacttcaccttcaccatcaacagcctgcagcccgaggacgtggccacctactactgccagagctactggagcagcaccaccaccttccgggacaccttcggccagggcaccaaggtggagatcaagcggaacgacgcccagcccgccgtgtacctgttccagcccagccccgaccagctgcacaccggcagcgccagcgtggtgtgcctgctgaacagcttctaccccaaggacatcaacgtgaagtggaaggtggacggcgtgatccaggacaccggcatccaggagagcgtgaccgagcaggacaaggacagcacctacagcctgagcagcaccctgaccatgagcagcaccgagtacctgagccacgagctgtacagctgcgagatcacccacaagagcctgcccagcaccctgatcaagagcttccagcggagcgagtgc

SEQ ID NO: 196 - DNA, 717 bp, synthetic construct; identical to SEQ ID NO: 195
  source 1..717: mol_type = other DNA

SEQ ID NO: 197 - DNA, 648 bp, synthetic construct
  source 1..648: mol_type = other DNA
gagatcgtgatgacccagagccccgccagcctgagcctgagccaggaggagaaggtgaccatcacctgccaggccagccagagcatcggcaactacctggcctggtaccagcagaagcccggccaggcccccaagcagctgatctaccgggccagcaccctggccagcggcgtgcccagccggttcaagggcagcggcagcggcaccgagttcaccctgaccatcagcgacctggagtgcgccgacgtggccgtgtactactgccagcagggctacgacagcaacaacgtggacaacttcttcggccagggcaccaaggtggagatcaagcggaacgacgcccagcccgccgtgtacctgttccagcccagccccgaccagctgcacaccggcagcgccagcgtggtgtgcctgctgaacagcttctaccccaaggacatcaacgtgaagtggaaggtggacggcgtgatccaggacaccggcatccaggagagcgtgaccgagcaggacaaggacagcacctacagcctgagcagcaccctgaccatgagcagcaccgagtacctgagccacgagctgtacagctgcgagatcacccacaagagcctgcccagcaccctgatcaagagcttccagcggagcgagtgc

SEQ ID NO: 198 - DNA, 648 bp, synthetic construct
  source 1..648: mol_type = other DNA
gagatcgtgatgacccagagccccgccagcctgagcctgagccaggaggagaaggtgaccatcacctgccaggccagccagagcatcggcaactacctggcctggtaccagcagaagcccggccaggcccccaagcagctgatctaccgggccagcaccctggccagcggcgtgcccagccggttcagcggcagcggcagcggcaccgacttcaccctgaccatcagcgacctggagtgcgccgacgtggccgtgtactactgccagcagggctacgacagcaacaacgtggacaacttcttcggccagggcaccaaggtggagatcaagcggaacgacgcccagcccgccgtgtacctgttccagcccagccccgaccagctgcacaccggcagcgccagcgtggtgtgcctgctgaacagcttctaccccaaggacatcaacgtgaagtggaaggtggacggcgtgatccaggacaccggcatccaggagagcgtgaccgagcaggacaaggacagcacctacagcctgagcagcaccctgaccatgagcagcaccgagtacctgagccacgagctgtacagctgcgagatcacccacaagagcctgcccagcaccctgatcaagagcttccagcggagcgagtgc

SEQ ID NO: 199 - protein, 139 aa, synthetic construct
APTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQHHHHHH

SEQ ID NO: 200 - DNA, 420 bp, synthetic construct
  source 1..420: mol_type = other DNA
gcccccacccaccagctgccccccagcgacgtgcggaagatcatcctggagctgcagcccctgagccggggcctgctggaggactaccagaagaaggagaccggcgtgcccgagagcaaccggaccctgctgctgtgcctgaccagcgacagccagcccccccggctgaacagcagcgccatcctgccctacttccgggccatccggcccctgagcgacaagaacatcatcgacaagatcatcgagcagctggacaagctgaagttccagcacgagcccgagaccgagatcagcgtgcccgccgacaccttcgagtgcaagagcttcatcctgaccatcctgcagcagttcagcgcctgcctggagagcgtgttcaagagcctgaacagcggcccccagcaccaccaccaccaccactga